(19) United States
(12) Patent Application Publication
(10) Pub. No.: US 2025/0032478 A1
(43) Pub. Date: Jan. 30, 2025

(54) SOLID FORMS OF RESIQUIMOD AND FORMULATIONS THEREOF

(71) Applicant: SURGE Therapeutics, Inc., Cambridge, MA (US)

(72) Inventors: Paul Adam Konowicz, Beverly, MA (US); Michael Solomon Goldberg, Brookline, MA (US)

(21) Appl. No.: 18/716,267

(22) PCT Filed: Dec. 5, 2022
(86) PCT No.: PCT/US2022/051810

§ 371 Date: Jun. 4, 2024

Related U.S. Application Data
(60) Provisional application No. 63/286,361, filed on Dec. 6, 2021.

(51) Int. Cl. A61K 31/4745; A61K 9/06; A61K 47/10; A61K 47/36; A61P 35/00

(52) U.S. Cl. CPC A61K 31/4745; A61K 9/06; A61K 47/10; A61K 47/36; A61P 35/00

(57) ABSTRACT

The present disclosure provides solid forms of resiquimod useful, e.g., as an immunomodulatory payload component of certain biomaterials (e.g., hydrogels) and/or formulations for administration to subjects.

### RELATED APPLICATIONS

The present application claims priority to U.S. Application No. 63/286,361, filed Dec. 6, 2021, the entire contents of which are hereby incorporated by reference.

### BACKGROUND

Surgery is often the first-line of treatment for solid tumor cancers and is generally used in combination with systemic administration of anti-cancer therapy. However, surgery-induced immunosuppression has been implicated in the development of post-operative septic complications and tumor metastasis due to changes in a variety of metabolic and endocrine responses, ultimately resulting in the death of many patients (Hiller, J. G. et al. Nature Reviews Clinical Oncology, 2018, 15, 205-218).

Systemic administration of medication, nutrition, or other substances into the circulatory system affects the entire body. Systemic routes of administration include enteral (e.g., oral dosage resulting in absorption of the drug through the gastrointestinal tract) and parenteral (e.g., intravenous, intramuscular, and subcutaneous injections) administration. Administration of immunotherapeutics typically relies on these systemic administration routes, which can lead to unwanted side effects. In some instances, certain promising therapeutics are extremely difficult to develop due to associated toxicities and the limitations of current administration methods and systems.

Hydrogels are a particularly attractive type of biomaterial, and have been used in a wide range of applications, including tissue engineering and regenerative medicine, diagnostics, cellular immobilization, and/or drug delivery. However, existing hydrogels also have several limitations that restrict the practical use of hydrogel-based drug delivery therapies. For example, many hydrogels are usually formed outside of the body and then implanted, since bulk hydrogels have a defined dimensionality, which may make extrusion through a needle challenging. While some hydrogels may be formed in situ, there may be potential risks and challenges associated with certain crosslinking agents, e.g., UV radiation and/or crosslinking chemicals.

### SUMMARY

The present disclosure provides solid forms of resiquimod, as well as compositions thereof and methods of preparing the same. In some embodiments, provided solid forms are useful as an immunomodulatory payload component of certain biomaterials (e.g., hydrogels) and/or formulations for administration to, e.g., subjects who have undergone or are undergoing tumor resection. In some embodiments, provided solid forms display certain desirable characteristics, such as, e.g., certain solubility, stability, and/or hygroscopicity.

In some embodiments, the present disclosure provides methods of preparing formulations, the methods comprising providing a solid form of resiquimod. For example, in some embodiments, the present disclosure provides methods of preparing formulations suitable for intraoperative administration, the methods comprising providing a solid form of resiquimod.

In some embodiments, the present disclosure provides methods of using provided formulations of resiquimod.

### BRIEF DESCRIPTION OF THE DRAWINGS

FIG. 1 is an XRPD pattern of Resiquimod Form I.

FIG. 2 is a DSC thermogram of Resiquimod Form I.

FIG. 3 is a TGA thermogram of Resiquimod Form I.

FIG. 4 is an XRPD pattern of Resiquimod Form II.

FIG. 5 is a DSC thermogram of Resiquimod Form II.

FIG. 6 is a TGA thermogram of Resiquimod Form II.

FIG. 7 is an XRPD pattern of Resiquimod Form III (top spectrum) and an XRPD pattern of Resiquimod Form I, resulting from drying of Form III (bottom spectrum).

FIG. 8 is a DSC thermogram after drying a sample of Resiquimod Form III.

FIG. 9 is a TGA thermogram after drying a sample of Resiquimod Form III.

FIG. 10 is an XRPD pattern of Resiquimod Form IV.

FIG. 11 is a DSC thermogram of Resiquimod Form IV.

FIG. 12 is a TGA thermogram of Resiquimod Form IV.

FIG. 13 (top spectra) is an XRPD pattern of Resiquimod Form V. Upon grinding and/or drying, the sample converted into Resiquimod Form I (bottom three spectra).

FIG. 14 is a DSC thermogram of Resiquimod Form V.

FIG. 15 is a TGA thermogram of Resiquimod Form V.

FIG. 16 is an XRPD pattern of Resiquimod Form VI.

FIG. 17 is a DSC thermogram of Resiquimod Form VI.

FIG. 18 is a TGA thermogram of Resiquimod Form VI.

FIG. 19 is an XRPD pattern of Resiquimod Form VII.

FIG. 20 is a DSC thermogram of Resiquimod Form VII.

FIG. 21 is a TGA thermogram of Resiquimod Form VII.

FIG. 22A and FIG. 22B are heat maps depicting gelation properties of exemplary temperature-responsive polymer combination preparations comprising P407 at indicated concentrations in % (w/w) and hyaluronic acid (HA) having an average molecular weight of 1.5 MDa at indicated concentrations in % (w/w) in two different buffer systems. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted. FIG. 22A corresponds to 10 mM phosphate buffer saline (PBS) at pH 7.4.

FIG. 22B corresponds to 0.1 M bicarbonate buffer at pH 8.0.

FIG. 23A and FIG. 23B are heat maps depicting gelation properties of exemplary temperature-responsive polymer combination preparations comprising P407 at indicated concentrations in % (w/w) and hyaluronic acid (HA) having an average molecular weight of 730 kDa at indicated concentrations in % (w/w) in two different buffer systems. Polymer combination preparations were exposed to a temperature of 37° C. to observe any gel formation. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted. FIG. 23A corresponds to 10 mM PBS at pH 7.4. FIG. 23B corresponds to 0.1 M bicarbonate buffer at pH 8.0.

FIG. 24 is a heat map depicting gelation properties of exemplary temperature-responsive polymer combination preparations comprising P407 at indicated concentrations in % (w/w) and modified chitosan (e.g., carboxymethyl chitosan; CMCH) at indicated concentrations in % (w/w) in 10 mM PBS at pH 7.4. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted.

FIG. 25A and FIG. 25B are graphical representations showing storage modulus of exemplary temperature-responsive polymer combination preparations after exposure to a temperature of 37° C., as compared to control polymer compositions. FIG. 25A: Linear scale.

FIG. 25B: Logarithmic scale. Abbreviations: “18% P407”=18% (w/w) P407; “13.5% P407+0.65% 1.5 MDa HA (10 mM PBS)”=13.5% (w/w) P407+0.65% (w/w) 1.5 MDa HA in 10 mM PBS at pH 7.4; “13.5% P407+0.65% 1.5 MDa HA (0.1 M bicar)”=13.5% (w/w) P407+0.65% (w/w) 1.5 MDa HA in 0.1 M bicarbonate buffer at pH 8; “10% P407+1% 1.5 MDa HA (10 mM PBS)”=10% (w/w) P407+1% (w/w) 1.5 MDa HA in 10 mM PBS at pH 7.4; “13.5% P407+1.3% CMCH”=13.5% (w/w) P407+1.3% (w/w) CMCH in 10 mM PBS at pH 7.4; “12.5% Extralink”=Chemically-crosslinked hyaluronic acid with 12.5% Extralink thiol crosslinker; “1.5% Extralink”=Chemically-crosslinked hyaluronic acid with 1.5% Extralink thiol crosslinker; “0.5% Extralink”=Chemically-crosslinked hyaluronic acid with 0.5% Extralink thiol crosslinker.

FIG. 26A, FIG. 26B, FIG. 26C, and FIG. 26D are graphical representations showing homogeneity of exemplary temperature-responsive polymer combination preparations in a hydrogel state (when its precursor state was maintained at a temperature of 2-8° C. over a period of 1 month), with weekly measurements occurring at 37° C. (above CGT), as compared to control polymer compositions. Gel homogeneity was determined by measuring the storage modulus of hydrogels over a period of time. FIG. 26A: Control gel (18% w/w poloxamer 407); FIG. 26B: Temperature-responsive polymer combination preparation of 13.5% w/w poloxamer 407 and 0.65% w/w 1.5 MDa HA in 10 mM PBS at pH 7.4; FIG. 26C: Temperature-responsive polymer combination preparation of 10% w/w poloxamer 407 and 1% w/w 1.5 MDa HA in 10 mM PBS at pH 7.4; FIG. 26D: Temperature-responsive polymer combination preparation of 13.5% w/w poloxamer 407 and 0.65% w/w 1.5 MDa HA in 0.1 M bicarbonate buffer at pH 8.0.

FIG. 27A, FIG. 27B, FIG. 27C, FIG. 27D, and FIG. 27E are graphical representations showing in vivo survival data of tumor resection animals administered with exemplary temperature-responsive polymer combination preparations in a hydrogel state alone or incorporated with resiquimod, as compared to control chemically-crosslinked hyaluronic acid hydrogels alone or incorporated with resiquimod. The x-axis indicates time post-tumor inoculation. Tumor resection was performed at Day 10 post-tumor inoculation, and an exemplary composition was administered following the tumor resection. FIG. 27A: Control 12.5% (w/v) Extralink® Hyaluronic acid (HyStem™) hydrogel with or without resiquimod. FIG. 27B: Temperature-responsive polymer combination preparation of 10% w/w poloxamer 407 and 1% w/w 1.5 MDa HA in 10 mM PBS at pH 7.4, with or without resiquimod. FIG. 27C: Temperature-responsive polymer combination preparation of 13.5% w/w poloxamer 407 and 0.65% w/w 1.5 MDa HA in 10 mM PBS at pH 7.4, with or without resiquimod. FIG. 27D: Temperature-responsive polymer combination preparation of 13.5% w/w poloxamer 407 and 0.65% w/w 1.5 MDa HA in 0.1 M bicarbonate buffer at pH 8.0, with or without resiquimod. FIG. 27E: Temperature-responsive polymer combination preparation of 13.5% w/w poloxamer 407 and 1.3% w/w CMCH in 10 mM PBS at pH 7.4, with or without resiquimod.

FIG. 28A, FIG. 28B, FIG. 28C, and FIG. 28D are graphical representations showing in vivo survival data of tumor resection animals administered with exemplary temperature-responsive polymer combination preparations (e.g., a thermoresponsive liquid preparation comprising a combination of 730 kDa or 1.5 MDa Hyaluronic Acid (HA) at different concentrations and a poloxamer, e.g., P407), as a polymer combination alone or incorporated with resiquimod. The x-axis indicates time post-tumor inoculation. Tumor resection was performed at Day 10 post-tumor inoculation, and an exemplary composition was administered following the tumor resection. FIG. 28A: Temperature-responsive polymer combination preparation of 10% w/w poloxamer 407 and 2.25% w/w 730 kDa HA in 12.5 mM PBS at pH 8, with or without resiquimod. FIG. 28B: Temperature-responsive polymer combination preparation of 10% w/w poloxamer 407 and 2.25% w/w 730 kDa HA in 25 mM PBS at pH 8, with or without resiquimod. FIG. 28C: Temperature-responsive polymer combination preparation of 12.5% w/w poloxamer 407 and 1.625% 730 kDa HA in 25 mM PBS at pH 8, with or without resiquimod. FIG. 28D: Temperature-responsive polymer combination preparation of 8% w/w poloxamer 407 and 2.25% w/w 730 kDa HA in 25 mM buffered saline at pH 8, with or without resiquimod.

FIG. 29 is a graphical representation showing in vivo survival data of tumor resection animals administered with exemplary temperature-responsive polymer combination preparations (e.g., a thermoresponsive liquid preparation comprising a combination of 119 kDa Hyaluronic Acid (HA) with a poloxamer, e.g., P407), as a polymer combination alone or incorporated with resiquimod. Shown are results from temperature-responsive polymer combination preparation of 10% w/w poloxamer 407 and 4% w/w 119 kDa HA 25 mM buffered saline pH 7.4, with or without resiquimod. The x-axis indicates time post-tumor inoculation. Tumor resection was performed at Day 10 post-tumor inoculation, and an exemplary composition was administered following the tumor resection.

FIG. 30 is a graphical representation showing in vivo survival data of tumor resection animals administered with exemplary temperature-responsive polymer combination preparations (e.g., a thermoresponsive liquid preparation comprising a combination of 309 kDa Hyaluronic Acid (HA) with a poloxamer, e.g., P407), as a polymer combination alone or incorporated with resiquimod, or a poloxamer only control animal cohort. Shown are results from temperature-responsive polymer combination preparations of 10% w/w poloxamer 407 and 2% w/w 309 kDa HA 25 mM buffered saline at pH 7.4, with or without resiquimod, and a control preparation of 15% poloxamer 407 biomaterial without active agent. The x-axis indicates time post-tumor inoculation. Tumor resection was performed at Day 10 post-tumor inoculation, and an exemplary composition was administered following the tumor resection.

### DETAILED DESCRIPTION

### Definitions

It is noted that the concentrations of individual polymer components in polymer combination preparations described herein are each expressed in % (w/w) or wt %. As used herein, the concentration, % (w/w), of a polymer component in a polymer combination preparation is determined based on the mass or weight of the polymer component relative to the sum of (i) total mass or weight of all individual polymer components present in the polymer combination preparation and (ii) total mass or weight solvent used in the polymer combination preparation.

The term “about”, when used herein in reference to a value, refers to a value that is similar, in context to the referenced value. In general, those skilled in the art, familiar with the context, will appreciate the relevant degree of variance encompassed by “about” in that context. In some embodiments, the term “about” refers to +10% of a given value.

As used herein, the term “administer,” “administering,” or “administration” typically refers to the administration of a composition to a subject to achieve delivery of an agent or payload that is, or is included in, a composition to a target site or a site to be treated. Those of ordinary skill in the art will be aware of a variety of routes that may, in appropriate circumstances, be utilized for administration of different agents to a subject, for example a human. For example, while the terms “administer,” “administering,” or “administration” refer to implanting, absorbing, ingesting, injecting, inhaling, parenteral administration, or otherwise introducing a composition as described herein, in the context of administering a composition comprising a provided polymer combination preparation, administering may refer to, in some embodiments, implanting, or in some embodiments, injecting.

The term “biocompatible”, as used herein, refers to materials that do not cause significant harm to living tissue when placed in contact with such tissue, e.g., in vivo. Biocompatibility of a material can be gauged by the ability of such a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, and/or immunogenicity. In certain embodiments, materials are “biocompatible” if they themselves are not toxic to cells in an in vivo environment of its intended use. In certain embodiments, materials are “biocompatible” if their addition to cells in vitro results in less than or equal to 20% cell death and/or their administration in vivo does not induce significantly severe inflammation that is clinically undesirable for purposes described herein or other such adverse effects. As will be understood by those skilled in the art, such significantly severe inflammation is distinguishable from mild, transient inflammation, which typically accompanies surgery or introduction of foreign objects into a living organism. Furthermore, one of skill in the art will appreciate, reading the present disclosure, that in some embodiments, polymer combination preparations described herein and/or individual polymer components thereof are biocompatible if extent of immunomodulation (e.g., innate immunity agonism) over a defined period of time is clinically beneficial and/or desirable, e.g., to provide antitumor immunity.

As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significantly severe inflammation that is clinically undesirable for purposes described herein and/or other adverse effects in vivo. In some embodiments, biodegradable polymer materials break down into their component monomers. In some embodiments, biodegradable polymer materials may be biologically degraded, e.g., by enzymatic activity or cellular machinery, in some cases, for example, through exposure to a lysozyme (e.g., having relatively low pH), or by simple hydrolysis. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho)esters, polyesters, polyurethanes, poly(butyric acid), poly(valeric acid), poly(caprolactone), poly(hydroxyalkanoates), poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose variants and polyhydroxyalkanoates, for example, polyhydroxybutyrate blends and copolymers thereof. Those of ordinary skill in the art will appreciate or be able to determine when such polymers are biocompatible and/or biodegradable variants thereof (e.g., related to a parent polymer by substantially identical structure that differs only in substitution or addition of particular chemical groups as is known in the art).

The term “cancer” refers to a malignant neoplasm (Stedman's Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc.). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized.

The term “carbohydrate polymer” refers to a polymer that is or comprises one or more carbohydrates, e.g., having a carbohydrate backbone. For example, in some embodiments, a carbohydrate polymer refers to a polysaccharide or an oligosaccharide, or a polymer containing a plurality of monosaccharide units connected by covalent bonds. The monosaccharide units may all be identical, or, in some cases, there may be more than one type of monosaccharide unit present within the carbohydrate polymer. In certain embodiments, a carbohydrate polymer is naturally occurring. In certain embodiments, a carbohydrate polymer is synthetic (i.e., not naturally occurring). In some embodiments, a carbohydrate polymer may comprise a chemical modification. In some embodiments, a carbohydrate polymer is a linear polymer. In some embodiments, a carbohydrate polymer is a branched polymer.

As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those of ordinary skill in the art will also understand that when the term “comparable” is used in the context of comparison of two or more values, such values are comparable to one another such that the differences in values do not result in material differences in therapeutic outcomes, e.g., induction of anti-tumor immunity and/or incidence of tumor regrowth and/or metastasis. For example, in some embodiments, comparable release rates refer to values of such release rates within 15% over a period of 48 hours. In some embodiments, comparable release rates refer to values of such release rates within 20% over a period of 48 hours. In some embodiments, comparable release rates refer to values of such release rates within 15% over a period of 24 hours.

As used herein, the term “critical gelation temperature”, abbreviated as “CGT”, refers to a threshold temperature at or above which a precursor state of a polymer combination preparation (e.g., ones described herein) transitions to a polymer network state described herein (e.g., a hydrogel state). In some embodiments, a critical gelation temperature may correspond to a sol-gel transition temperature. In some embodiments, a critical gelation temperature may correspond to a lower critical solution temperature. See Taylor et al., “Thermoresponsive Gels” Gels (2017) 3:4, for general description of thermoresponsive gels, the contents of which are incorporated herein by reference for purposes described herein. As described in the present disclosure, certain embodiments of polymer combination preparations described herein are demonstrated to form a polymer network state when it is exposed to a temperature of about 35-40° C. One of ordinary skill in the art, reading the present disclosure, will understand that such polymer combination preparations do not necessarily have a CGT of about 35-40° C., but may rather have a CGT that is lower than 35-40° C. For example, in some embodiments, provided polymer combination preparations may have a CGT of about 20-28° C.

As used herein, the term “critical gelation weight ratio” refers to a threshold weight ratio of at least two or more polymer components in a provided polymer combination preparation, at or above which a precursor state of such a polymer combination preparation (e.g., ones described herein) transitions to a polymer network state described herein (e.g., a hydrogel state). In some embodiments, such a precursor-polymer network transition occurs when both a critical gelation temperature and a critical gelation weight ratio for a provided polymer combination preparation are achieved.

As used herein, the term “crosslink” refers to interaction and/or linkage between one entity and another entity to form a network. For example, in some embodiments, crosslinks present in polymer network may be or comprise intra-molecular crosslinks, inter-molecular crosslinks, or both. In some embodiments, crosslinks may comprise interactions and/or linkages between one polymer chain(s) and another polymer chain(s) to form a polymer network. In some embodiments, a crosslink may be achieved using one or more physical crosslinking approaches, including, e.g., one or more environmental triggers and/or physiochemical interactions. Examples of an environmental trigger include, but are not limited to pH, temperature, and/or ionic strength. Non-limiting examples of physiochemical interactions include hydrophobic interactions, charge interactions, hydrogen bonding interactions, stereocomplexation, and/or supramolecular chemistry. In some embodiments, a crosslink may be achieved using one or more covalent crosslinking approaches (e.g., where the linkage between two entities is or comprises a covalent bond) based on chemistry reactions, e.g., in some embodiments which may include reaction of an aldehyde and an amine to form a Schiff base, reaction of an aldehyde and hydrazide to form a hydrazine, and/or Michael reaction of an acrylate and either a primary amine or a thiol to form a secondary amine or a sulfide. Examples of such covalent crosslinking approaches include, but are not limited to small-molecule crosslinking and polymer-polymer crosslinking. Various methods for physical and covalent crosslinking of polymer chains are known in the art, for example, as described in Hoare and Kohane, “Hydrogels in drug delivery: Progress and challenges” Polymer (2008) 49:1993-2007, the entire content of which is incorporated herein by reference for the purposes disclosed herein.

As used interchangeably herein, the term “crosslinker” or “crosslinking agent” refers to an agent that links one entity (e.g., one polymer chain) to another entity (e.g., another polymer chain). In some embodiments, linkage (i.e., the “crosslink”) between two entities is or comprises a covalent bond. In some embodiments, linkage between two entities is or comprises an ionic bond or interaction. In some embodiments, a crosslinker is a chemical crosslinker, which, e.g., in some embodiments may be or comprise a small molecule (e.g., dialdehydes or genipin) for inducing formation of a covalent bond between an aldehyde and an amino group. In some embodiments, a crosslinker comprises a photo-sensitive functional group. In some embodiments, a crosslinker comprises a pH-sensitive functional group. In some embodiments, a crosslinker comprises a thermal-sensitive functional group.

The term “hydrogel” has its art-understood meaning and refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

As used herein, the term “poloxamer” refers to a polymer preparation of or comprising one or more poloxamers. In some embodiments, poloxamers in a polymer preparation may be unconjugated or unmodified, for example, which are typically triblock copolymers comprising a hydrophobic chain of polyoxypropylene (polypropylene glycol, PPG) flanked by two hydrophilic chains of polyoxyethylene (polyethylene glycol, PEG). In some embodiments, a polymer preparation of or comprising one or more poloxamer may be unfiltered (e.g., such a polymer preparation may contain impurities and/or relatively low molecular weight polymeric molecules, as compared to a comparable polymer preparation that is filtered or fractionated or otherwise purified). Examples of poloxamers include, but are not limited to, Poloxamer 124 (P124, also known as Pluronic L44 NF), Poloxamer 188 (P188, also known as Pluronic F68NF), Poloxamer 237 (P237, also known as Pluronic F 87 NF), Poloxamer 338 (P338, also known as Pluronic F108 NF), Poloxamer 407 (P407, also known as Pluronic F127 NF), and combinations thereof.

The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer (e.g., in a copolymer). In certain embodiments, a polymer is naturally occurring. In certain embodiments, a polymer is synthetic (i.e., not naturally occurring). In some embodiments, a polymer is a linear polymer. In some embodiments, a polymer is a branched polymer. In some embodiments, a polymer for use in accordance with the present disclosure is not a polypeptide. In some embodiments, a polymer for use in accordance with the present disclosure is not a nucleic acid.

As used herein, the term “polymer combination preparation” refers to a polymeric biomaterial comprising at least two distinct polymer components. For example, in many embodiments, a polymer combination preparation described herein is a polymeric biomaterial comprising a first polymer component and a second first polymer component, wherein the first polymer component is or comprises at least one poloxamer, and the second polymer component is or comprises a polymer that is not poloxamer. In some embodiments, a polymer combination preparation described herein is a polymeric biomaterial in a precursor state, which may be, e.g., useful for administration to a subject. In some embodiments, a polymer combination preparation described herein is a polymeric biomaterial in a polymer network state.

A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower) prior to receiving a composition described herein. In some embodiments, a tumor resection subject is a subject who has undergone tumor resection in less than 48 hours prior to receiving a composition described herein. In some embodiments, a tumor resection subject is a subject who has undergone tumor resection in less than 24 hours prior to receiving a composition described herein. In some embodiments, a tumor resection subject is a subject who has undergone tumor resection in less than 12 hours prior to receiving a composition described herein.

As used interchangeably herein, the term “sustained” or “extended” typically refers to prolonging an effect and/or a process over a desirable period of time. For example, in the context of sustained immunomodulation (e.g., in the presence of a composition or preparation as described and/or utilized herein), such an immunomodulatory effect may be observed for a longer period of time after administration of a particular immunomodulatory payload in the context of a composition comprising a biomaterial preparation and otherwise as described herein, as compared to that which is observed with administration of the same payload absent such a biomaterial preparation. In the context of sustained release of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) from compositions and/or preparations described herein over a period of time, such release may occur on a timescale within a range of from about 30 minutes to several weeks or more. In some embodiments, the extent of sustained release or extended release can be characterized in vitro or in vivo. For example, in some embodiments, release kinetics can be tested in vitro by placing a preparation and/or composition described herein in an aqueous buffered solution (e.g., PBS at pH 7.4). In some embodiments, when a preparation and/or composition described herein is placed in an aqueous buffered solution (e.g., PBS at pH 7.4), less than 100% or lower (including, e.g., less than or equal to 90%, less than or equal to 80%, less than or equal to 70%, less than or equal to 50% or lower) of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) is released within 3 hours from a biomaterial. In some embodiments, release kinetics can be tested in vivo, for example, by implanting a composition at a target site (e.g., mammary fat pad) of an animal subject (e.g., a mouse subject). In some embodiments, when a composition is implanted at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) is released in vivo 8 hours after the implantation.

As used herein, the term “temperature-responsive”, in the context of a temperature-responsive polymer or biomaterial (e.g., polymeric biomaterial), refers to a polymer or biomaterial (e.g., polymeric biomaterial) that exhibits an instantaneous or discontinuous change in one or more of its properties at a critical temperature (e.g., a critical gelation temperature). For example, in some embodiments, one or more of such properties is or comprise a polymer's or biomaterial's solubility in a particular solvent. By way of example only, in some embodiments, a temperature-responsive polymer or biomaterial (e.g., polymeric biomaterial) is characterized in that it is a homogenous polymer solution or colloid that is stable below a critical temperature (e.g., a critical gelation temperature) and instantaneously forms a polymer network (e.g., a hydrogel) when the critical temperature (e.g., critical gelation temperature) has been reached or exceeded. In some embodiments, a temperature-responsive polymer or biomaterial (e.g., polymeric biomaterial) may be temperature-reversible, e.g., in some embodiments where a polymer solution may instantaneously form a polymer network at a temperature of or above a critical gelation temperature, and such a resulting polymer network may instantaneously revert to a homogenous polymer solution when the temperature is reduced to below the critical gelation temperature.

The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., cancer or tumor. In some embodiments, treatment may be administered after one or more signs or symptoms have developed or have been observed. Treatment may also be continued after symptoms have resolved, for example, to delay or prevent recurrence and/or spread.

As used herein, the term “tumor resection subject” refers to a subject who is undergoing or has recently undergone a tumor resection procedure. In some embodiments, a tumor resection subject is a subject who has at least 70% or more (including, at least 80%, at least 90%, at least 95%, at least 98%, at least 99%, or higher (including 100%)) of gross tumor mass removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection subject may be determined to have a negative resection margin (i.e., no cancer cells seen microscopically at the resection margin, e.g., based on histological assessment of tissues surrounding the tumor resection site). In some embodiments, a tumor resection subject may be determined to have a positive resection margin (i.e., cancer cells are seen microscopically at the resection margin, e.g., based on histological assessment of tissues surrounding the tumor resection site). In some embodiments, a tumor resection subject may have micrometastases and/or dormant disseminated cancer cells that can be driven to progress/proliferate by the physiologic response to surgery. In some embodiments, a tumor resection subject receives a composition (e.g., as described and/or utilized herein) immediately after the tumor resection procedure is performed (e.g., intraoperative administration). In some embodiments, a tumor resection subject receives a composition (e.g., as described and/or utilized herein) postoperatively within 24 hours or less, including, e.g., within 18 hours, within 12 hours, within 6 hours, within 3 hours, within 2 hours, within 1 hour, within 30 mins, or less.

The term “tumor site” may, in some embodiments, be a site in which at least a portion of a tumor is present or was present prior to resection. In some embodiments, a tumor site may still have the entirety of the tumor present. While in some embodiments, a tumor site may have part or all of the tumor removed, e.g., through tumor resection.

### Resiquimod

Resiquimod (i.e., R-848) is an immune response modifier having the following structure:

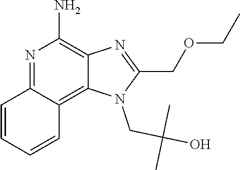 [SMILES: CCOCc1nc2c(N)nc3ccccc3c2n1CC(C)(C)O]

Resiquimod is a toll-like receptor 7 (TLR7) and toll-like receptor 8 (TLR8) agonist and has been shown to display antiviral and antitumor activity.

In some embodiments, resiquimod has been shown to be remarkably useful as an immunomodulatory payload component of certain biomaterials and/or formulations for administration to, e.g., subjects who have undergone or are undergoing tumor resection. See, for example, WO 2018/045058 or WO 2019/183216, each of which is hereby incorporated by reference in its entirety.

Without wishing to be bound by theory, the present disclosure provides the insight that it would be desirable to provide a form (e.g., a solid form) of resiquimod that, as compared to amorphous resiquimod and/or salt forms of resiquimod, imparts characteristics such as improved solubility, hygroscopicity, stability, and ease of formulation (e.g., particularly for use in formulations described herein). Accordingly, the present disclosure provides several solid forms of resiquimod, as well as methods of preparation and uses thereof.

### Solid Forms of Resiquimod

In some embodiments, the present disclosure provides a solid form of resiquimod. Resiquimod can exist in an amorphous solid form or in a crystalline solid form or in a mixture thereof. Crystalline solid forms can exist in one or more unique forms, which can be solvates, heterosolvates, hydrates, or unsolvated forms, etc. All such forms are contemplated by the present disclosure.

In some embodiments, the present disclosure provides one or more polymorphic solid forms of resiquimod. As used herein, the term “polymorph” refers to an ability of a compound to exist in one or more different crystal structures. For example, polymorphs may vary in pharmaceutically relevant physical properties, e.g., solubility, stability, and/or hygroscopicity.

In some embodiments, the present disclosure provides an unsolvated polymorphic form of resiquimod.

In some embodiments, the present disclosure provides resiquimod as a solvate or heterosolvate. As used herein, the term “solvate” refers to a solid form with a stoichiometric or non-stoichiometric amount of one or more solvents incorporated into the crystal structure. For example, a solvated or heterosolvated polymorph can independently comprise 0.05, 0.1, 0.2, 0.5, 1.0, 1.5, or 2.0, etc. equivalents of one or more solvents incorporated into the crystal lattice.

In some embodiments, the present disclosure provides resiquimod as a hydrate. As used herein, the term “hydrate” refers to a solvate wherein the solvent incorporated into the crystal structure is water.

As used herein, the term “about” when used in reference to a degree 2-theta value refers to the stated value±0.2 degrees 2-theta. In some embodiments, “about” refers to the stated value±0.1 degrees 2-theta.

### Resiquimod Form I

In some embodiments, a crystalline solid form of resiquimod is Resiquimod Form I. In some embodiments, Resiquimod Form I is unsolvated.

In some embodiments, Resiquimod Form I is characterized by one or more peaks in its XRPD pattern selected from those at about 8.72, about 12.24, about 16.29, about 17.56, about 19.51, about 21.31, and about 29.15 degrees 2-theta. In some embodiments, Resiquimod Form I is characterized by two or more peaks in its XRPD pattern selected from those at about 8.72, about 12.24, about 16.29, about 17.56, about 19.51, about 21.31, and about 29.15 degrees 2-theta. In some embodiments, Resiquimod Form I is characterized by three or more peaks in its XRPD pattern selected from those at about 8.72, about 12.24, about 16.29, about 17.56, about 19.51, about 21.31, and about 29.15 degrees 2-theta.

In some embodiments, Resiquimod Form I is characterized by peaks in its XRPD pattern at about 8.72, about 12.24, about 16.29, about 17.56, about 19.51, about 21.31, and about 29.15 degrees 2-theta. In some embodiments, Resiquimod Form I is characterized by an XRPD pattern comprising substantially all of the peaks selected from:
° 2θ±0.2
8.72
9.93
12.24
13.79
16.29
16.96
17.56
19.51
19.97
21.31
21.62
22.00
° 20±0.2
23.22
23.85
29.15

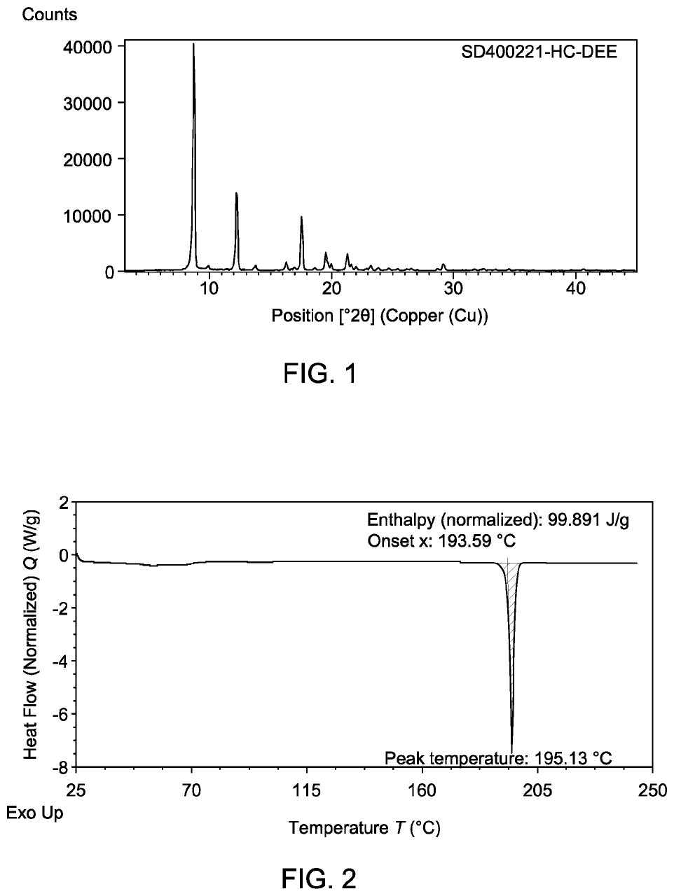

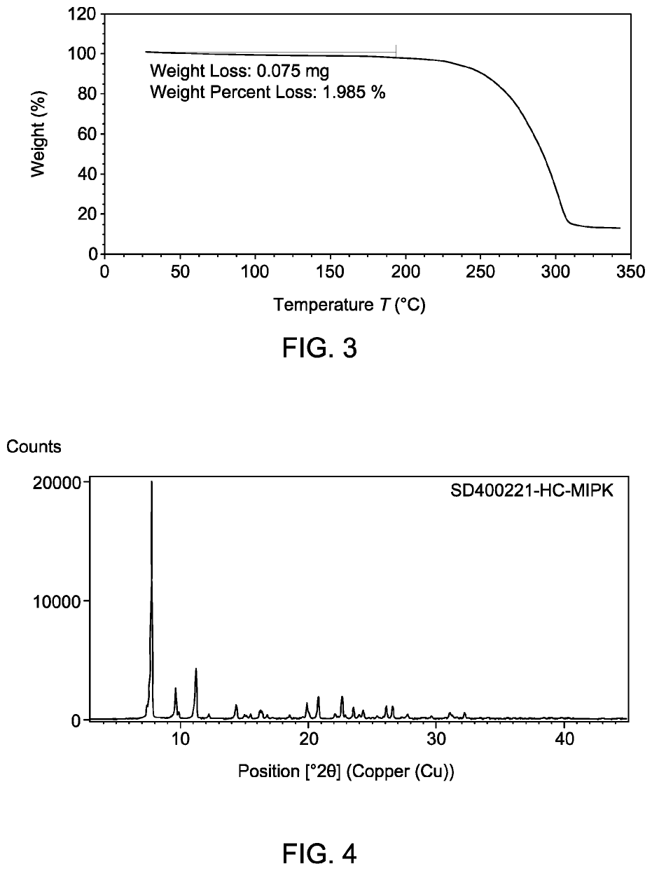

In some embodiments, Resiquimod Form I is characterized by one or more of the following:
(i) an XRPD pattern substantially similar to that depicted in FIG. 1;
(ii) a DSC thermogram substantially similar to that depicted in FIG. 2; and
(iii) a TGA thermogram substantially similar to that depicted in FIG. 3.

It will be appreciated, based on the data provided herein, that Form I displayed notably low hygroscopicity, making it particularly suitable for storing, handling, and formulating. Additionally, Form I has increased solubility in sodium phosphate buffered saline comprising 10 wt % poloxamer 407, indicating, e.g., that it is suitable for use in certain formulations, such as those described herein.

### Resiquimod Form II

In some embodiments, a crystalline solid form of resiquimod is Resiquimod Form II. In some embodiments, Resiquimod Form II is a methyl isopropyl ketone solvate.

In some embodiments, Resiquimod Form II is characterized by one or more peaks in its XRPD pattern selected from those at about 7.75, about 9.65, about 11.23, about 14.38, about 19.90, about 20.80, and about 22.65 degrees 2-theta. In some embodiments, Resiquimod Form II is characterized by two or more peaks in its XRPD pattern selected from those at about 7.75, about 9.65, about 11.23, about 14.38, about 19.90, about 20.80, and about 22.65 degrees 2-theta. In some embodiments, Resiquimod Form II is characterized by three or more peaks in its XRPD pattern selected from those at about 7.75, about 9.65, about 11.23, about 14.38, about 19.90, about 20.80, and about 22.65 degrees 2-theta.

In some embodiments, Resiquimod Form II is characterized by peaks in its XRPD pattern at about 7.75, about 9.65, about 11.23, about 14.38, about 19.90, about 20.80, and about 22.65 degrees 2-theta. In some embodiments, Resiquimod Form II is characterized by an XRPD pattern comprising substantially all of the peaks selected from:
° 2θ±0.2
7.39
7.75
9.65
9.89
11.23
12.25
14.38
15.00
15.50
16.23
16.38
16.80
18.55
19.90
20.80
22.11
22.65
22.90
23.54
23.99
24.29
25.38
26.10
26.61
27.78
29.63
31.04
32.21

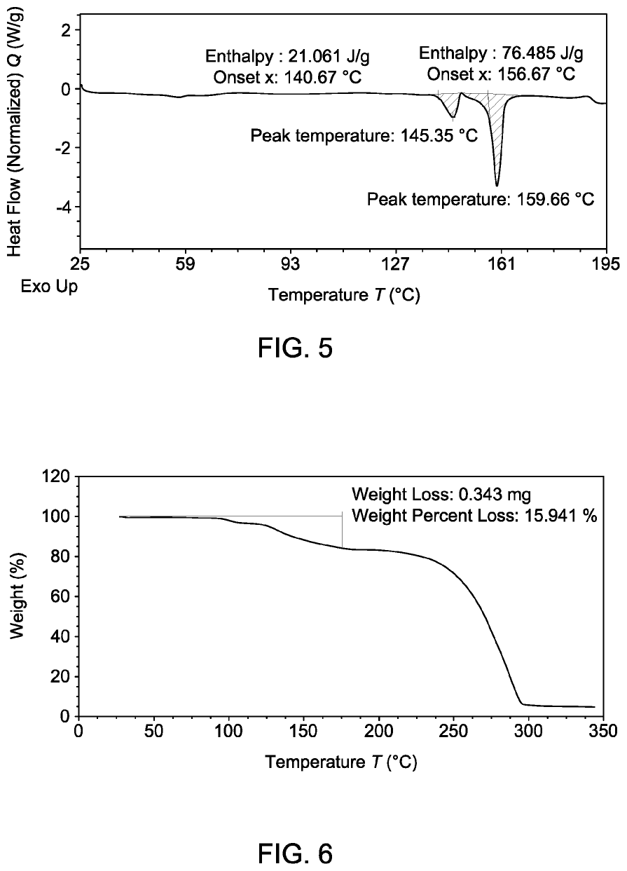

In some embodiments, Resiquimod Form II is characterized by one or more of the following:
(i) an XRPD pattern substantially similar to that depicted in FIG. 4;
(ii) a DSC thermogram substantially similar to that depicted in FIG. 5; and
(iii) a TGA thermogram substantially similar to that depicted in FIG. 6.

### Resiquimod Form III

In some embodiments, a crystalline solid form of resiquimod is Resiquimod Form III. In some embodiments, Resiquimod Form III is unsolvated.

In some embodiments, Resiquimod Form III is characterized by one or more peaks in its XRPD pattern selected from those at about 8.69, about 9.18, about 9.48, about 11.97, about 14.41, about 18.53, and about 19.70 degrees 2-theta. In some embodiments, Resiquimod Form III is characterized by two or more peaks in its XRPD pattern selected from those at about 8.69, about 9.18, about 9.48, about 11.97, about 14.41, about 18.53, and about 19.70 degrees 2-theta. In some embodiments, Resiquimod Form III is characterized by three or more peaks in its XRPD pattern selected from those at about 8.69, about 9.18, about 9.48, about 11.97, about 14.41, about 18.53, and about 19.70 degrees 2-theta.

In some embodiments, Resiquimod Form III is characterized by peaks in its XRPD pattern at about 8.69, about 9.18, about 9.48, about 11.97, about 14.41, about 18.53, and about 19.70 degrees 2-theta. In some embodiments, Resiquimod Form III is characterized by an XRPD pattern comprising substantially all of the peaks selected from:
° 2θ+0.2
8.69
9.18
9.48
10.61
11.97
12.26
14.41
15.47
16.04
17.54
17.95
18.53
19.26
19.70
20.57
21.09
22.10
22.43
23.13
23.76
24.13
27.20
28.00
28.42
29.14
33.06
34.06

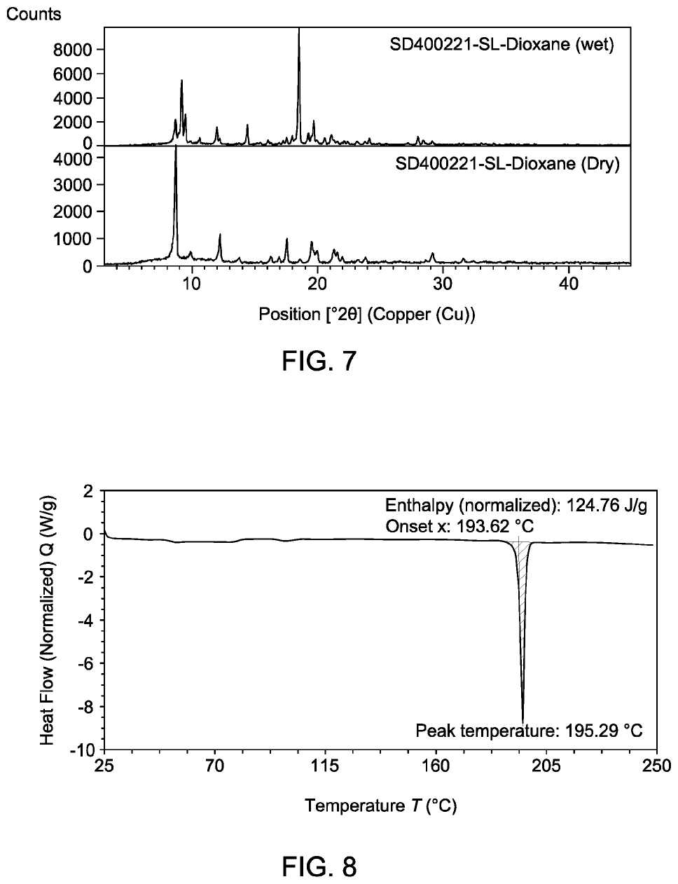

In some embodiments, Resiquimod Form III is characterized by an XRPD pattern substantially similar to that depicted in FIG. 7 (top).

### Resiquimod Form IV

In some embodiments, a crystalline solid form of resiquimod is Resiquimod Form IV. In some embodiments, Resiquimod Form IV is a solvate.

In some embodiments, Resiquimod Form IV is characterized by one or more peaks in its XRPD pattern selected from those at about 6.01, about 12.00, about 12.15, about 16.14, about 19.24, about 20.21, about 21.19, about 22.12, and about 24.50 degrees 2-theta. In some embodiments, Resiquimod Form IV is characterized by two or more peaks in its XRPD pattern selected from those at about 6.01, about 12.00, about 12.15, about 16.14, about 19.24, about 20.21, about 21.19, about 22.12, and about 24.50 degrees 2-theta. In some embodiments, Resiquimod Form IV is characterized by three or more peaks in its XRPD pattern selected from those at about 6.01, about 12.00, about 12.15, about 16.14, about 19.24, about 20.21, about 21.19, about 22.12, and about 24.50 degrees 2-theta.

In some embodiments, Resiquimod Form IV is characterized by peaks in its XRPD pattern at about 6.01, about 12.00, about 12.15, about 16.14, about 19.24, about 20.21, about 21.19, about 22.12, and about 24.50 degrees 2-theta. In some embodiments, Resiquimod Form IV is characterized by an XRPD pattern comprising substantially all of the peaks selected from:
° 2θ+0.2
6.01
10.30
10.51
12.00
12.15
14.79
16.14
17.16
17.86
19.24
20.21
20.80
21.19
21.70
22.12
22.87
23.42
° 2θ+0.2
23.75
24.50
25.01
26.02
26.83
28.23
29.53
30.19
35.02

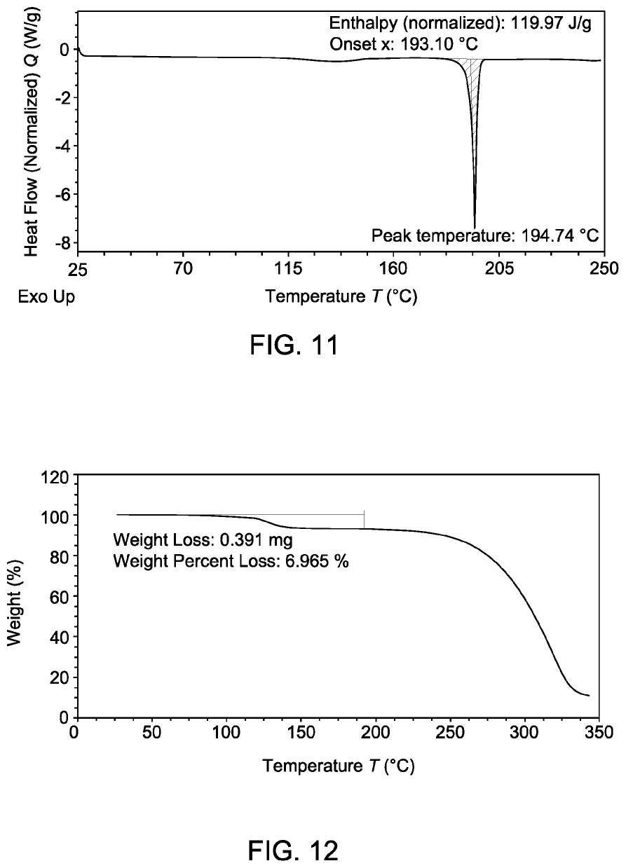

In some embodiments, Resiquimod Form IV is characterized by one or more of the following:
(i) an XRPD pattern substantially similar to that depicted in FIG. 10;
(ii) a DSC thermogram substantially similar to that depicted in FIG. 11; and
(iii) a TGA thermogram substantially similar to that depicted in FIG. 12.

### Resiquimod Form V

In some embodiments, a crystalline solid form of resiquimod is Resiquimod Form V. In some embodiments, Resiquimod Form V is unsolvated.

In some embodiments, Resiquimod Form V is characterized by one or more peaks in its XRPD pattern selected from those at about 8.13, about 10.20, about 10.44, about 16.29, and about 24.56 degrees 2-theta. In some embodiments, Resiquimod Form V is characterized by two or more peaks in its XRPD pattern selected from those at about 8.13, about 10.20, about 10.44, about 16.29, and about 24.56 degrees 2-theta. In some embodiments, Resiquimod Form V is characterized by three or more peaks in its XRPD pattern selected from those at about 8.13, about 10.20, about 10.44, about 16.29, and about 24.56 degrees 2-theta.

In some embodiments, Resiquimod Form V is characterized by peaks in its XRPD pattern at about 8.13, about 10.20, about 10.44, about 16.29, and about 24.56 degrees 2-theta. In some embodiments, Resiquimod Form V is characterized by an XRPD pattern comprising substantially all of the peaks selected from:
° 2θ+0.2
8.13
10.20
10.44
16.29
24.56
32.97
41.53

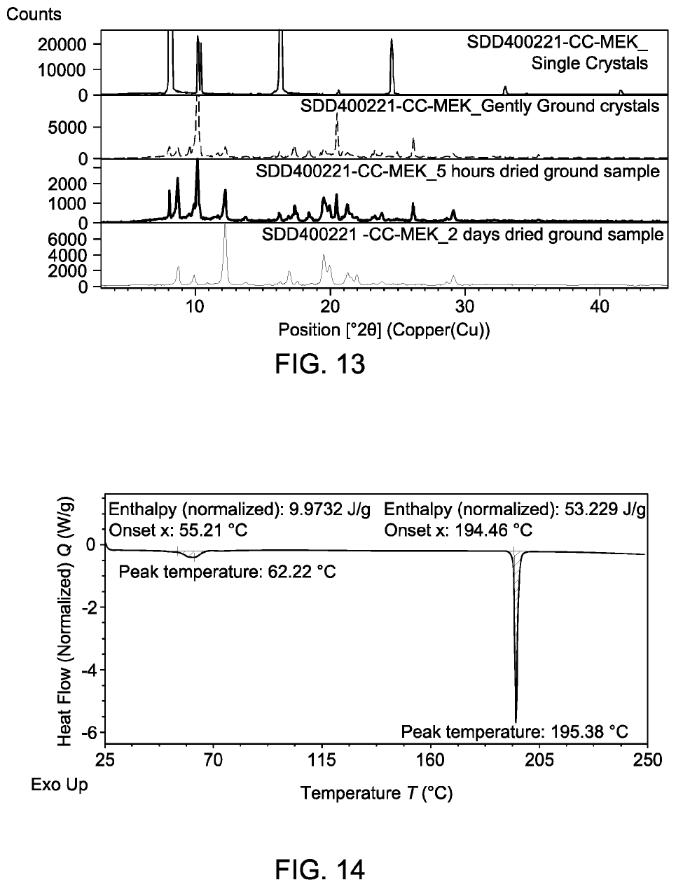

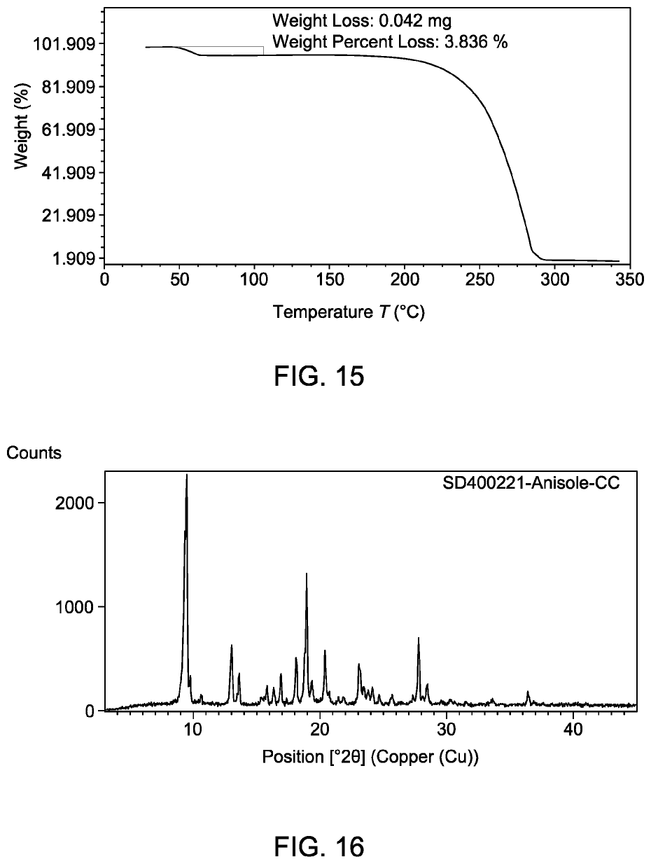

In some embodiments, Resiquimod Form V is characterized by one or more of the following:
(i) an XRPD pattern substantially similar to that depicted in FIG. 13 (top);
(ii) a DSC thermogram substantially similar to that depicted in FIG. 14; and
(iii) a TGA thermogram substantially similar to that depicted in FIG. 15.

### Resiquimod Form VI

In some embodiments, a crystalline solid form of resiquimod is Resiquimod Form VI. In some embodiments, Resiquimod Form VI is an anisole solvate.

In some embodiments, Resiquimod Form VI is characterized by one or more peaks in its XRPD pattern selected from those at about 9.40, about 13.02, about 18.13, about 18.93, about 20.38, about 23.16, and about 27.78 degrees 2-theta. In some embodiments, Resiquimod Form VI is characterized by two or more peaks in its XRPD pattern selected from those at about 9.40, about 13.02, about 18.13, about 18.93, about 20.38, about 23.16, and about 27.78 degrees 2-theta. In some embodiments, Resiquimod Form VI is characterized by three or more peaks in its XRPD pattern selected from those at about 9.40, about 13.02, about 18.13, about 18.93, about 20.38, about 23.16, and about 27.78 degrees 2-theta.

In some embodiments, Resiquimod Form VI is characterized by peaks in its XRPD pattern at about 9.40, about 13.02, about 18.13, about 18.93, about 20.38, about 23.16, and about 27.78 degrees 2-theta. In some embodiments, Resiquimod Form VI is characterized by an XRPD pattern comprising substantially all of the peaks selected from:
° 2θ+0.2
9.40
10.66
13.02
13.60
° 2θ+0.2
15.80
16.35
16.90
17.36
18.13
18.93
19.32
20.38
21.85
23.12
24.14
24.68
25.71
27.78
28.45
29.62
33.68
36.43

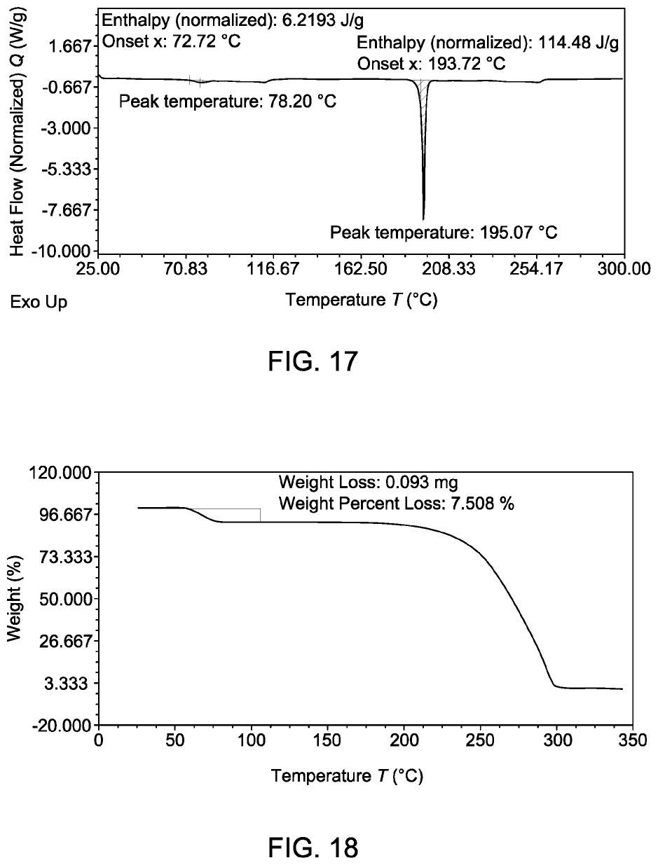

In some embodiments, Resiquimod Form VI is characterized by one or more of the following:
(i) an XRPD pattern substantially similar to that depicted in FIG. 16;
(ii) a DSC thermogram substantially similar to that depicted in FIG. 17; and
(iii) a TGA thermogram substantially similar to that depicted in FIG. 18.

### Resiquimod Form VII

In some embodiments, a crystalline solid form of resiquimod is Resiquimod Form VII.

In some embodiments, Resiquimod Form VII is characterized by one or more peaks in its XRPD pattern selected from those at about 6.25, about 9.92, about 10.96, about 16.51, about 18.99, about 23.75, and about 24.24 degrees 2-theta. In some embodiments, Resiquimod Form VII is characterized by two or more peaks in its XRPD pattern selected from those at about 6.25, about 9.92, about 10.96, about 16.51, about 18.99, about 23.75, and about 24.24 degrees 2-theta. In some embodiments, Resiquimod Form VII is characterized by three or more peaks in its XRPD pattern selected from those at about 6.25, about 9.92, about 10.96, about 16.51, about 18.99, about 23.75, and about 24.24 degrees 2-theta.

In some embodiments, Resiquimod Form VII is characterized by peaks in its XRPD pattern at about 6.25, about 9.92, about 10.96, about 16.51, about 18.99, about 23.75, and about 24.24 degrees 2-theta. In some embodiments, Resiquimod Form VII is characterized by an XRPD pattern comprising substantially all of the peaks selected from:
° 2θ+0.2
6.25
8.26
9.92
10.96
12.01
12.35
12.89
14.09
14.64
15.53
16.09
16.51
17.58
18.06
18.99
20.01
20.34
20.78
21.07
21.63
22.10
23.07
23.75
24.24
24.60
24.87
25.59
26.33
26.61
26.92
27.64
29.72
30.53
31.96
36.13
36.63
37.98

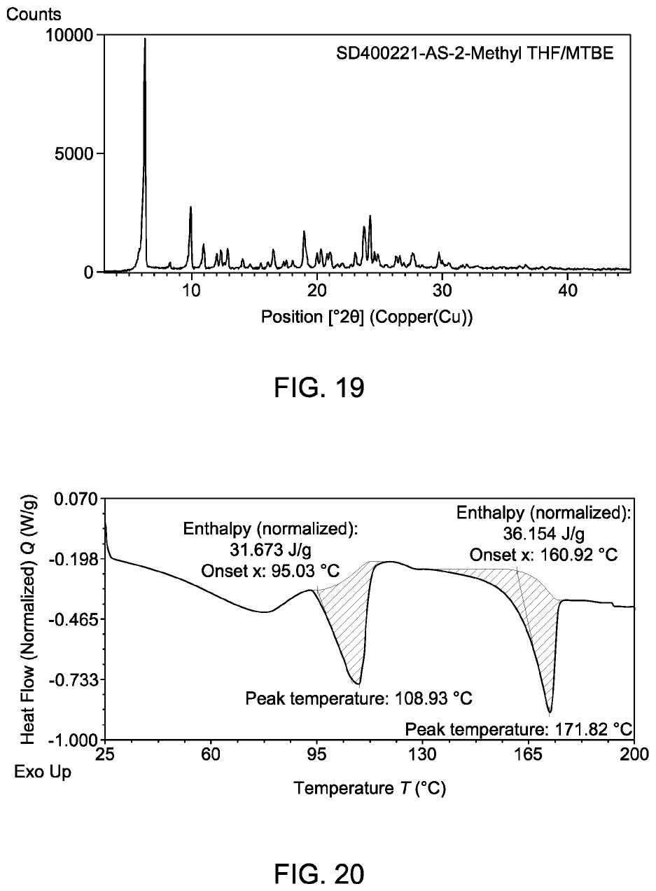

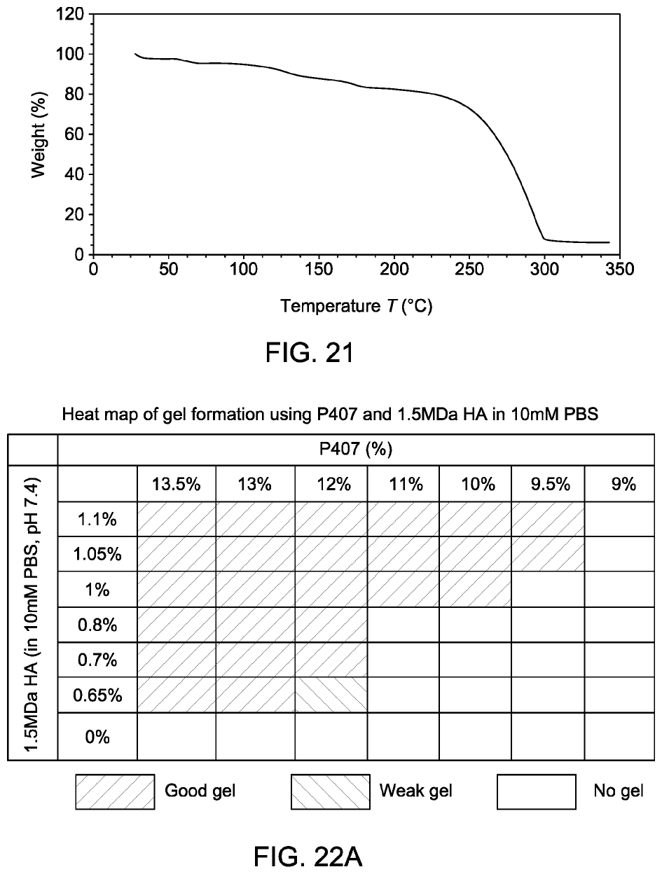

In some embodiments, Resiquimod Form VII is characterized by one or more of the following:
(i) an XRPD pattern substantially similar to that depicted in FIG. 19;
(ii) a DSC thermogram substantially similar to that depicted in FIG. 20; and
(iii) a TGA thermogram substantially similar to that depicted in FIG. 21 Methods of Preparing Provided Solid Forms

In some embodiments, the present disclosure provides methods of preparing provided solid forms of resiquimod (e.g., Resiquimod Form I, Form II, Form III, Form IV, Form V, Form VI, and Form VII).

In some embodiments, provided solid forms of resiquimod are prepared by dissolving resiquimod (e.g., crystalline and/or amorphous resiquimod) in a suitable solvent and then causing resiquimod to return to the solid phase. In some embodiments, solid forms of resiquimod are prepared by combining amorphous and/or crystalline resiquimod in a suitable solvent under suitable conditions and isolating a solid form of resiquimod.

In some embodiments, a suitable solvent is selected from 1-butanol, 1-propanol, 2,2-dimethoxypropane, 2-butanol, 2-methoxyethanol, 2-methyltetrahydrofuan, 2-propanol, acetone, acetonitrile, amyl alcohol, anisole, chloroform, cyclohexane, cyclopentyl methyl ether, dichloromethane, diethyl ether, dioxane, ethanol, ethyl acetate, heptane, hexane, isobutanol, isobutyl acetate, isopropyl acetate, m-xylene, methanol, methyl acetate, methyl ethyl ketone, methyl isobutyl ketone, methyl isopropyl ketone, methyl t-butyl ether, nitromethane, octane, pentane, petroleum ether, tetrahydrofuran, toluene, water, and xylene, or any combination thereof.

In some embodiments, a method of preparing a solid form of resiquimod comprises a step of heating a mixture comprising resiquimod and a suitable solvent (e.g., a suitable solvent described herein) to a suitable temperature. In some such embodiments, a suitable temperature is from about 40° C. to about 60° C.

In some embodiments, a method of preparing a solid form of resiquimod comprises cooling a mixture comprising resiquimod and a suitable solvent (e.g., a suitable solvent described herein) to a suitable temperature. In some such embodiments, a suitable temperature is from about 0° C. to about 10° C.

In some embodiments, a method of preparing a solid form of resiquimod comprises iterative heating and cooling cycles, wherein a mixture comprising resiquimod and a suitable solvent (e.g., a suitable solvent described herein) is heated to a suitable temperature for a period of time and then cooled to a suitable temperature for a period of time. In some embodiments, the heating and cooling cycles are repeated, e.g., for 2, 3, 4, 5, or 6 cycles.

In some embodiments, a method of preparing a solid form of resiquimod comprises slurrying resiquimod in a suitable solvent (e.g., a suitable solvent described herein) at a suitable temperature (e.g., from about 40° C. to about 60° C.).

In some embodiments, a solid form of resiquimod precipitates from a mixture (e.g., from a solution, suspension, or slurry). In some embodiments, a solid form of resiquimod crystallizes from solution. In some embodiments, a solid form of resiquimod crystallizes from solution following seeding of the solution (e.g., adding crystals of resiquimod to the solution). In some embodiments, a solid form of resiquimod precipitates or crystallizes from a mixture after removal of part or all of a solvent via methods such as evaporation, distillation, or filtration. In some embodiments, a solid form of resiquimod precipitates or crystallizes from a mixture after addition of a suitable anti-solvent (e.g., water, heptane, hexane, or methyl t-butyl ether). For example, in some embodiments, Resiquimod Form I is prepared by dissolving resiquimod in a suitable solvent (e.g., dichloromethane) and adding a suitable anti-solvent (e.g., heptane), thereby causing Resiquimod Form I to form. In some embodiments, a solid form of resiquimod precipitates or crystallizes from a mixture upon cooling to a suitable temperature (e.g., about −20° C., about 0° C., or about 5° C.).

In some embodiments, a method of preparing a solid form of resiquimod comprises a step of isolating a solid form of resiquimod. It will be appreciated that a solid form of resiquimod may be isolated by any suitable means. In some embodiments, a solid form of resiquimod (e.g., precipitated or crystallized resiquimod) is separated from a supernatant by filtration. In some embodiments, a solid form of resiquimod (e.g., precipitated or crystallized resiquimod) is separated from a supernatant by decanting the supernatant.

In some embodiments, a solid form of resiquimod is dried (e.g., in air or under reduced pressure and optionally at elevated temperature).

In some embodiments, a solid form of resiquimod is prepared by converting one solid form of resiquimod to another solid form of resiquimod.

### Compositions of Provided Solid Forms

The present disclosure also provides compositions comprising one or more solid forms of resiquimod. In some embodiments, provided compositions comprise crystalline resiquimod (e.g., Resiquimod Form I, Resiquimod Form II, Resiquimod Form III, Resiquimod Form IV, Resiquimod Form V, Resiquimod Form VI, or Resiquimod Form VII). In some embodiments, provided compositions comprise amorphous resiquimod.

In some embodiments, a provided composition comprises crystalline resiquimod and amorphous resiquimod. In some embodiments, a composition comprising crystalline resiquimod is substantially free of amorphous resiquimod. As used herein, the term “substantially free of amorphous resiquimod” means that the composition contains no significant amount of an amorphous solid form. In some embodiments, a composition comprises at least about 90% by weight of crystalline resiquimod. In some embodiments, a composition comprises at least about 95% by weight of crystalline resiquimod. In some embodiments, a composition comprises at least about 97%, about 98%, or about 99% by weight of crystalline resiquimod. In some embodiments, a composition comprises no more than about 10% by weight of amorphous resiquimod. In some embodiments, a composition comprises no more than about 5% by weight of amorphous resiquimod. In some embodiments, a composition comprises no more than about 3%, about 2%, or about 1% by weight of amorphous resiquimod.

In some embodiments, provided compositions comprising crystalline resiquimod are substantially free of impurities. As used herein, the term “substantially free of impurities” means that the composition contains no significant amount of extraneous matter. Such extraneous matter may include starting materials, residual solvents, or any other impurities that may result from the preparation and/or isolation of crystalline resiquimod. In some embodiments, a provided composition comprises no more than about 10% by weight impurities. In some embodiments, a provided composition comprises no more than about 5% by weight impurities. In some embodiments, a provided composition comprises no more than about 3%, about 2%, or about 1% by weight impurities.

In some embodiments, a composition comprises a mixture of crystalline solid forms of resiquimod (e.g., a mixture comprising two or more of Resiquimod Form I, Resiquimod Form II, Resiquimod Form III, Resiquimod Form IV, Resiquimod Form V, Resiquimod Form VI, and Resiquimod Form VII). In some embodiments, a composition comprises a mixture of Resiquimod Form I and one or more other crystalline solid forms of resiquimod (e.g., one or more of Resiquimod Form II, Resiquimod Form III, Resiquimod Form IV, Resiquimod Form V, Resiquimod Form VI, and Resiquimod Form VII).

### Biomaterial Compositions

The present disclosure also provides certain biomaterial formulations and/or polymer combination compositions, comprising resiquimod. In some embodiments, provided solid forms are useful in the preparation of such compositions.

Various systems involving a combination of a biomaterial and an immunomodulatory payload (see, for example WO 2018/045058 or WO 2019/183216) are reported to be remarkably useful, among other things, when administered to subjects who have undergone or are undergoing tumor resection. Attributes of these systems addressed the source of one or more problems associated with certain prior technologies including, for example, certain conventional approaches to cancer treatment. For example, this system could reduce and/or avoid certain adverse events (e.g., skin rashes, hepatitis, diarrhea, colitis, hypophysitis, thyroiditis, and adrenal insufficiency) that can be associated with systemic administration of immunotherapeutic agents. Among other things, this system could reduce or eliminate exposure of non-tumor-specific immune cells to systemically-administered immunotherapeutic drug(s) and/or to high doses of such drug(s) that are often required in order for systemic administration to achieve sufficient concentration in the tumor to induce a desired response; among other things, the system could provide local immunomodulation (e.g., local agonism of innate immunity) following tumor resection, which, among other things, can improve efficacy by concentrating the immunomodulatory effect where it is needed. Additionally or alternatively, such systems that provide local immunomodulation (e.g., agonism of innate immunity) following resection can, among other things, break local immune tolerance toward cancer and allow for development of systemic antitumor immunity, which can, for example, in some embodiments, lead to eradiation of disseminated disease.

The present disclosure provides certain such biomaterial formulations that may be particularly useful and/or may provide particular beneficial effects, e.g., as described herein. In some embodiments, provided solid forms are useful for preparing such biomaterial formulations.

In some embodiments, the present disclosure appreciates the source of a problem with certain prior technologies including, for example, with certain crosslinked biopolymer materials. Among other things, the present disclosure appreciates that certain crosslinking technologies may produce toxic by-products and/or may adversely affect stability and/or efficacy of agent(s) (e.g., therapeutic agents, e.g., resiquimod) when combined with biopolymer materials before or during crosslinking.

Alternatively or additionally, the present disclosure appreciates the source of a problem with technologies that involve pre-forming (e.g., by cross-linking) a biopolymer material prior to introducing it into a subject. For example, the present disclosure appreciates that such pre-forming generates a material with a defined size and/or structure, which may restrict options for administration. The present disclosure provides technologies, including particular biomaterial preparations, that permit administration by a variety of routes and/or approaches, including by methods, such as injection and/or laparoscopic administration, that may be less invasive than implantation. In some such embodiments, preparations with improved administration characteristics may be administered in a liquid state; in some embodiments they may be administered in a pre-formed gel state characterized by flexible space-filling properties. In some such embodiments, provided preparations are comprised of a relevant material in particulate form (e.g., so that the preparations comprise a plurality of particles, e.g., characterized by a size distribution and/or other parameters as described herein).

Among other things, in some embodiments, the present disclosure provides temperature-responsive biomaterial preparations that, for example, are able to transition from an injectable state to another state with material properties that provide beneficial effects, e.g., as described herein, without introduction of a cytotoxic crosslinking agent, e.g., UV radiation and/or small-molecule crosslinkers. Some such embodiments thus provide valuable technologies for in situ formation of gelled materials, which technologies have various benefits relative to alternative technologies, and provide a solution to certain problems with such alternative technologies as identified herein. For example, the present disclosure appreciates the source of a problem with various alternative technologies for in situ gelation, as many such technologies require treatments (e.g., exposure to UV radiation and/or to a small-molecule crosslinker, that may have toxic or otherwise damaging effects for the recipient and/or for an agent (e.g., resiquimod) that may be included in or with the material.

In some embodiments, provided temperature-responsive biomaterial preparations (e.g., ones described herein) may demonstrate one or more immunomodulatory attributes. For example, in some embodiments, provided temperature-responsive biomaterial preparations may promote innate immunity upon administration to a target site in a subject in need thereof (e.g., tumor resection subjects).

In some embodiments, the present disclosure appreciates, among other things, that certain conventional preparations that are or comprise a poloxamer and that are used to form a hydrogel typically utilize a poloxamer (e.g., Poloxamer 407 (P407)) at a minimum concentration of 16-20% (w/w). The present disclosure appreciates the source of a problem with such conventional preparations, including that they may have certain disadvantages for administration to subjects, including, e.g., high solution viscosity that makes it less ideal for injection, and/or tissue irritation due to high concentrations of poloxamers. Moreover, the present disclosure demonstrates that it is possible to develop useful preparations with materially lower concentration(s) of such poloxamers.

For example, in some embodiments, the present disclosure appreciates that certain poloxamers, e.g., Poloxamer 407 (P407), which have been typically used at a minimum concentration of 16-20% (w/w) to form a hydrogel, can form a useful temperature-responsive biomaterial at a concentration lower than 16% (w/w), including, e.g., lower than 14% (w/w), lower than 12% (w/w), lower than 11% (w/w), lower than 10.5% (w/w), lower than 10% (w/w), lower than 8% (w/w), lower than 6% (w/w), or lower, when combined with one or more biocompatible polymers. In some embodiments, such biocompatible polymers may be or comprise a polymer that is not temperature-responsive, e.g., in some embodiments which may be or comprise hyaluronic acid and/or chitosan or modified chitosan.

One aspect provided herein relates to a preparation or composition comprising a polymer combination preparation comprising at least first and second polymer components, the first polymer component is or comprises a poloxamer and the second polymer component is not a poloxamer, wherein the first polymer component is present in the polymer combination preparation at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4% (w/w), or lower). In some embodiments, a first polymer component is present in a polymer combination preparation at a concentration of 4% (w/w) to 11% (w/w), or 4% (w/w) to 10.5% (w/w), or 4% (w/w) to 10% (w/w). In some embodiments, a first polymer component is present in a polymer combination preparation at a concentration of 5% (w/w) to 11% (w/w), or 5% (w/w) to 10.5% (w/w), or 5% (w/w) to 10% (w/w). In some embodiments, a first polymer component is present in a polymer combination preparation at a concentration of 6% (w/w) to 11% (w/w), or 6% (w/w) to 10.5% (w/w), or 6% (w/w) to 10% (w/w). In some embodiments, such a polymer combination preparation is characterized in that it transitions from a precursor state to a polymer network state in response to a gelation trigger. Such a gelation trigger is or comprises one or more of the following: (a) temperature at or above critical gelation temperature (CGT) for the polymer combination preparation, (b) critical gelation weight ratio of the first polymer component to the second polymer component, (c) total polymer content, (d) molecular weights of the first and/or second polymer components, or (e) combinations thereof.

In some embodiments, crosslinks that form during the transition of the precursor state to the polymer network state do not comprise covalent crosslinks.

In many embodiments, such a polymer combination preparation is temperature-responsive. In some such embodiments, such a polymer combination preparation is characterized in that it transitions from a precursor state to a polymer network state in response to a temperature at or above CGT. For example, in some embodiments, the CGT for a provided polymer combination preparation is 18-39° C. In some embodiments the CGT for a provided polymer combination preparation is room temperature. In some embodiments, the CGT for a provided polymer combination preparation is 20-25° C. In some embodiments, the CGT for a provided polymer combination preparation is 25-30° C. In some embodiments, the CGT for a provided polymer combination preparation is 30-35° C. In some embodiments the CGT for the polymer combination preparation is body temperature of a subject.

While many different poloxamers may be used in provided polymer combination preparations, in some embodiments, certain poloxamers, e.g., Poloxamer 407 (P407), Poloxamer 338 (P338), or Poloxamer 188 (P188) are particularly useful in certain polymer combination preparations described herein. For example, in some embodiments, a poloxamer included as a first polymer component in a polymer combination preparation described herein is or comprises P407. In some embodiments, a first polymer component (e.g., comprising P407) is present in a provided polymer combination preparation at a concentration of 4% (w/w) to 12.5% (w/w), or 4% (w/w) to 11% (w/w), or 4% (w/w) to 10.5% (w/w), or 4% (w/w) to 10% (w/w). In some embodiments, a first polymer component (e.g., comprising P407) is present in a provided polymer combination preparation at a concentration of 5% (w/w) to 12.5% (w/w), or 5% (w/w) to 11% (w/w), or 5% (w/w) to 10.5% (w/w), or 5% (w/w) to 10% (w/w). In some embodiments, a first polymer component (e.g., comprising P407) is present in a provided polymer combination preparation at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a polymer combination preparation described herein comprises a total polymer content of at least 6% (w/w), at least 8% (w/w), at least 10% (w/w), at least 12%, or at least 15% (w/w). In some embodiments, a polymer combination preparation described herein comprises a total polymer content of 6% (w/w) to 20% (w/w), or 6% (w/w) to 15% (w/w), or 7% (w/w) to 15% (w/w). In some embodiments, a polymer combination preparation described herein comprises a total polymer content of 8% (w/w) to 20% (w/w), or 8% (w/w) to 15% (w/w), or 10% (w/w) to 15% (w/w).

In some embodiments, a polymer combination preparation described herein is characterized by a weight ratio of a first polymer component to a second polymer component of 1:1 to 14:1, or 1:1 to 10:1. In some embodiments, a polymer combination preparation described herein is characterized by a weight ratio of a first polymer component to a second polymer component of 1:1 to 1:3 or 1:1 to 1:2. In some embodiments, a polymer combination preparation described herein is characterized by a weight ratio of a first polymer component to a second polymer component of 1:1 to 22:1, or 1:1 to 18:1.

In some embodiments, a second polymer component in a provided polymer combination preparation is or comprises a carbohydrate polymer. Examples of a carbohydrate polymer that may be useful in accordance with the present disclosure include, but are not limited to, hyaluronic acid, chitosan, alginate, and variants and combinations thereof. In some embodiments, a carbohydrate polymer in a provided polymer combination preparation may be present at a concentration of below about 5% (w/w). In some embodiments, a carbohydrate polymer in a provided polymer combination preparation may be present at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

In some embodiments, a carbohydrate polymer that is useful for certain polymer combination preparations described herein is or comprises hyaluronic acid. In some embodiments, such hyaluronic acid may have an average molecular weight of 50 kDa to 2 MDa. In some embodiments, such hyaluronic acid may have an average molecular weight of 100 kDa to 500 kDa. In some embodiments, such hyaluronic acid may have an average molecular weight of 125 kDa to 375 kDa. In some embodiments, such hyaluronic acid may have an average molecular weight of 100 kDa to 400 kDa. In some embodiments, such hyaluronic acid may have an average molecular weight of 500 kDa to 1.5 MDa. In some embodiments, molecular weight of hyaluronic acid is characterized by weight average molecular weight. In some embodiments, molecular weight of hyaluronic acid is characterized by viscosity average molecular weight, which in some embodiments can be determined by converting intrinsic viscosity of hyaluronic acid to average molecular weight, for example, using the Mark-Houwink Equation. In some embodiments, molecular weight of hyaluronic acid can be measured by Size Exclusion Chromatography-Multiple Angle Laser Light Scattering (SEC-MALLS).

In some embodiments, number average molecular weight (Mn), weight average molecular weight (Mw), and/or dispersity (as characterized by polydispersity index) can be determined using SEC-MALLS.

In some embodiments, a carbohydrate polymer that is useful for certain polymer combination preparations described herein is or comprises a chitosan or a modified chitosan. In some embodiments, an exemplary modified chitosan is or comprises carboxymethyl chitosan.

In some embodiments, a preparation or composition comprising a polymer combination preparation as utilized and/or described herein in a precursor state. In some embodiments, a preparation or composition comprising a polymer combination preparation as utilized and/or described herein in a polymer network state (e.g., having one or more characteristics as described herein).

In some embodiments, a polymer network state is or comprises a viscous solution or colloid. In some embodiments, such a polymer network state may be characterized by a storage modulus of 100 Pa to 500 Pa. In some embodiments, a polymer network state is or comprises a hydrogel. In some embodiments, such a polymer network state may be characterized by a storage modulus of 500 Pa to 10,000 Pa, or 750 Pa to 7500 Pa.

In some embodiments, a polymer network state of a provided polymer combination preparation is characterized by a storage modulus that is at least 40% lower than that of a hydrogel formed from a P407 solution at a concentration of 18% (w/w). In some embodiments, a polymer network state of a provided polymer combination preparation, which its precursor state has been stored at a temperature that is below CGT (e.g., 2-8° C.) over a period of 1 month or longer, is characterized by a storage modulus, for example, as measured at 37° C., that maintains substantially the same (e.g., within 20%, within 10%, within 5%, or lower), as compared to that of a polymer network formed from a precursor state of such a provided polymer combination preparation that is freshly prepared. As will be understood by those skilled in the art, storage modulus of a biomaterial may be affected by biodegradation, chemical degradation (e.g., oxidation), and/or phase separation of polymer components in a combination.

In some embodiments, a polymer combination preparation as described and/or utilized herein has pH 5.0-8.5. In some embodiments, a polymer combination preparation as described and/or utilized herein has pH 7-8 (e.g., pH 7.4). For example, in some embodiments, a precursor state of a polymer combination preparation is a solution of the polymer combination preparation in a solvent system having pH 5.0-8.5 (e.g., in some embodiments pH 7-8). In some embodiments, such a solvent system is a buffered system. In some embodiments, such a buffered system may comprise one or more salts (e.g., but not limited to sodium phosphate, and/or sodium hydrogen carbonate). In some embodiments, such a solvent system is a buffer system having a higher buffering capacity than a 10 mM phosphate buffer. In some embodiments, such a solvent system is a buffer system having a higher buffering capacity than a 20 mM phosphate buffer.

In some embodiments, a preparation or composition described herein may comprise a polymer combination preparation (e.g., ones described herein) and resiquimod, e.g., for treatment of a disease, disorder, or a condition (e.g., cancer). In some embodiments, such a polymer combination preparation is characterized in that a test animal group with spontaneous metastases having, at a tumor resection site, the polymer combination preparation in the polymer network state has a higher percent survival than a comparable test animal group having, at a tumor resection site, a polymer combination preparation without the immunomodulatory payload, as assessed at 2 months or 3 months after the administration.

### I. Compositions or Preparations Comprising Provided Polymer Combination Preparations

In some embodiments, the present disclosure, among other things, provides compositions and/or preparations comprising polymer combination preparations (e.g., ones described herein) that are temperature-responsive, which thus permit in situ gelation at a target site in the absence of crosslinking treatments (e.g., introduction of UV radiation and/or chemical crosslinkers) that may have toxic or otherwise damaging effects for the recipient and/or for a payload that may be included in or with a biomaterial.

In some embodiments, the present disclosure provides compositions comprising certain polymer combination preparations that are useful to provide a sustained release of payloads (e.g., resiquimod) incorporated in polymer combination preparations. For example, in some embodiments, certain compositions and/or preparations described herein can be remarkably useful when such compositions incorporating one or more immunomodulatory payloads (e.g., resiquimod) are administered to subjects who have undergone or are undergoing a tumor resection. By way of example only, in some embodiments, a composition or preparation of the present disclosure may comprise at least one innate immunity modulatory payload (e.g., at least resiquimod). In some embodiments, a composition or preparation of the present disclosure may comprise at least one innate immunity modulatory payload and at least one adaptive immunity modulatory payload. In some embodiments, a composition or preparation of the present disclosure may comprise at least one innate immunity modulatory payload, at least one adaptive immunity modulatory payload, and at least one immunomodulatory cytokine. In some embodiments, a composition or preparation of the present disclosure may comprise at least one inhibitor of a proinflammatory immune response.

In some embodiments, the present disclosure provides compositions comprising certain polymer combination preparations that by themselves are sufficient to provide an immunomodulatory response (e.g., to provide sufficient innate immunity agonism) to achieve beneficial effects even absent a separate immunomodulatory payload (e.g., resiquimod).

In some embodiments, a polymer combination preparation described herein is characterized in that it forms a polymer network; without wishing to be bound by any particular theory, it is noted that, in some embodiments, such a network may act as a scaffold or depot for a payload (e.g., for an immunomodulatory payload, e.g., resiquimod) within a polymer combination preparation.

In some embodiments, a polymer combination preparation comprising a biomaterial preparation and a payload agent (e.g., an immunomodulatory payload, e.g., resiquimod) may perform as an extended release formulation, for example, in that the payload is released from the composition more slowly (i.e. over a longer period of time) than is observed for an otherwise comparable composition lacking the polymer combination preparation (e.g., lacking one or all polymer components thereof).

In some embodiments, a polymer combination preparation for use as described herein comprises one or more polymers (e.g., ones described herein). In certain embodiments, a polymer combination preparation may comprise one or more positively charged polymers. In some embodiments, a polymer combination preparation for use as described herein may comprise one or more negatively charged polymers. In some embodiments, a polymer combination preparation for use as described herein may comprise one or more neutral polymers.

### Provided Polymer Combination Preparations

In some embodiments, the present disclosure, among other things, provides polymer combination preparations comprising at least first and second polymer components, wherein the first polymer component is or comprises a poloxamer (e.g., ones described herein) and the second polymer component is not a poloxamer, wherein the first polymer component is present in the polymer combination preparation at a concentration of 12.5% (w/w) or below. In some embodiments, such polymer combination preparation is characterized in that it transitions from a precursor state to a polymer network state in response to a gelation trigger, which is or comprises one or more of the following: (a) temperature at or above critical gelation temperature (CGT) for the polymer combination preparation, (b) critical gelation weight ratio of the first polymer component to the second polymer component, (c) total polymer content, (d) molecular weights of the first and/or second polymer components, or (e) combinations thereof. A polymer network state of a provided polymer combination preparation has a viscosity materially above that of the precursor state and comprises crosslinks not present in the precursor state. In some embodiments, a precursor state of a provided polymer combination preparation is a liquid state. In some embodiments, a precursor state of a provided polymer combination preparation is an injectable state. In some embodiments, a polymer network state of a provided polymer combination preparation is a more viscous liquid state. In some embodiments, a polymer network state of a provided polymer combination preparation is a hydrogel.

In some embodiments, a provided polymer combination preparation is temperature-responsive, so that, e.g., its gelation (e.g., its transition from a liquid state to a gelled state) can occur upon exposure to a particular temperature. In many such embodiments, exposure to body temperature (e.g., by application to a site) is sufficient to trigger such gelation; in some embodiments, additional warmth may be applied. By way of example only, in some embodiments, a temperature-responsive polymer combination preparation as described herein is characterized in that it transitions from a precursor state (e.g., a liquid state or an injectable state) to a polymer network state that has a viscosity and/or storage modulus materially above that of the precursor state (e.g., a more viscous state or a hydrogel) when such a polymer combination preparation is exposed to a gelation trigger, which is or comprises a temperature at or above critical gelation temperature (CGT) for the polymer combination preparation. In some embodiments, a CGT for a provided polymer combination preparation is at least 10° C. or higher, including, e.g., at least 10° C., at least 11° C., at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., 33° C., at least 34° C., at least 35° C., at least 36° C., at least 37° C., at least 38° C., at least 39° C., at least 40° C., or higher. In some embodiments, a CGT for a provided polymer combination preparation is about 10° C. to about 15° C. In some embodiments, a CGT for a provided polymer combination preparation is about 12° C. to about 17° C. In some embodiments, a CGT for a provided polymer combination preparation is about 14° C. to about 19° C. In some embodiments, a CGT for a provided polymer combination preparation is about 16° C. to about 21° C. In some embodiments, a CGT for a provided polymer combination preparation is about 18° C. to about 23° C. In some embodiments, a CGT for a provided polymer combination preparation is about 20° C. to about 25° C. In some embodiments, a CGT for a provided polymer combination preparation is about 22° C. to about 27° C. In some embodiments, a CGT for a provided polymer combination preparation is about 24° C. to about 29° C. In some embodiments, a CGT for a provided polymer combination preparation is about 26° C. to about 31° C. In some embodiments, a CGT for a provided polymer combination preparation is about 28° C. to about 33° C. In some embodiments, a CGT for a provided polymer combination preparation is about 30° C. to about 35° C. In some embodiments, a CGT for a provided polymer combination preparation is about 32° C. to about 37° C. In some embodiments, a CGT for a provided polymer combination preparation is about 34° C. to about 39° C. In some embodiments, a CGT for a provided polymer combination preparation is about 35° C. to about 39° C. In some embodiments, a CGT for a provided polymer combination preparation is at or near physiological temperature of a subject (e.g., a human subject) receiving such a polymer combination preparation.

In some embodiments, a provided polymer combination preparation is temperature-reversible. For example, in some embodiments, a provided polymer combination preparation is characterized in that it transitions from a precursor state (e.g., a liquid state or an injectable state) to a polymer network state that has a viscosity and/or storage modulus materially above that of the precursor state (e.g., a more viscous state or a hydrogel) when such a polymer combination preparation is exposed to a temperature at or above critical gelation temperature (CGT) for the polymer combination preparation; and it may revert from the polymer network state to a state that has a viscosity and/or storage modulus materially lower than that of the polymer network state (e.g., a liquid state or original state of a provided polymer combination preparation).

In some embodiments, a polymer combination preparation described herein does not comprise a chemical crosslinker. Those of skill in the art will appreciate that, in some embodiments, a chemical crosslinker is characterized in that it facilitates formation of covalent crosslinks between polymer chains. In some embodiments, a chemical crosslinker is or comprises a small-molecule crosslinker, which can be derived from a natural source or synthesized. Non-limiting examples of small-molecule crosslinkers include genipin, dialdehyde, glutaraldehyde, glyoxal, diisocyanate, glutaric acid, succinic acid, adipic acid, acrylic acid, diacrylate, etc.). In some embodiments, a chemical crosslinker may involve crosslinking using thiols (e.g., EXTRACEL®, HYSTEM®), methacrylates, hexadecylamides (e.g., HYMOVIS®), and/or tyramines (e.g., CORGEL®). In some embodiments, a chemical crosslinker may involve crosslinking using formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). Accordingly, in some embodiments, crosslinks that form during the transition from a precursor state to a polymer network state do comprise covalent crosslinks.

In some embodiments, a first polymer component (e.g., poloxamer described herein) and a second polymer component (e.g., ones described herein) are present in a polymer combination preparation at a critical gelation weight ratio of 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 12:1; 13:1, 14:1, 15:1, 16:1, 17:1, 18:1, 19:1, or 20:1. In some embodiments, a first polymer component (e.g., poloxamer described herein) and a second polymer component (e.g., ones described herein) are present in a polymer combination preparation at a critical gelation weight ratio of 1:1 to 20:1 or 1:1 to 18:1, or 1:1 to 14:1, or 1.5:1 to 14:1, or 2:1 to 13:1, or 1:1 to 10:1, or 2:1 to 20:1, or 2:1 to 18:1, or 2:1 to 10:1. In some embodiments, a first polymer component (e.g., poloxamer described herein) and a second polymer component (e.g., ones described herein) are present in a polymer combination preparation at a critical gelation weight ratio of 1:1 to 10:1. In some embodiments, a first polymer component (e.g., poloxamer described herein) and a second polymer component (e.g., ones described herein) are present in a polymer combination preparation at a critical gelation weight ratio of 2:1 to 10:1. In some embodiments, a first polymer component (e.g., poloxamer described herein) and a second polymer component (e.g., ones described herein) are present in a polymer combination preparation at a critical gelation weight ratio such that the second polymer component may be in a greater amount by weight than that of the first polymer component. For example, in some embodiments, a first polymer component (e.g., poloxamer described herein) and a second polymer component (e.g., ones described herein) are present in a polymer combination preparation at a critical gelation weight ratio of 1:1.1, 1:1.2, 1:1.3, 1:1.4, 1:1.5, 1:1.6, 1:1.7, 1:1.8, 1:1.9, 1:2, etc. In some such embodiments, poloxamer concentration may be less than 7% (w/w) or lower, e.g., 6% (w/w), 5% (w/w), 4% (w/w), or lower.

In some embodiments, a polymer combination preparation provided herein comprising at least first and second polymer components (e.g., ones described herein) may comprise at least one additional polymer components, including, e.g., at least one, at least two, at least three, at least four, at least five, at least six, or more additional polymer components, which in some embodiments may be or comprise a biocompatible and/or biodegradable polymer component (e.g., as described herein).

In some embodiments, a provided polymer combination preparation comprises total polymer content of at least 5% (w/w) or higher, including, e.g., at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), at least 20% (w/w), or higher. In some embodiments, a provided polymer combination preparation comprises total polymer content of 5% (w/w) to 20% (w/w), or 6% (w/w) to 18% (w/w), or 8% (w/w) to 15% (w/w), or 9% (w/w) to 12% (w/w). In some embodiments, a polymer combination preparation described herein comprises a total polymer content of 6% (w/w) to 20% (w/w), or 8% (w/w) to 20% (w/w), or 10% (w/w) to 15% (w/w).

In some embodiments, a first polymer component, which is or comprises a poloxamer, is present in a provided polymer combination preparation at a concentration of no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w), no more than 7% (w/w), no more than 6% (w/w), no more than 5% (w/w), or no more than 4% (w/w)). In some embodiments, a first polymer component, which is or comprises a poloxamer, is present in a provided polymer combination preparation at a concentration of 5% (w/w) to 12.5% (w/w), or 8% (w/w) to 12.5% (w/w), or 5% (w/w) to 11% (w/w), or 5% (w/w) to 10% (w/w), or 6% (w/w) to 10% (w/w), or 8% (w/w) to 10% (w/w). In some embodiments, a first polymer component, which is or comprises a poloxamer, is present in a provided polymer combination preparation at a concentration of 4% (w/w) to 12.5% (w/w), or 4% (w/w) to 11% (w/w), or 4% (w/w) to 10.5% (w/w), or 4% (w/w) to 10% (w/w). In some embodiments, a first polymer component, which is or comprises a poloxamer, is present in a provided polymer combination preparation at a concentration of 5% (w/w) to 12.5% (w/w), or 5% (w/w) to 11% (w/w), or 5% (w/w) to 10.5% (w/w), or 5% (w/w) to 10% (w/w). In some embodiments, a first polymer component, which is or comprises a poloxamer, is present in a provided polymer combination preparation at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a second polymer component may be present in a provided polymer combination preparation at a concentration of no more than 15% (w/w). In some embodiments, a second polymer component may be present in a provided polymer combination preparation at a concentration of no more than 10% (w/w), including, e.g., at a concentration of 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a second polymer component may be present in a provided polymer combination preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), or higher. In some embodiments, a second polymer component in a provided polymer combination preparation may be present at a concentration of 0.1% (w/w) to 10% (w/w), or 0.1% (w/w) to 8% (w/w), or 0.1% (w/w) to 5% (w/w), or 1% (w/w) to 5% (w/w). In some embodiments, a second polymer component in a provided polymer combination preparation may be present at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

### A. First Polymer Component Comprising One or More Exemplary Poloxamers and Variants Thereof

In some embodiments, a provided polymer combination preparation comprises a poloxamer or a variant thereof. Poloxamer is typically a block copolymer comprising a hydrophobic chain of polyoxypropylene (e.g., polypropylene glycol, PPG, and/or poly(propylene oxide), PPO) flanked by two hydrophilic chains of polyoxyethylene (e.g., polyethylene glycol, PEG, and/or poly(ethylene oxide), PEO). Poloxamers are known by the trade names Synperonic, Pluronic, and/or Kolliphor. Generally, poloxamers are non-ionic surfactants, which in some embodiments may have a good solubilizing capacity, low toxicity, and/or high compatibility with cells, body fluids, and a wide range of chemicals.

In some embodiments, a poloxamer for use in accordance with the present disclosure may be a poloxamer known in the art. For example, as will be understood by a skilled person in the art, poloxamers are commonly named with the letter P (for poloxamer) followed by three digits: the first two digits multiplied by 100 give the approximate molecular mass of the polyoxypropylene chain, and the last digit multiplied by 10 gives the percentage polyoxyethylene content. By way of example only, P407 refers to a poloxamer with a polyoxypropylene molecular mass of 4,000 g/mol and a 70% polyoxyethylene content). A skilled person in the art will also understand that for the Pluronic and Synperonic tradenames, coding of such poloxamers starts with a letter to define its physical form at room temperature (e.g., L=liquid, P=paste, F=flake (solid)) followed by two or three digits, wherein the first digit (two digits in a three-digit number) in the numerical designation, multiplied by 300, indicates the approximate molecular weight of the polyoxypropylene chain; and the last digit, multiplied by 10, gives the percentage polyoxyethylene content. By way of example only, L61 refers to a liquid preparation of poloxamer with a polyoxypropylene molecular mass of 1,800 g/mol and a 10% polyoxyethylene content. In addition, as will be apparent to a skilled artisan, poloxamer 181 (P181) is equivalent to Pluronic L61 and Synperonic PE/L61.

In some embodiments, a poloxamer that may be included in a polymer combination preparation described herein may be or comprise Poloxamer 124 (e.g., Pluronic L44 NF), Poloxamer 188 (e.g., Pluronic F68NF), Poloxamer 181 (e.g., Pluronic L61), Poloxamer 182 (e.g., Pluronic L62), Poloxamer 184 (e.g., Pluronic L64), Poloxamer 237 (e.g., Pluronic F87 NF), Poloxamer 338 (e.g., Pluronic F108 NF), Poloxamer 331 (e.g., Pluronic L101), Poloxamer 407 (e.g., Pluronic F127 NF), or combinations thereof. In some embodiments, a provided polymer combination preparation can comprise at least two or more different poloxamers. Additional poloxamers as described in Table 1 of Russo and Villa “Poloxamer Hydrogels for Biomedical Applications” Pharmaceutics (2019) 11(12):671, the contents of which are incorporated herein by reference for the purposes described herein, may be also useful for polymer combination preparations described herein.

In some embodiments, a poloxamer that may be included in a polymer combination preparation described herein may be or comprise Poloxamer 407 (P407). In some embodiments, P407 is a triblock poloxamer copolymer having a hydrophobic PPO block flanked by two hydrophilic PEO blocks. The approximate length of the two PEO blocks is typically 101 repeat units, while the approximate length of the PPO block is 56 repeat units. In some embodiments, P407 has an average molecular weight of approximately 12,600 Da of which approximately 70% corresponds to PEO. In some embodiments, P407 can readily self-assemble to form micelles dependent upon concentration and ambient temperature. Without wishing to be bound by a particular theory, dehydration of hydrophobic PPO blocks combined with hydration of PEO blocks may lead to formation of spherical micelles, and subsequent packing of the micellar structure results in a 3D cubic lattice that constitutes the main structure of poloxamer hydrogels. They are also biodegradable, non-toxic, and stable, and are therefore suitable for use as controlled release of therapeutic agents. As appreciated by one of ordinary skill in the art, P407 concentrations in hydrogel formulations based on binary poloxamer/water mixtures are typically in the range from 16-20% w/v, with a value of approximately 18% w/v most frequently used. See, e.g., Pereia et al. “Formulation and Characterization of Poloxamer 407@: Thermoreversible Gel Containing Polymeric Microparticles and Hyaluronic Acid” Quim. Nova, Vol. 36, No. 8, 1121-1125 (2013), the contents of which are incorporated herein by reference in their entirety.

Various crosslinking approaches, e.g., chemical crosslinking and enzyme-mediated crosslinking approaches, were used to crosslink P407 alone or in combination with another polymer at a P407 concentration lower than a typical range of 16-20% w/v. See, e.g., Ryu et al. “Catechol-functionalized chitosan/pluronic hydrogels for tissue adhesives and hemostatic materials” Biomacromolecules (2011) 12(7): 2653-2659; Lee et al. “Thermo-sensitive, injectable, and tissue adhesive sol-gel transition hyaluronic acid/pluronic composite hydrogels prepared from bio-inspired catechol-thiol reaction” Soft Matter (2010) 6: 977-983; and Chung et al. “Thermo-sensitive biodegradable hydrogels based on stereocomplexed pluronic multi-block copolymers for controlled protein delivery” J Control Release (2008) 127: 22-30; and Lee et al. “Enzyme-mediated cross-linking of pluronic copolymer micelles for injectable and in situ forming hydrogels” Acta Biomater (2011) 7: 1468-76, the contents of each of which are incorporated by reference in their entirety. However, in some embodiments, such crosslinking approaches require use of a chemical crosslinker or an enzyme, and/or a modified P407, which may not be desirable for in vivo administration. In some embodiments, the present disclosure, among other things, provides an insight that certain polymer combination preparations (e.g., ones described herein) may be particularly useful to form temperature-responsive hydrogels in the absence of chemical crosslinks or enzyme-mediated crosslinks, while the concentration of P407 is no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w)) in the polymer combination preparation. In some embodiments, the concentration of P407 is present in a provided polymer combination at a concentration of 6% (w/w) to 12.5% (w/w), or 6% (w/w) to 11% (w/w), 5% (w/w) to 12.5% (w/w), or 5% (w/w) to 11% (w/w), or 8% (w/w) to 12.5% (w/w), or 5% (w/w) to 10% (w/w), or 8% (w/w) to 10% (w/w) or 6% (w/w) to 10% (w/w). In some embodiments, the concentration of P407 is present in a provided polymer combination at a concentration of 4% (w/w) to 12.5% (w/w), or 4% (w/w) to 11% (w/w), or 4% (w/w) to 10.5% (w/w), or 4% (w/w) to 10% (w/w). In some embodiments, the concentration of P407 is present in a provided polymer combination at a concentration of 5% (w/w) to 12.5% (w/w), or 5% (w/w) to 11% (w/w), or 5% (w/w) to 10.5% (w/w), or 5% (w/w) to 10% (w/w). In some embodiments, the concentration of P407 is present in a provided polymer combination at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a P407 that may be included in a polymer combination preparation described herein may be or comprise compendial poloxamer 407. In some embodiments, such compendial poloxamer 407 included in a provided polymer combination preparation has not undergone additional purification steps. In some embodiments, such compendial poloxamer 407 included in a provided polymer combination preparation has not been modified, e.g., in some embodiments genetically modified. In some embodiments, a P407 that may be useful in a polymer combination preparation described herein may have a sol-to-gel transition temperature (T_{sol-gel}) in PBS of at least 18° C. or higher, including, e.g., 18.5° C., 19° C., 19.5° C., 20° C., 20.5° C., 21° C., 21.5° C., 22° C., 22.5° C., 23° C., or 23.5° C. In some embodiments, a P407 that may be useful in a polymer combination preparation described herein may have an average molecule weight of no more than 12 kDa, e.g., no more than 11.5 kDa, no more than 11 kDa, no more than 10.5 kDa, or lower. As understood by one of ordinary skill in the art, the T_{sol-gel} and/or average molecule weight of P407 in PBS may be varied by purification. For example, in some embodiments, the T_{sol-gel} and/or average molecule weight of P407 in PBS may increase when low molecular weight copolymer molecules and/or impurities are removed from compendial P407. Alternatively, the T_{sol-gel} and/or average molecule weight of P407 in PBS may decrease when high molecular weight copolymer molecules and/or impurities are removed from compendial P407. See, e.g., Fakhari et al. “Thermogelling properties of purified poloxamer 407” Heliyon (2017) 3(8):e00390, the contents of which are incorporated herein by reference in their entirety.

In some embodiments, a P407 to be included in a polymer combination preparation described herein may be a non-conjugated or non-modified P407 (e.g., a P407 that is not covalently conjugated to a moiety, such as, e.g., a polymer or an amino acid). Examples of conjugated P407 include, but are not limited to grafting P407 onto a carbohydrate polymer, e.g., chitosan, or a thiolated P407). See, e.g., Park et al. “Thermosensitive chitosan-Pluronic hydrogel as an injectable cell delivery carrier for cartilage regeneration” Acta Biomaterialia (2009) 5(6): 1956-1965; and Ryu et al. “Catechol-functionalized chitosan/pluronic hydrogels for tissue adhesives and hemostatic materials” Biomacromolecules (2011) 12(7): 2653-2659, the contents of each of which are incorporated herein by reference in their entirety.

In some embodiments, a poloxamer that may be included in a polymer combination preparation described herein may be or comprise Poloxamer 338.

In some embodiments, a poloxamer that may be included in a polymer combination preparation described herein may be or comprise Poloxamer 331.

In some embodiments, a poloxamer that may be included in a polymer combination preparation described herein may be or comprise Poloxamer 237.

In some embodiments, a poloxamer that may be included in a polymer combination preparation described herein may be or comprise Poloxamer 188.

In some embodiments, a poloxamer that may be included in a polymer combination preparation described herein may be or comprise Poloxamer 184.

In some embodiments, a poloxamer that may be included in a polymer combination preparation described herein may be or comprise Poloxamer 182.

In some embodiments, a poloxamer that may be included in a polymer combination preparation described herein may be or comprise Poloxamer 181.

In some embodiments, a poloxamer that may be included in a polymer combination preparation described herein may be or comprise Poloxamer 124.

In some embodiments, a poloxamer that may be included in a polymer combination preparation described herein may have a polyoxyethylene content of at least 30% by weight, including, e.g., at least 40%, at least 50%, at least 60%, at least 70%, at least 80%, at least 90%, or higher by weight. In some embodiments, a poloxamer may have a polyoxyethylene content of 50-90% by weight. In some embodiments, a poloxamer has a polyoxyethylene content of 60-90%. In some embodiments, a poloxamer has a polyoxyethylene content of 70-90%. In some embodiments, a poloxamer has a polyoxyethylene content of about 70%. In some embodiments, a poloxamer has a polyoxyethylene content of about 80%.

In some embodiments, a poloxamer that may be included in a polymer combination preparation described herein may have an average molecular weight of at least 1,500 g/mol or higher, including, e.g., at least 2,000 g/mol, at least 2,500 g/ml, at least 3,000 g/mol, at least 4,000 g/mol, at least 5,000 g/mol, at least 6,000 g/mol, at least 7,000 g/mol, at least 8,000 g/mol, at least 9,000 g/mol, at least 10,000 g/mol, at least 11,000 g/mol, at least 12,000 g/mol, at least 13,000 g/mol, at least 14,000 g/mol, at least 15,000 g/mol, at least 16,000 g/mol, at least 17,000 g/mol, at least 18,000 g/mol, at least 19,000 g/mol, at least 20,000 g/mol, or higher. In some embodiments, a poloxamer may have an average molecular weight between about 1,500 and 20,000 g/mol. In some embodiments, a poloxamer may have an average molecular weight between about 4,000 and 12,000 g/mol. In some embodiments, a poloxamer may have an average molecular weight between about 5,000 and 15,000 g/mol, or between 9,000 and 15,000 g/mol, or between 10,000 and 15,000 g/mol, or between about 11,000 and 14,000 g/mol, or between about 11,500 and 13,000 g/mol, or between about 12,000 and 13,000 g/mol, or between about 6,000 and 10,000 g/mol, or between about 7,000 and 9,000 g/mol, or between about 7,500 and 8,500 g/mol. In some embodiments, a poloxamer may have an average molecular weight between 9,500 and 15,000 g/mol. In some embodiments, a poloxamer may have an average molecular weight between 6,000 and 10,000 g/mol. In some embodiments, a poloxamer may have an average molecular weight between 12,000 and 18,000 g/mol. In some embodiments, a poloxamer may have an average molecular weight between 1,500 and 3,000 g/mol. In some embodiments, a poloxamer may have an average molecular weight between 6,000 and 9,000 g/mol. A skilled practitioner will understand that an average molecular weight described herein can be a number average molecular weight, a viscosity average molecular weight, or a weight average molecular weight. In some embodiments, polymers described herein (e.g., poloxamers and other polymers described herein) are characterized by weight average molecular weight. In some embodiments, polymers described herein (e.g., hyaluronic acid described herein) are characterized by viscosity average molecular weight, which in some embodiments can be determined by converting intrinsic viscosity measurement to average molecular weight, for example, using the Mark-Houwink equation.

In some embodiments, a poloxamer that may be included in a polymer combination preparation described herein may have polyoxypropylene with an average molecular weight between 1,000 and 5,000 g/mol, or between 1,500 and 4,500 g/mol.

In some embodiments, a poloxamer that may be included in a polymer combination preparation described herein may be a poloxamer variant. Examples of a poloxamer variant include, but are not limited to, poloxamines (e.g., amphiphilic block copolymers formed by four arms of poly(ethylene oxide)-poly(propylene oxide) (PEO-PPO) blocks bonded to a central ethylenediamine moiety), acrylate-modified poloxamer, thiol-modified poloxamer, and combinations thereof. See, e.g., Niu et al., J. Controlled Release, 2009, 137:49-56; and Alvarex-Lorenzo et al. “Poloxamine-based nanomaterials for drug delivery” Frontiers in Bioscience (2010), the contents of each of which are hereby incorporated by reference for at least their disclosure on modified poloxamers.

B. Second Polymer Component Comprising One or More Exemplary Polymers that are not Poloxamers

In some embodiments, a polymer combination preparation described herein may comprise at least two polymer components, including, e.g., at least three, at least four, at least five, or more polymer components. In some embodiments, a second polymer component of a provided polymer combination preparation comprising poloxamer as a first polymer component at a concentration of 12.5% (w/w) or below may be or comprise at least one, including, e.g., at least two, at least three, at least four or more, biocompatible and/or biodegradable polymer components. Examples of such a biocompatible and/or biodegradable polymer component include, but are not limited to immunomodulatory polymers, carbohydrate polymers (e.g., a polymer that is or comprises a carbohydrate, e.g., a carbohydrate backbone, including, e.g., but not limited to chitosan, alginate, hyaluronic acid, and/or variants thereof), polyacrylic acid, silica gels, polyethylenimine (PEI), polyphosphazene, and/or variants thereof), cellulose, chitin, chondroitin sulfate, collagen, dextran, gelatin, ethylene-vinyl acetate (EVA), fibrin, poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycarboxybetaine (PCB), polysulfobetaine (PSB), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, starch, variants thereof, and/or combinations thereof.

In some embodiments, a second polymer component of a provided polymer combination preparation is or comprises a non-ionic polymer component. Examples of such a non-ionic polymer component include, but are not limited to polyvinyl alcohol (PVA), polyethylene oxide (PEO), and combinations thereof. In some embodiments, a second polymer component of a provided polymer combination preparation is or comprises a cationic polymer component, e.g., but not limited to chitosan, amino-containing polymers, collagen, gelatin, and combinations thereof. In some embodiments, a second polymer component of a provided polymer combination preparation is or comprises an anionic polymer component, examples of which may include, but are not limited to alginate, gellan gum, pectin, xanthan gum, carboxymethyl cellulose (CMC), polyacrylic acid, polyaspartic acid, and combinations thereof.

In some embodiments, a second polymer component of a provided polymer combination preparation is or comprises an immunomodulatory polymer, e.g., a polymer that modulates one or more aspects of an immune response (e.g., a polymer that induces innate immunity agonism). In some embodiments, an immunomodulatory polymer may be or comprise a polymer agonist of innate immunity as described in International Patent Application No. PCT/US20/31169 filed May 1, 2020, (published as WO2020/223698A1). In some embodiments, an immunomodulatory polymer may be or comprise a carbohydrate polymer (e.g., a polymer that is or comprises a carbohydrate, e.g., a carbohydrate backbone, including, e.g., but not limited to chitosan, alginate, hyaluronic acid, and/or variants thereof).

In some embodiments, a provided polymer combination preparation comprises at least one poloxamer at a concentration of 12.5% or below (e.g., 11% (w/w), 10.5% (w/w), 10% (w/w), 9% (w/w), 8% (w/w), 7% (w/w), 6% (w/w), or lower) and a second polymer component, which may be or comprise a carbohydrate polymer, e.g., a polymer that is or comprises a carbohydrate, e.g., a carbohydrate backbone, including, e.g., but not limited to hyaluronic acid, chitosan, and/or variants thereof.

### (i) Exemplary Hyaluronic Acid and Variants Thereof

In some embodiments, a carbohydrate polymer included in a provided polymer combination preparation comprising poloxamer is or comprises hyaluronic acid or a variant thereof. Hyaluronic acid (HA), also known as hyaluronan or hyaluronate, is a non-sulfated member of a class of polymers known as glycosaminoglycans (GAG) that is widely distributed in body tissues. HA is found as an extracellular matrix component of tissue that forms a pericellular coat on the surfaces of cells. In some embodiments, HA is a polysaccharide (which in some embodiments may be present as a salt, e.g., a sodium salt, a potassium salt, and/or a calcium salt) having a molecular formula of (C₁₄H₂₁NO₁₁)ₙ where n can vary according to the source, isolation procedure, and/or method of determination.

In some embodiments, HA that may be useful in accordance with the present disclosure can be isolated or derived from many natural sources. For example, in some embodiments, HA can be isolated or derived from, including, e.g., human umbilical cord, rooster combs, and/or connective matrices of vertebrate organisms. In some embodiments, HA can be isolated or derived from a capsular component of bacteria such as Streptococci. See, e.g., Kendall et al, (1937), Biochem. Biophys. Acta, 279, 401-405. In some embodiments, HA and/or variants thereof can be produced via microbial fermentation. In some embodiments, HA and/or variants thereof may be a recombinant HA or variants thereof, for example, produced using Gram-positive and/or Gram-negative bacteria as a host, including, e.g., but not limited to Bacillus sp., Lactococcos lactis, Agrobacterium sp., and/or Escherichia coli.

As discussed in the International Patent Application No. PCT/US20/31169 filed May 1, 2020 (published as WO2020/223698A1), biological activities of HA differ, depending on its molecular weight—for example, high molecular weight HA (high MW HA) can possess anti-inflammatory or immunosuppressive activities, while low molecular weight HA (low MW HA) may exhibit pro-inflammatory or immunostimulatory behaviors. See, e.g., Gao et al. “A low molecular weight hyaluronic acid derivative accelerates excisional wound healing by modulating pro-inflammation, promoting epithelialization and neovascularization, and remodeling collagen” Int. J Mol Sci (2019) 20:3722; Cyphert et al. “Size Matters: Molecular Weight Specificity of Hyaluronan Effects in Cell Biology.” Int. J. Cell Biol. (2015) 2015: 563818; Dicker et al. “Hyaluronan: A simple polysaccharide with diverse biological functions” Acta Biomater. (2014) 10:1558-1570; Aya and Stern “Hyaluronan in wound healing: Rediscovering a major player.” Wound Repair Regen. (2014) 22:579-593; and Frenkel “The role of hyaluronan in wound healing” Int. Wound J (2014) 11:159-163, the entire contents of each of which are incorporated herein by reference in their entirety for the purposes described herein. Accordingly, in some embodiments, HA or variants thereof that may be included in a provided polymer combination preparation can have a low molecular weight, for example, an average molecular weight of 500 kDa or less, including, e.g., 450 kDa, 400 kDa, 350 kDa, 300 kDa, 250 kDa, 200 kDa, 150 kDa, 100 kDa, 50 kDa, or less. In some embodiments, HA or variants thereof that may be included in a provided polymer combination preparation may have an average molecular weight of about 100 kDa to about 200 kDa. In some embodiments, HA or variants thereof that may be included in a provided polymer combination preparation may have an average molecular weight of about 100 kDa to about 150 kDa. In some embodiments, HA or variants thereof that may be included in a provided polymer combination preparation may have an average molecular weight of about 250 kDa to about 350 kDa. In some embodiments, HA or variants thereof that may be included in a provided polymer combination preparation may have an average molecular weight of about 300 kDa to about 400 kDa. In some embodiments, a polymer combination preparation described herein may comprise a poloxamer (e.g., ones described herein) and low molecular weight HA or variants thereof in the absence of an immunomodulatory payload may be useful for inducing innate immunity agonism.

In some embodiments, HA or variants thereof that may be included in a provided polymer combination preparation can have a high molecular weight, for example, an average molecular weight of greater than 500 kDa or higher, including, e.g., 550 kDa, 600 kDa, 650 kDa, 700 kDa, 750 kDa, 800 kDa, 850 kDa, 900 kDa, 950 kDa, 1 MDa, 1.1 MDa, 1.2 MDa, 1.3 MDa, 1.4 MDa, 1.5 MDa, 1.6 MDa, 1.7 MDa, 1.8 MDa, 1.9 MDa, 2 MDa, 2.5 MDa, 3 MDa, 3.5 MDa, 4 MDa, 4.5 MDa, or higher. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 700 kDa to about 900 kDa. In some embodiments, HA or variants thereof that may be included in a provided polymer combination preparation may have an average molecular weight of about 500 kDa to about 800 kDa. In some embodiments, HA or variants thereof that may be included in a provided polymer combination preparation may have an average molecular weight of about 600 kDa to about 800 kDa. In some embodiments, HA or variants thereof that may be included in a provided polymer combination preparation may have an average molecular weight of about 700 kDa to about 800 kDa. In some embodiments, HA or variants thereof that may be useful in accordance with the present disclosure may have an average molecular weight of about 1 MDa to about 3 MDa. In some embodiments, a polymer combination preparation described herein may comprise a poloxamer (e.g., ones described herein) and high molecular weight HA or variants thereof in the absence of an immunomodulatory payload may be useful for resolving inflammation (e.g., immunosuppressive inflammation).

In some embodiments, a provided polymer combination preparation comprises a hyaluronic acid variant. In some embodiments, a hyaluronic acid variant is water-soluble. In some embodiments, a hyaluronic acid variant may be a chemically modified hyaluronic acid, e.g., in some embodiments, hyaluronic acid is esterified. Examples of chemical modifications to hyaluronic acid include, but are not limited to, addition of thiol, haloacetate, butanediol, diglycidyl, ether, dihydrazide, aldehyde, glycan, and/or tyramine functional groups. Additional hyaluronic acid modifications and variants are known in the art. See, e.g., Highley et al., “Recent advances in hyaluronic acid hydrogels for biomedical applications” Curr Opin Biotechnol (2016) August 40:35-40; Burdick & Prestwich, “Hyaluronic acid hydrogels for biomedical applications” Advanced Materials (2011); Prestwhich, “Hyaluronic acid-based clinical biomaterials derived for cell and molecule delivery in regenerative medicine” J. Control Release (2011) October 30; 155(2): 193-199; each of which are incorporated herein by reference in their entirety for the purposes described herein.

In some embodiments, a provided polymer combination preparation comprises at least one poloxamer present at a concentration of 12.5% (w/w) or below and a second polymer component, which may be or comprise hyaluronic acid or variant thereof. In some such embodiments, HA or a variant thereof may be present in a provided polymer combination preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), or 1% (w/w) or lower. In some embodiments, HA or a variant thereof may be present in a provided polymer combination preparation at a concentration of about 0.5% (w/w) to about 5% (w/w), e.g., at a concentration of 0.5% (w/w), 0.6% (w/w), 0.7% (w/w), 0.8% (w/w), 0.9% (w/w), 1% (w/w), 1.5% (w/w), 2% (w/w), 2.5% (w/w), 3% (w/w), 3.5% (w/w), 4% (w/w), 4.5% (w/w), or 5% (w/w). In some embodiments, HA or a variant thereof having a low molecular weight (e.g., as described herein) may be present in a provided polymer combination preparation at a concentration of at least about 1.5% (w/w) or higher, including, e.g., at least 2% (w/w), at least 2.5% (w/w), at least 3% (w/w), at least 4% (w/w), at least 5% (w/w), at least 6% (w/w), at least 7% (w/w), at least 8% (w/w), at least 9% (w/w), or higher. In some embodiments, HA or a variant thereof having a low molecular weight (e.g., as described herein) may be present in a provided polymer combination preparation at a concentration of about 1.5% (w/w) to about 5% (w/w). In some embodiments, HA or a variant thereof having a low molecular weight (e.g., as described herein) may be present in a provided polymer combination preparation at a concentration of about 0.5% (w/w) to about 10% (w/w). In some embodiments, HA or a variant thereof having a low molecular weight (e.g., as described herein) may be present in a provided polymer combination preparation at a concentration of about 1% (w/w) to about 10% (w/w) or about 1.5% (w/w) to about 10% (w/w). In some embodiments, HA or a variant thereof having a low molecular weight (e.g., as described herein) may be present in a provided polymer combination preparation at a concentration of about 0.7% (w/w) to about 4% (w/w) or about 1.5% (w/w) to about 4% (w/w). In some embodiments, HA or a variant thereof having a low molecular weight (e.g., as described herein) may be present in a provided polymer combination preparation at a concentration of about 3% (w/w) to about 7% (w/w). In some embodiments, HA or a variant thereof having a high molecular weight (e.g., as described herein) may be present in a provided polymer combination preparation at a concentration of 2% (w/w) or lower, including, e.g., 1.5% (w/w), 1.25% (w/w), 1% (w/w), or lower. In some embodiments, HA or a variant thereof having a high molecular weight (e.g., as described herein) may be present in a provided polymer combination preparation at a concentration of about 0.5% (w/w) to about 3% (w/w).

### (ii) Exemplary Chitosan and Variants Thereof

In some embodiments, a carbohydrate polymer included in a provided polymer combination preparation comprising poloxamer (e.g., as described herein) may be or comprise chitosan or a variant thereof. Examples of chitosan and/or variants thereof that can be included in a polymer combination preparation described herein include, but are not limited to chitosan, chitosan salts (e.g., chitosan HCl, chitosan chloride, chitosan lactate, chitosan acetate, chitosan glutamate), alkyl chitosan, aromatic chitosan, carboxyalkyl chitosan (e.g., carboxymethyl chitosan), hydroxyalkyl chitosan (e.g., hydroxypropyl chitosan, hydroxyethyl chitosan), aminoalkyl chitosan, acylated chitosan, phosphorylated chitosan, thiolated chitosan, quaternary ammonium chitosan (e.g., N-(2-hydroxyl) propyl-3-trimethyl ammonium chitosan chloride), guanidinyl chitosan, chitosan oligosaccharide, glycated chitosan (e.g., N-dihydrogalactochitosan), chitosan poly(sulfonamides), chitosan-phenylsuccinic acid (e.g., products formed from the reaction of phenylsuccinic anhydride or a variant thereof (including, e.g., 2-phenylsuccinic anhydride, 2-phenylsuccinic acid derivatives, 2-O-acetyl L-Malic anhydride, etc.)) and chitosan (e.g., Chitosan Phenylsuccinic acid hemi-amide—ring opened amide-carboxylic acid derivative), and variants or combinations thereof. In some embodiments, a carbohydrate polymer included in a provided polymer combination preparation comprising poloxamer (e.g., as described herein) may be or comprise carboalkyl chitosan (e.g., carboxymethyl chitosan).

Those skilled in the art will appreciate that, in some cases, chitosan and/or variants thereof can be produced by deacetylation of chitin. In some embodiments, chitosan or variants thereof included in a polymer combination preparation comprising poloxamer (e.g., as described herein) is characterized by degree of deacetylation (i.e., percent of acetyl groups removed) of at least 70% or above, including, e.g., at least 75%, at least 80%, at least 85%, at least 90%, at least 95%, or higher (including up to 100%). In some embodiments, a chitosan or variants thereof is characterized by degree of deacetylation of no more than 99%, no more than 95%, no more than 90%, no more than 85%, no more than 80%, no more than 75% or lower. Combinations of the above-mentioned ranges are also possible. For example, a chitosan or variants thereof may be characterized by degree of deacetylation of 80%-95%, 70%-95%, or 75%-90%. As will be recognized by one of those skilled in the art, degree of deacetylation (% DA) can be determined by various methods known in the art, e.g., in some cases, by NMR spectroscopy.

In some embodiments, chitosan or variants thereof included in a polymer combination preparation comprising poloxamer (e.g., as described herein) may have an average molecular weight of at least 5 kDa or higher, including, e.g., at least 10 kDa or higher, including, e.g., at least 20 kDa, at least 30 kDa, at least 40 kDa, at least 50 kDa, at least 60 kDa, at least 70 kDa, at least 80 kDa, at least 90 kDa, at least 100 kDa, at least 110 kDa, at least 120 kDa, at least 130 kDa, at least 140 kDa, at least 150 kDa, at least 160 kDa, at least 170 kDa, at least 180 kDa, at least 190 kDa, at least 200 kDa, at least 210 kDa, at least 220 kDa, at least 230 kDa, at least 240 kDa, at least 250 kDa, at least 260 kDa, at least 270 kDa, at least 280 kDa, at least 290 kDa, at least 300 kDa, at least 350 kDa, at least 400 kDa, at least 500 kDa, at least 600 kDa, at least 700 kDa, or higher. In some embodiments, chitosan or variants thereof included in a polymer combination preparation comprising poloxamer (e.g., as described herein) may have an average molecular weight of no more than 750 kDa or lower, including, e.g., no more than 700 kDa, no more than 600 kDa, no more than 500 kDa, no more than 400 kDa, no more than 300 kDa, no more than 200 kDa, no more than 100 kDa, no more than 50 kDa, or lower. Combinations of the above-mentioned ranges are also possible. For example, in some embodiments, chitosan or variants thereof included in a polymer combination preparation comprising poloxamer (e.g., as described herein) is characterized by an average molecular weight of 10 kDa to 700 kDa, or 20 kDa to 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a polymer combination preparation comprising poloxamer (e.g., as described herein) is characterized by an average molecular weight of 20 kDa to 700 kDa, or 30 kDa to 500 kDa. As noted herein, an average molecular weight may be a number average molecular weight, weight average molecular weight, or peak average molecular weight.

In some embodiments, chitosan or variants thereof included in a polymer combination preparation comprising poloxamer (e.g., as described herein) is characterized by a molecular weight distribution in a range of 10 kDa to 700 kDa, or 20 kDa or 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a polymer combination preparation comprising poloxamer (e.g., as described herein) is characterized by a molecular weight distribution in a range of 20 kDa to 700 kDa, or 30 kDa to 500 kDa.

In some embodiments, chitosan or variants thereof included in a polymer combination preparation comprising poloxamer (e.g., as described herein) may be characterized by a viscosity of no more than 3,500 mPa·s or lower, including, e.g., no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, chitosan or variants thereof may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, or higher. Combinations of the above-mentioned ranges are also possible. For example, in some embodiments, such a viscous polymer solution of or comprising chitosan or variants thereof may be characterized by a viscosity of 5 mPa·s to 3,000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of chitosan or variants thereof described herein is measured at 1% in 1% acetic acid at 20° C.

In some embodiments, a polymer combination preparation comprising poloxamer (e.g., as described herein) comprises at least one or more (e.g., 1, 2, 3 or more) chitosan and/or variants thereof (including, e.g., modified chitosan and/or salts of chitosan or modified chitosan such as a chloride salt or a glutamate salt). For example, in some embodiments, chitosan and/or variants thereof (including, e.g., modified chitosan and/or salts of chitosan or modified chitosan such as a chloride salt or a glutamate salt) may be characterized by degree of deacetylation of 70%-95%, or 75%-90%, or 80%-95%, or greater than 90%. In some embodiments, chitosan and/or variants thereof (including, e.g., modified chitosan and/or salts of chitosan or modified chitosan such as a chloride salt or a glutamate salt) may be characterized by an average molecular weight of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., modified chitosan and/or salts of chitosan or modified chitosan such as a chloride salt or a glutamate salt) may be characterized by a molecular weight distribution in the range of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., salts thereof such as a chloride salt or a glutamate salt) may be characterized by a viscosity ranging from 5 to 3,000 mPa·s, or 5 to 300 mPa·s, or 20 to 200 mPa·s. In some embodiments, such chitosan and/or variants thereof (including, e.g., salts thereof such as a chloride salt or a glutamate salt) may be or comprise PROTASAN™ UltraPure chitosan chloride and/or chitosan glutamate salt (e.g., obtained from NovoMatrix®, which is a business unit of FMC Health and Nutrition (now a part of Du Pont; Product No. CL 113, CL 114, CL 213, CL 214, G 113, G 213, G 214). In some embodiments, such chitosan and/or variants thereof (including, e.g., salts thereof such as a chloride salt or a glutamate salt) may be or comprise chitosan, chitosan oligomers, and/or variants thereof (including, e.g., Chitosan HCl, carboxymethyl chitosan, chitosan lactate, chitosan acetate), e.g., obtained from Heppe Medical Chitosan GMBH (e.g., Chitoceuticals® or Chitoscience®).

In some embodiments, chitosan or variants thereof included in a polymer combination preparation comprising poloxamer (e.g., as described herein) is or comprises carboxyalkyl chitosan (e.g., carboxymethyl chitosan) that is characterized by at least one or all of the following characteristics: (1) degree of deacetylation of 80%-95%; (ii) an average molecular weight of 30 kDa to 500 kDa; or a molecular weight distribution of 30 kDa to 500 kDa; and (iii) a viscosity ranging from 5 to 300 mPa·s.

In some embodiments, chitosan or variants thereof included in a polymer combination preparation comprising poloxamer (e.g., as described herein) is or comprises a variant of chitosan (e.g., as described herein). In some embodiments, such a variant of chitosan may include chemical modification(s) of one or more chemical moieties, e.g., hydroxyl and/or amino groups, of the chitosan chains. In some embodiments, such a variant of chitosan is or comprises a modified chitosan such as, e.g., but not limited to a glycated chitosan (e.g., chitosan modified by addition of one or more monosaccharide or oligosaccharide side chains to one or more of its free amino groups). Exemplary glycated chitosan that are useful herein include, e.g., but are not limited to ones described in U.S. Pat. Nos. 5,747,475, 6,756,363, WO 2013/109732, US 2018/0312611, and US 2019/0002594, the contents of each of which are incorporated herein by reference for the purposes described herein.

In some embodiments, chitosan or variants thereof included in a polymer combination preparation comprising poloxamer (e.g., as described herein) is or comprises chitosan conjugated with a polymer that increases its solubility in aqueous environment (e.g., a hydrophilic polymer such as polyethylene glycol).

In some embodiments, chitosan or variants thereof included in a polymer combination preparation comprising poloxamer (e.g., as described herein) is or comprises thiolated chitosan. Various modifications to chitosans, e.g., but not limited to carboxylation, PEGylation, galactosylation (or other glycations), and/or thiolation are known in the art, e.g., as described in Ahmadi et al. Res Pharm Sci., 10(1): 1-16 (2015), the contents of which are incorporated herein by reference for the purposes described herein. Those skilled in the art reading the present disclosure will appreciate that other modified chitosans can be useful for a particular application in which a method is being practiced.

In some embodiments, a provided polymer combination preparation comprises at least one poloxamer present at a concentration of 12.5% or below and a second polymer component, which may be or comprise chitosan or variant thereof. In some such embodiments, chitosan or a variant thereof may be present in a provided polymer combination preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w), or lower. In some embodiments, chitosan or a variant thereof may be present in a provided polymer combination preparation at a concentration of 0.1% (w/w) to 10% (w/w), or 0.1% (w/w) to 8% (w/w), or 0.1% (w/w) to 5% (w/w), or 1% (w/w) to 5% (w/w), or about 1% (w/w) to about 3% (w/w).

### C. Payloads (e.g., Therapeutic Agents)

Provided biomaterial compositions comprise resiquimod (e.g., as a payload or therapeutic agent). In some embodiments, provided biomaterial compositions comprise resiquimod in a concentration of 0.005 mg/mL, 0.01 mg/mL, 0.02 mg/mL, 0.05 mg/mL, 0.10 mg/mL, 0.12 mg/mL, 0.14 mg/mL, 0.16 mg/mL, 0.18 mg/mL, 0.20 mg/mL, 0.22 mg/mL, 0.25 mg/mL, 0.30 mg/mL, 0.35 mg/mL, 0.40 mg/mL, 0.45 mg/mL, 0.50 mg/mL, 0.55 mg/mL, 0.60 mg/mL, 0.65 mg/mL, 0.70 mg/mL, 0.75 mg/mL, 0.80 mg/mL, 0.85 mg/mL, 0.90 mg/mL, 0.95 mg/mL, 1.00 mg/mL, 1.05 mg/mL, 1.10 mg/mL, 1.15 mg/mL, 1.20 mg/mL, 1.25 mg/mL, 1.50 mg/mL, or 1.80 mg/mL. In some embodiments, provided biomaterial compositions comprise resiquimod in a concentration of from 0.005 mg/mL to 1.80 mg/mL, from 0.01 mg/mL to 1.80 mg/mL, from 0.01 mg/mL to 0.50 mg/mL, from 0.005 mg/mL to 1.00 mg/mL, from 0.05 mg/mL to 1.00 mg/mL, from 0.05 mg/mL to 0.50 mg/mL, from 0.05 mg/mL to 0.30 mg/mL, from 0.05 mg/mL to 0.20 mg/mL, from 0.10 mg/mL to 0.25 mg/mL, from 0.10 mg/mL to 0.20 mg/mL, from 0.12 mg/mL to 0.18 mg/mL, or from 0.14 mg/mL to 0.20 mg/mL.

In some embodiments, provided biomaterial compositions comprise resiquimod as the sole immunomodulatory payload.

In some embodiments, provided biomaterial compositions may further comprise and/or be administered in combination with one or more additional payloads (e.g., one or more additional therapeutic agents, e.g., immunomodulatory agents). Exemplary immunomodulatory agents include those described in WO 2018/045058, WO 2019/183216, and PCT/US21/46392, the contents of each of which are hereby incorporated by reference.

### D. Solvent Systems

In some embodiments, a polymer combination preparation, or individual components of a polymer combination preparation, are prepared or present in a suitable solvent system. For example, in some embodiments such a solvent system has a pH ranging from 4.5-8.5. In certain embodiments, such a solvent system has a pH ranging from 4.5-7. In certain embodiments, a polymer combination preparation, or individual components of a polymer combination preparation is prepared or present in a suitable solvent system having pH 7-9. In certain embodiments, a polymer combination preparation, or individual components of a polymer combination preparation is prepared or present in a suitable solvent system having pH 7-7.5 (e.g., pH 7.4). In certain embodiments, a polymer combination preparation, or individual components of a polymer combination preparation is prepared or present in a suitable solvent system having pH 7.5-8.5. In certain embodiments, a polymer combination preparation, or individual components of a polymer combination preparation is prepared or present in a suitable solvent system having pH 8.

In certain embodiments, a polymer combination preparation, or individual components of such a polymer combination preparation are prepared or present in water. In some embodiments, a polymer combination preparation, or individual components of such a polymer combination preparation are prepared or present in an aqueous buffer system. In some embodiments, such an aqueous buffer system may comprise one or more salts (e.g., but not limited to sodium phosphate and/or sodium hydrogen carbonate). In some embodiments, such a solvent system is an aqueous buffer system having a higher buffering capacity than a 10 mM phosphate buffer. In some embodiments, such a solvent system is an aqueous buffer system having a higher buffering capacity than a 20 mM phosphate buffer. In certain embodiments, a polymer combination preparation, or individual components of such a polymer combination preparation are prepared or present in a phosphate buffer, e.g., phosphate-buffered-saline (PBS). In certain embodiments, a polymer combination preparation, or individual components of such a polymer combination preparation are prepared or present in a bicarbonate buffer. In some embodiments, polymer combination preparations, and/or individual components thereof are prepared or present in an aqueous buffer system having a concentration range of from 1 mM to 500 mM, or from 5 mM to 250 mM, or from 10 mM to 150 mM, or from 1 mM to 50 mM, or from 5 mM to 50 mM, or from 5 mM to 100 mM, or from 50 mM to 100 mM. In certain embodiments, a suitable aqueous buffer (e.g., a phosphate buffer) is prepared at a concentration of 10 mM to 50 mM. In certain embodiments, a suitable aqueous buffer (e.g., a phosphate buffer) is prepared at a concentration of 10 mM to 30 mM. In certain embodiments, a suitable aqueous buffer (e.g., a bicarbonate buffer) is prepared at a concentration of 100 mM to 200 mM. In certain embodiments, a polymer combination preparation, or individual components thereof are prepared or present in a sodium phosphate buffer at a concentration of 10 mM to 50 mM or 10 mM to 30 mM. In some embodiments, an aqueous buffer system may comprise 0.9% saline. In some embodiments, an aqueous buffer system may comprise from 0.5 wt % saline to 1.5 wt % saline, or from 0.5 wt % saline to 1.0 wt % saline.

It will be appreciated that the concentration of an aqueous buffer system may change when combined with one or more polymer components, payloads, and/or other components. Generally, when a concentration of an aqueous buffer system is specified throughout this disclosure, the concentration refers to the concentration prior to combining with one or more polymer components, payloads, and/or other components.

### E. Optional Additives

In some embodiments, a polymer combination preparation may comprise one or more additives. In some embodiments, such an additive may be or comprise a thickening agent. As will be understood by one of skilled in the art, such a thickening agent may improve suspensions of components or emulsions, which increase stability of a combination. In some embodiments, such a thickening agent may be useful to prevent, reduce, or delay phase separation of individual polymer components in a polymer combination preparation. Examples of thickening agents may include, but are not limited to cellulose derivatives, starches, pectin, xanthan, and/or any combinations thereof.

II. Certain Properties and/or Characteristics of Provided Polymer Combination Preparations or Compositions Comprising the Same

Provided polymer combination preparations or compositions comprising the same may be characterized by one or more (e.g., one, two, three, or more) of certain properties and/or characteristics described herein. Those skilled in the art, reading the present disclosure, will appreciate that provided polymer combination preparations or compositions comprising the same may be configured to provide suitable material properties and/or characteristics for a particular application. For example, in some embodiments, suitable material properties and/or characteristics for a particular application may be determined, for example, based on characteristics of tissue surrounding a tumor, administration routes, administration sites, and/or desired duration of immunomodulation in which a method is being practiced.

### A. Immunomodulatory Characteristics

In some embodiments, a provided polymer combination preparation may be non-immunomodulatory. In some such embodiments, a provided polymer combination preparation and/or a composition comprising the same may comprise an immunomodulatory payload (e.g., resiquimod) such that the resulting composition or preparation is immunomodulatory.

In some embodiments, a provided polymer combination preparation comprising poloxamer may comprise a second polymer component or an additional polymer component such that the resulting polymer combination preparation itself may be immunomodulatory in the absence of an immunomodulatory payload.

In some embodiments, a provided polymer combination preparation and/or a composition or preparation comprising a provided polymer combination preparation can, indirectly or directly, activate one or more pattern recognition receptors of one or more types of cells of an innate immune system, such as, e.g., dendritic cells, macrophages, monocytes, neutrophils, and/or natural killer (NK) cells, such that at least one or more innate immune responses are induced (e.g., as described herein). Examples of such a pattern recognition receptor is or comprises a C-type Lectin Receptor (CLR), a Nucleotide-binding Oligomerization Domain-Like Receptor (NOD-Like receptor or NLR), a Retinoic acid-inducible gene-I-Like Receptor (RLR), and/or a Toll-Like Receptor (TLR). In some embodiments, a provided polymer combination preparation and/or a composition or preparation comprising a provided polymer combination preparation can, directly or indirectly, activate at least one or more C-type Lectin Receptors (CLRs) of many different cells of an innate immune system (e.g., dendritic cells, macrophages, etc.), which include, e.g., mannose receptors, and/or asialoglycoprotein receptor family (e.g., Dectin-1, Dectin-2, macrophage-inducible C-type lectin (Mincle), dendritic cell-specific ICAM3-grabbing nonintegrin (DC-SIGN), and DC NK lectin group receptor-1 (DNGR-1)). In some embodiments, a provided polymer combination preparation and/or a composition or preparation comprising a provided polymer combination preparation can, directly or indirectly, activate at least one or more NOD-Like Receptors (NLRs) of different types of leukocytes (e.g., lymphocytes, macrophages, dendritic cells), which include, e.g., NLRA (e.g., CIITA), NLRB (e.g., NAIP), NLRC (e.g., NOD1, NOD2, NLRC3, NLRC4, NLRC5, NLRX1) and/or NLRP (e.g., NLRP1, NLRP2, NLRP3, NLRP4, NLRP5, NLRP6, NLRP7, NLRP8, NLRP9, NLRP10, NLRP11, NLRP12, NLRP13, NLRP14). In some embodiments, a provided polymer combination preparation and/or a composition or preparation comprising a provided polymer combination preparation can, directly or indirectly, activate at least one or more RIG-I-Like Receptors (RLRs) of, e.g., myeloid cells, which include, e.g., RIG-I, MDA5, and/or LGP2. In some embodiments, a provided polymer combination preparation and/or a composition or preparation comprising a provided polymer combination preparation can, directly or indirectly, activate at least one or more Toll-Like Receptors (TLRs) of different types of leukocytes (e.g., dendritic cells, myeloid dendritic cells, monocytes, macrophages, and/or neutrophils), which include, e.g., TLR1, TLR2, TLR3, TLR4, TLR5, TLR6, TLR7, TLR8, TLR9, and/or TLR10.

In some embodiments, a provided polymer combination preparation and/or a composition or preparation comprising a provided polymer combination preparation can, indirectly or directly, activate or induce (e.g., increase level and/or activity of) an inflammasome, e.g., in myeloid cells, such that at least one or more innate immune responses (and/or one or more features of an innate immune response) are induced (e.g., as described herein). In some embodiments, an inflammasome is typically a multi-protein complex that activates one or more inflammatory responses, such as, e.g., promoting maturation and/or secretion of one or more proinflammatory cytokines such as, e.g., interleukin 1p and/or interleukin 18. In some embodiments, a provided polymer combination preparation and/or a composition or preparation comprising a provided polymer combination preparation can, indirectly or directly, activate or induce (e.g., increase level and/or activity of) an inflammasome comprising an Absent in Melanoma 2 (AIM2)-Like Receptor (“AIM2 inflammasome”). In some embodiments, a provided polymer combination preparation and/or a composition or preparation comprising a provided polymer combination preparation can, indirectly or directly, activate or induce (e.g., increase level and/or activity of) an inflammasome comprising one or more NLRs, including, e.g., NLRP1 (e.g., NALP1b), NLRP3 (e.g., NALP3), and/or NLRC4 (e.g., IPAF).

In some embodiments, a provided polymer combination preparation and/or a composition or preparation comprising a provided polymer combination preparation can, directly or indirectly, activate one or more components involved in a cGAS-STING pathway (e.g., a cGAS-STING pathway and/or components thereof as described in Chen et al., “Regulation and function of the cGAS-STING pathway of cytosolic DNA sensing” Nature Immunology (2016) 17: 1142-1149); which is incorporated herein by reference in its entirety for the purpose described herein, such that innate immunity is induced. In some embodiments, a provided polymer combination preparation and/or a composition or preparation comprising a provided polymer combination preparation can, directly or indirectly, induce activity and/or level of NFκB and/or other components associated with an NFκB pathway (e.g., NFκB activation during innate immune response, e.g., as described in Dev et al., “NF-κB and innate immunity” Curr. Top. Microbiol. Immunol. (2011) 349: 115-43); which is incorporated herein by reference in its entirety for the purpose described herein. In some embodiments, a provided polymer combination preparation and/or a composition or preparation comprising a provided polymer combination preparation can, directly or indirectly, lead to production of reactive oxygen species, e.g., during innate immune response.

As will be clear to one of those skilled in the art reading the present disclosure, in some embodiments, a provided polymer combination preparation and/or a composition or preparation comprising a provided polymer combination preparation can, directly or indirectly, activate one or more of components and/or pathways (e.g., ones as described herein) associated with activation of innate immunity. For example, in some embodiments, a provided polymer combination preparation and/or a composition or preparation comprising a provided polymer combination preparation can, directly or indirectly, activate one or more pattern recognition receptors of one or more types of cells of an innate immune system (e.g., ones as described herein) and also activate or induce (e.g., increase level and/or activity of) an inflammasome, e.g., in myeloid cells.

### B. Viscosity

In some embodiments, a polymer combination preparation described herein (e.g., a precursor state or a polymer network state such as a viscous solution) can be characterized by a viscosity of no more than 25,000 mPa·s or lower, including, e.g., no more than 24,000 mPa·s, no more than 23,000 mPa·s, no more than 22,000 mPa·s, no more than 21,000 mPa·s, no more than 20,000 mPa·s, no more than 19,000 mPa·s, no more than 18,000 mPa·s, no more than 17,000 mPa·s, no more than 16,000 mPa·s, no more than 15,000 mPa·s, no more than 14,000 mPa·s, no more than 13,000 mPa·s, no more than 12,000 mPa·s, no more than 11,000 mPa·s, no more than 10,000 mPa·s, no more than 9,000 mPa·s, no more than 8,000 mPa·s, no more than 7,000 mPa·s, no more than 6,000 mPa·s, no more than 5,000 mPa·s, no more than 4,000 mPa·s, no more than 3,500 mPa·s, no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, a polymer combination preparation described herein (e.g., a precursor state or a polymer network state such as, e.g., a viscous solution) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, at least 3,000 mPa·s, at least 4,000 mPa·s, at least 5,000 mPa·s, at least 6,000 mPa·s, at least 7,000 mPa·s, at least 8,000 mPa·s, at least 9,000 mPa·s, at least 10,000 mPa·s, at least 11,000 mPa·s, at least 12,000 mPa·s, at least 13,000 mPa·s, at least 14,000 mPa·s, at least 15,000 mPa·s, at least 16,000 mPa·s, at least 17,000 mPa·s, at least 18,000 mPa·s, at least 19,000 mPa·s, at least 20,000 mPa·s, at least 21,000 mPa·s, at least 22,000 mPa·s, at least 23,000 mPa·s, at least 24,000 mPa·s, or higher. Combinations of the above-mentioned ranges are also possible. For example, in some embodiments, a polymer combination preparation described herein (e.g., a precursor state or a polymer network state such as, e.g., a viscous solution) may be characterized by a viscosity of 5 mPa·s to 10,000 mPa·s, or 10 mPa·s to 5,000 mPa·s, or 5 mPa·s to 200 mPa·s, or 20 mPa·s to 100 mPa·s, or 5 mPa·s to 20 mPa·s, or 3 mPa·s to 15 mPa·s. In some embodiments, a polymer combination preparation described herein (e.g., a precursor state or a polymer network state such as, e.g., a viscous solution) can be a viscous solution with a viscosity similar to honey (e.g., with mPa·s and/or centipoise similar to honey, e.g., approximately 2,000 to 10,000 mPa·s). In some embodiments, a polymer combination preparation described herein (e.g., a precursor state or a polymer network state such as, e.g., a viscous solution) can be a viscous solution with a viscosity similar to natural syrup (e.g., a syrup from tree sap, a syrup from molasses, etc.) (e.g., with mPa·s and/or centipoise similar to natural syrups, e.g., approximately 15,000 to 20,000 mPa·s). In some embodiments, a polymer combination preparation described herein (e.g., a precursor state or a polymer network state such as, e.g., a viscous solution) can be a viscous solution with a viscosity similar to ketchup (e.g., tomato ketchup, e.g., with mPa·s and/or centipoise similar to ketchup, e.g., approximately 5,000 to 20,000 mPa·s). One skilled in the art reading the present disclosure will appreciate that, in some cases, viscosity of a polymer combination preparation described herein may be selected or adjusted based on, e.g., administration routes (e.g., injection vs. implantation), injection volume and/or time, and/or impact duration of innate immunity stimulation. As will be also understood by one skilled in the art, viscosity of a polymer depends on, e.g., temperature and concentration of the polymer in a testing sample. In some embodiments, viscosity a polymer combination preparation described herein may be measured at 20° C., e.g., with a shear rate of 1000 s¹.

In some embodiments, a polymer combination preparation (e.g., a precursor state or a polymer network state such as, e.g., a viscous solution) comprising poloxamer (e.g., as described herein) may be characterized by a viscosity of no more than 3,500 mPa·s or lower, including, e.g., no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, polymer combination preparations (e.g., a precursor state or a polymer network state such as, e.g., a viscous solution) comprising poloxamer (e.g., as described herein) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, or higher. Combinations of the above-mentioned ranges are also possible. For example, in some embodiments, such a viscous polymer solution (e.g., a precursor state or a polymer network state such as, e.g., a viscous solution) may be characterized by a viscosity of 5 mPa·s to 3,000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of a polymer combination preparation described herein may be measured at 20° C., e.g., with a shear rate of 1000 s¹.

Among other things, the present disclosure appreciates that hydrogel technologies comprising certain crosslinking technologies (e.g., certain chemical crosslinking technologies, ultraviolet light, etc.) may produce toxic by-products and/or may adversely affect stability or efficacy of agents (e.g., therapeutic agents) that may be combined with a polymer combination preparation.

Alternatively or additionally, the present disclosure appreciates that, in some embodiments, particular advantages can be achieved by administering component(s) of a polymer combination preparation so that an immunomodulatory composition as described herein is formed during and/or upon administration as compared with pre-forming (e.g., by cross-linking) a polymer biomaterial prior to introducing it into a subject. For example, administration of a preformed biomaterial requires proportionate incisions and/or surgical interventions to facilitate administration. In some embodiments, for example, the present disclosure appreciates that such pre-forming generates a material with a defined size and/or structure, which may restrict options for administration, as the dimensions of the pre-formed material may differ from those of a target site (e.g., a resection cavity). In some embodiments a hydrogel may be formed during and/or upon administration. In some embodiments, a polymer combination preparation administered to a target site may comprise a pre-formed hydrogel polymer combination preparation.

In some embodiments, the present disclosure appreciates that a polymer combination preparation that is useful for administration to a target site described herein may be a viscous liquid solution. For example, in some embodiments, a liquid polymer combination preparation may be introduced to a target site so that an immunomodulatory composition as described herein in a form of a viscous solution (e.g., a solution with a viscosity of about 5,000 to 15,000 centipoise at body temperature, e.g., a solution with a viscosity of about 10,000 centipoise at body temperature) is formed upon administration to a target site.

In some embodiments, the present disclosure appreciates that a polymer combination preparation that is useful for administration to a target site described herein may be a viscous liquid solution, which can be substantially retained at the target site upon administration for a certain period of time. In some embodiments, such a viscous liquid polymer combination preparation has a viscosity that is low enough to be injectable (e.g., through a syringe tip or a catheter and/or a syringe needle) but is high enough to be substantially retained at a target site upon administration for a certain period of time. In some embodiments, such a viscous liquid polymer combination preparation may have a viscosity of about 500 to 10,000 centipoise at room temperature. In some embodiments, such a viscous liquid polymer combination preparation may have a viscosity of about 500 to 3,000 centipoise at room temperature. In some embodiments, such a viscous liquid polymer combination preparation may have a viscosity of about 1,000 to 8,000 centipoise at room temperature. In some embodiments, such a viscous liquid polymer combination preparation may have a viscosity of about 2,000 to 6,000 centipoise at room temperature. In some embodiments, such a viscous liquid polymer combination preparation may have a viscosity of about 3,000 to 7,000 centipoise at room temperature. In some embodiments, such a viscous liquid polymer combination preparation may have a viscosity of about 4,000 to 8,000 centipoise at room temperature. In some embodiments, such a viscous liquid polymer combination preparation may have a viscosity of about 5,000 to 9,000 centipoise at room temperature. In some embodiments, such a viscous liquid polymer combination preparation may have a viscosity of about 6,000 to 10,000 centipoise at room temperature.

In some embodiments, the present disclosure appreciates that there may be a viscosity constraint and/or limit on injectability of a liquid polymer combination preparation. For example, in some embodiments, an injectable polymer combination preparation may be characterized by a viscosity amenable to loading and controlled release through a needle of a set gauge (e.g., a needle with a gauge of between 14 and 20, e.g., a needle with a gauge of 16-18). Alternatively, in some embodiments, an injectable polymer combination preparation may be characterized by a viscosity amenable to loading and controlled release through a syringe tip of a set diameter (i.e., without a connected needle, or with a catheter). In some embodiments, a polymer combination preparation included in an immunomodulatory composition (e.g., as described herein) loaded into a syringe may further comprise a plasticizer.

The present disclosure provides technologies, including particular polymer combination preparations, and methods of administration, that permit interventions that may be less invasive than implantation and/or less toxic than systemic administration. In some such embodiments, preparations with improved administration characteristics may be administered in a liquid state; in some embodiments they may be administered in a pre-formed gel state characterized by flexible space-filling properties; in some embodiments they may be administered subcutaneously; in some embodiments they may function as a proximal depot for sustained release of immunomodulatory payloads (e.g., resiquimod); in some embodiments they may permit reprogramming of tissues (e.g., such as tumors and/or e.g., such as sentinel and/or draining lymph nodes); in some embodiments they may be administered prior to or contemporaneously with a tumor resection surgery; in some embodiments, they may be administered ipsilaterally when compared to a tumor resection site and/or primary tumor site; in some embodiments, they may be administered contralaterally when compared to a tumor resection site and/or primary tumor site; in some embodiments, they may be administered to patients who have metastatic, disseminated, and/or recurrent cancers. In some such embodiments, provided preparations are comprised of a relevant material in particulate form (e.g., so that the preparations comprise a plurality of particles, e.g., characterized by a size distribution and/or other parameters as described herein).

### C. Storage Modulus: Polymer Network State

In some embodiments, when a polymer combination preparation described herein is in a polymer network state, such a polymer network state may be characterized by a storage modulus of at least 100 Pa, at least 200 Pa, at least 300 Pa, at least 400 Pa, at least 500 Pa, at least 600 Pa, at least 700 Pa, at least 800 Pa, at least 900 Pa, at least 1,000 Pa, at least 1,100 Pa, at least 1,200 Pa, at least 1,300 Pa, at least 1,400 Pa, at least 1,500 Pa, at least 1,600 Pa, at least 1,700 Pa, at least 1,800 Pa, at least 1,900 Pa, at least 2,000 Pa, at least 2,100 Pa, at least 2,200 Pa, at least 2,300 Pa, at least 2,400 Pa, at least 2,500 Pa, at least 2,600 Pa, at least 2,700 Pa, at least 2,800 Pa, at least 2,900 Pa, at least 3,000 Pa, at least 3,500 Pa, at least 4,000 Pa, at least 4,500 Pa, at least 5,000 Pa, at least 6,000 Pa, at least 7,000 Pa, at least 8,000 Pa, at least 9,000 Pa, at least 10,000 Pa, at least 11,000 Pa, at least 12,000 Pa, at least 13,000 Pa, at least 14,000 Pa, at least 15,000 Pa, or higher. In some embodiments, such a polymer network state of a provided polymer combination preparation may be characterized by a storage modulus of no more than 15 kPa, no more than 14 kPa, no more than 13 kPa, no more than 12 kPa, no more than 11 kPa, no more than 10 kPa, no more than 9 kPa, no more than 8 kPa, no more than 7 kPa, no more than 6 kPa, or lower. Combinations of the above-mentioned ranges are also possible. For example, in some embodiments, such a polymer network state of a provided polymer combination preparation may be characterized by a storage modulus of 100 Pa to 15 kPa, or 100 Pa to 10 kPa, or 100 Pa to 7.5 kPa, or 200 Pa to 5,000 Pa, or 300 Pa to 2,500 Pa, or 500 Pa to 2,500 Pa, or 100 Pa to 500 Pa. In some embodiments, a polymer network state of a provided polymer combination preparation may be characterized by a storage modulus of 1,000 Pa to 10,000 Pa, or 2,000 Pa to 10,000 Pa, or 3,000 Pa to 10,000 Pa, or 4,000 Pa to 10,000 Pa, or 5,000 Pa to 10,000, or 6,000 Pa to 10,000 Pa. One of those skilled in the art will appreciate that various rheological characterization methods (e.g., as described in Weng et al., “Rheological Characterization of in situ Crosslinkable Hydrogels Formulated from Oxidized Dextran and N-Carboxyethyl Chitosan” Biomacromolecules, 8: 1109-1115 (2007)) can be used to measure storage modulus of a material, and that, in some cases, storage modulus of a material may be measured with a rheometer and/or dynamic mechanical analysis (DMA). One of those skilled in the art will also appreciate that rheological characterization can vary with surrounding condition, e.g., temperature and/or pH. Accordingly, in some embodiments, a provided polymer combination preparation is characterized by a storage modulus (e.g., as described herein) measured at a body temperature of a subject (e.g., 37° C. of a human subject), e.g., at a pH 5-8 or at a physiological pH (e.g., pH 7). As will be clear to one skilled in the art reading the disclosure provided herein, a storage modulus of a provided polymer combination preparation, e.g., in a form of particles, refers to a bulk storage modulus of particles in a population.

In some embodiments, a polymer network state of a polymer combination preparation provided herein may be characterized by a storage modulus lower than that of an 18 wt % poloxamer hydrogel. For example, in some embodiments, a polymer network state of a polymer combination preparation provided herein may be characterized by a storage modulus, as measured at 37° C., that is reduced by at least 10% or more, including, e.g., at least 20%, at least 30%, at least 40%, at least 50%, at least 60%, or more, as compared to that of an 18% (w/w) poloxamer hydrogel.

In some embodiments, a polymer network state of a polymer combination preparation provided herein may be characterized by a storage modulus (e.g., as described herein) that maintains substantially the same (e.g., within 20% or within 10% or within 5%) when stored at an appropriate temperature for a period of time. For example, in some embodiments, a polymer network state of a polymer combination preparation provided herein may be characterized by a storage modulus (e.g., as described herein), as measured at 37° C., that maintains substantially the same (e.g., within 20% or within 10% or within 5%) when stored at a temperature of 4° C.-10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer network state of a polymer combination preparation provided herein may be characterized by a storage modulus (e.g., as described herein), as measured at 37° C., that maintains substantially the same (e.g., within 20% or within 10% or within 5%) when stored at a room temperature (e.g., 20° C.-25° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

### D. Phase Angle: Polymer Network State

In some embodiments, a polymer network state of a provided polymer combination preparation can be characterized by a phase angle indicative of a viscoelastic material. For example, in some embodiments, a polymer network state of a provided polymer combination preparation can be characterized by a phase angle of 10 to 50°, or 2° to 45°, or 3° to 40°, or 3° to 35° or 3° to 30°, or 3° to 25°, or 5° to 30°, or 10° to 30°, or 15° to 25°, or 20° to 35°. In some embodiments, a polymer network state of a provided polymer combination preparation can be characterized by a phase angle of 100 to 300 or 15° to 25°. In some embodiments, a polymer network state of a provided polymer combination preparation can be characterized by a phase angle of 5° to 150 or 10° to 20°. As will be understood by one skilled in the art, phase angle of a polymer biomaterial may be determined by dynamical mechanical analysis, e.g., a frequency sweep analysis, which includes, e.g., determination of shear storage modulus and shear loss modulus of a sample. One skilled in the art will appreciate that a storage or elastic modulus of a material may be determined based on its stored energy and it represents the elastic property of the material, while a loss or viscous modulus may be determined based on the energy dissipated as heat and it represents the viscous property of the material. The phase angle (delta) is the arctangent of the ratio of a storage modulus to a loss modulus and its value indicates if the material is more elastic or viscous. Typically, a phase angle of >450 indicates that the viscous property dominates and the material behaves more like a solution. As the phase angle approaches 0°, the elastic (solid or gel-like) property dominates. For example, a material with a high storage modulus and a low phase angle indicates a stronger gel (more elastic) than one with a lower storage modulus and phase angle. In some embodiments, the phase angle of a provided polymer combination preparation (e.g., as described herein) in a polymer network state may be determined from a frequency sweep analysis performed at a temperature corresponding to the body of the body temperature of a subject to be treated. In some embodiments, a frequency sweep analysis may be performed over a frequency range of 0.1 to 10 Hz with application of a constant 0.4% strain.

### E. Dissolution/Degradation Rate

Polymer combination preparations described herein are typically biocompatible. In some embodiments, at least one polymer component in provided polymer combination preparations may be biodegradable in vivo. In some embodiments, at least one polymer component in provided polymer combination preparations may be resistant to biodegradation (e.g., via enzymatic and/or oxidative mechanisms). In some embodiments, at least one polymer component in provided polymer combination preparations may be chemically oxidized. Accordingly, in some embodiments, polymer combination preparations are able to be degraded, chemically and/or biologically, within a physiological environment, such as within a subject's body, e.g., at a target site of a subject. One of those skilled in the art will appreciate, reading the present disclosure, that degradation rates of provided polymer combination preparations may vary, e.g., based on selection of a poloxamer type and/or a second polymer (e.g., a carbohydrate polymer such as hyaluronic acid and/or chitosan as described herein in some embodiments) and their material properties, and/or concentrations thereof (e.g., as described herein). For example, the half-life of provided polymer combination preparations (the time at which 50% of a polymer combination preparation is degraded into monomers and/or other non-polymeric moieties) may be on the order of days, weeks, months, or years. In some embodiments, polymer combination preparations described herein may be biologically degraded, e.g., by enzymatic activity or cellular machinery, for example, through exposure to a lysozyme (e.g., having relatively low pH), or by simple hydrolysis. In some cases, provided polymer combination preparations may be broken down into monomers (e.g., polymer monomers) and/or non-polymeric moieties that are non-toxic to cells. As will be understood by one of those skilled in the art, a provided polymer combination preparation has a longer residence time at a target site (e.g., a tumor resection site) upon administration if such a provided polymer combination preparation has a slower in vivo degradation rate.

In some embodiments, a polymer combination preparation provided herein remains substantially homogenous (e.g., no detectable phase separation) when stored at a temperature of 4° C.-10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer combination preparation provided herein remains substantially homogenous (e.g., no detectable phase separation) when stored at a room temperature for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

In some embodiments, a polymer combination preparation provided herein may be characterized in that no more than 20% or less, including, e.g., no more than 15%, no more than 10%, no more than 8%, no more than 6%, no more than 5%, no more than 4%, no more than 3%, no more than 2%, no more than 1%, or less, of the polymer combination preparation is degraded (e.g., via biodegradation or chemical degradation) when stored at a temperature of 4° C.-10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer combination preparation provided herein may be characterized in that no more than 20% or less, including, e.g., no more than 15%, no more than 10%, no more than 8%, no more than 6%, no more than 5%, no more than 4%, no more than 3%, no more than 2%, no more than 1%, or less, of the polymer combination preparation is degraded (e.g., via biodegradation or chemical degradation) when stored at a room temperature for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 2 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 2 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 2 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 3 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 3 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 3 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 5 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 5 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 5 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 7 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 7 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 7 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 14 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 14 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 14 days or more after the administration.

In some embodiments, a provided polymer combination preparation is characterized in that, when assessed in vivo by administering to a target site (e.g., a tumor resection site) in a test subject (e.g., as described herein), no more than 10% or less, including, e.g., no more than 9%, no more than 8%, no more than 7%, no more than 6%, no more than 5%, no more than 4%, no more than 3%, no more than 2%, no more than 1% or less, of such a provided polymer combination preparation remains at the target site in vivo 10 days or more after the administration.

In some embodiments where a provided polymer combination preparation is immunomodulatory (e.g., acting as a polymeric biomaterial agonist of innate immunity as described in PCT/US20/31169 filed May 1, 2020 (published as WO2020/223698A1)), a provided polymer combination preparation is characterized in that, when assessed in vivo by administering to a target site (e.g., a tumor resection site) in a test subject (e.g., as described herein), such a provided polymer combination preparation is dissolved or degraded at a rate such that an immune response is modulated in one or more aspects. For example, in some embodiments, such a provided polymer combination preparation is dissolved or degraded at a rate such that innate immunity is stimulated in one or more aspects (e.g., activation of a pattern recognition receptor, an inflammasome, and/or a cGAS-STING pathway; and/or production of proinflammatory cytokines and/or upregulation of antigen presentation machinery and/or costimulatory molecules) for a period of at least 2 days or more, including, e.g., at least 3 days, at least 4 days, at least 5 days, at least 6 days, at least 7 days, at least 9 days, at least 10 days or more. In some embodiments, such a provided polymer combination preparation is dissolved or degraded at a rate such that innate immunity is stimulated in one or more aspects (e.g., ones as described herein, including, e.g., but not limited to activation of a pattern recognition receptor, an inflammasome, and/or a cGAS-STING pathway; and/or production of proinflammatory cytokines and/or upregulation of antigen presentation machinery and/or costimulatory molecules) for a period of no more than 15 days or fewer, including, e.g., no more than 10 days, no more than 9 days, no more than 8 days, no more than 7 days, no more than 6 days, no more than 5 days, no more than 4 days, no more than 3 days or fewer.

### F. Payload Release Rate

In some embodiments, polymer combination preparations described herein may be useful to deliver one or more payloads (e.g., resiquimod). For example, in some embodiments, one or more payloads may be distributed in a polymer combination preparation such that when administered at a target site (e.g., at a tumor resection site), the polymer combination preparation extends the release of the therapeutic agent at the target site relative to administration of the same therapeutic agent in solution. In certain embodiments, such a polymer combination preparation can extend the release of the therapeutic agent at a target site (e.g., at a tumor resection site) relative to administration of the same therapeutic agent in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks. In some embodiments, such a polymer combination preparation can extend the release of a therapeutic agent so that, when assessed at a specified time point after administration, more therapeutic agent is present at a target administration site (e.g., a tumor resection site) than that is observed when the therapeutic agent is administered in solution. For example, in some embodiments, when assessed at 24 hours after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 48 hours after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 3 days after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 5 days after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution.

### G. In Vivo Efficacy

In some embodiments, at least one therapeutic agent (e.g., resiquimod) may be incorporated in a polymer combination preparation and/or composition comprising the same described herein. In some embodiments, such a polymer combination preparation is characterized in that a test animal group with spontaneous metastases having, at a tumor resection site, the polymer combination preparation in a polymer network state has a higher percent survival than that of a comparable test animal group having, at a tumor resection site, the same polymer combination preparation without the immunomodulatory payload, as assessed at 2 months after the administration. In some such embodiments, an increase in percent survival as observed in a test animal group with spontaneous metastases having, at a tumor resection site, a provided polymer combination preparation (incorporating an immunomodulatory payload) is at least 30% or more, including, at least 40%, at least 50%, at least 60%, at least 70%, at least 80%, at least 90%, or more, as compared to that of a comparable test animal group having, at a tumor resection site, the same polymer combination preparation without the immunomodulatory payload, as assessed at 2 months after the administration.

### III. Exemplary Embodiments of Provided Polymer Combination Preparations

In some embodiments, polymer combination preparations described herein are prepared in a phosphate buffer or a carbonate buffer at pH 7-8. In some embodiments, a phosphate buffer may have a concentration of 10-50 mM (including, e.g., 10 mM, 20 mM, 30 mM, 40 mM, or 50 mM). In some embodiments, a bicarbonate buffer may have a concentration of 25-200 mM (including, e.g., 25 mM, 50 mM, 75 mM, 100 mM, 125 mM, 150 mM, 175 mM, or 200 mM).

In some embodiments, polymer combination preparations described herein are temperature-responsive, and have a critical gelation temperature of about 10-30° C. In some embodiments, such polymer combination preparations described herein may have a critical gelation temperature of around room temperature, e.g., 10-15° C. In some embodiments, such polymer combination preparations described herein may have a critical gelation temperature of around room temperature, e.g., 15-20° C. In some embodiments, such polymer combination preparations described herein may have a critical gelation temperature of around room temperature, e.g., 20-25° C. In some embodiments, such polymer combination preparations described herein may have a critical gelation temperature of about 25-28° C. In some embodiments, such polymer combination preparations described herein may have a critical gelation temperature of about 28-32° C. In some embodiments, such polymer combination preparations described herein may have a critical gelation temperature of about 32-34° C. In some embodiments, such polymer combination preparations described herein may have a critical gelation temperature of about 34-37° C.

In certain embodiments, a polymer combination preparation comprises 5-12.5% (w/w) or 6-10% (w/w) Poloxamer 407 and 0.5-3% (w/w) hyaluronic acid having an average molecular weight of 1-2 MDa. In some embodiments, such a polymer combination preparation (e.g., upon transition to a polymer network state) may be characterized by a storage modulus, which may range from approximately 300 Pa to approximately 4,600 Pa or approximately 300 Pa to approximately 6,500 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-4,200 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, or approximately 5,800-6,500 Pa). In some embodiments, such a polymer combination preparation (e.g., upon transition to a polymer network state) may be characterized by a phase angle of about 2-20°.

In certain embodiments, a polymer combination preparation comprises 5-12.5% (w/w), 7-12.5% (w/w), 7-11.5% (w/w), 6-11.5% (w/w), 5-11.5% (w/w), 5-11% (w/w), 5-10.5% (w/w), 6-10.5% (w/w), 6-10% (w/w), 7-11% (w/w), or 8-11% (w/w) Poloxamer 407, with 0.5-3% (w/w) hyaluronic acid, 0.5-2% (w/w) hyaluronic acid, 1-2% (w/w) hyaluronic acid, 1-3% (w/w) hyaluronic acid, 1-4% (w/w) hyaluronic acid, 2-5% (w/w) hyaluronic acid, 3-6% (w/w) hyaluronic acid, or 4-7% (w/w) hyaluronic acid having an average molecular weight of 500 kDa-900 kDa. In some embodiments, such a polymer combination preparation (e.g., upon transition to a polymer network state) may be characterized by a storage modulus, which may range from approximately 100 Pa to approximately 7,600 Pa, 100 Pa to approximately 15,000 Pa, or 500 Pa to approximately 18,000 Pa. In some embodiments, such a polymer combination preparation (e.g., upon transition to a polymer network state) may be characterized by a storage modulus, which may range from approximately 300 Pa to approximately 8,000 Pa, (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-4,200 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,600 Pa, approximately 6,400-6,800 Pa, approximately 6,600-7,000 Pa, approximately 6,800-7,200 Pa, approximately 7,000-7,400 Pa, approximately 7,200-7,600 Pa, approximately 7,400-7,800 Pa, approximately 7,600-8,000 Pa). In some embodiments, such a polymer combination preparation (e.g., upon transition to a polymer network state) may be characterized by a phase angle of about 2-20°.

In certain embodiments, a polymer combination preparation including a high MW hyaluronic acid comprises a formulation described in Example 5, Table 10.

In certain embodiments, a polymer combination preparation comprises 5-12.5% (w/w), 8-12.5% (w/w), 6-11.5% (w/w), 6-11% (w/w), 7-11% (w/w), 8-11% (w/w), 6-10.5% (w/w), or 6-10% (w/w) Poloxamer 407, with 1-4% (w/w) hyaluronic acid, 2-5% (w/w) hyaluronic acid, or 1-10% (w/w) hyaluronic acid, or 1.5-10% (w/w) hyaluronic acid, or 3-6% (w/w) hyaluronic acid, or 4-7% (w/w) hyaluronic acid having an average molecular weight of 100 kDa-500 kDa. In certain embodiments, a polymer combination preparation comprises 10.9% (w/w), 10.8% (w/w), 10.7% (w/w), 10.6% (w/w), 10.5% (w/w), 10.4% (w/w), 10.3% (w/w), 10.2% (w/w), 10.1% (w/w), 10.0% (w/w), 9.9% (w/w), 9.8% (w/w), 9.7% (w/w), 9.6% (w/w), 9.5% (w/w), or 9.0% (w/w) Poloxamer 407, with 1-4% (w/w) hyaluronic acid, or 2-5% (w/w) hyaluronic acid, or 1-10% (w/w) hyaluronic acid, or 1.5-10% (w/w) hyaluronic acid, or 3-6% (w/w) hyaluronic acid, or 4-7% (w/w) hyaluronic acid having an average molecular weight of 100 kDa-500 kDa. In certain embodiments, a polymer combination preparation comprises 5-12.5% (w/w), 8-12.5% (w/w), 8-11% (w/w), 6-11% (w/w), 6-10.5% (w/w), or 6-10% (w/w) Poloxamer 407, and 0.5-10% (w/w) hyaluronic acid, or 1.5-10% (w/w) hyaluronic acid, or 2-6% (w/w) hyaluronic acid or 4-9% (w/w) hyaluronic acid having an average molecular weight of 100 kDa-300 kDa. In certain embodiments, a polymer combination preparation comprises 10.9% (w/w), 10.8% (w/w), 10.7% (w/w), 10.6% (w/w), 10.5% (w/w), 10.4% (w/w), 10.3% (w/w), 10.2% (w/w), 10.1% (w/w), 10.0% (w/w), 9.9% (w/w), 9.8% (w/w), 9.7% (w/w), 9.6% (w/w), 9.5% (w/w), or 9.0% (w/w) Poloxamer 407, and 0.5-10% (w/w) hyaluronic acid, or 1.5-10% (w/w) hyaluronic acid, or 2-6% (w/w) hyaluronic acid, or 4-9% (w/w) hyaluronic acid having an average molecular weight of 100 kDa-300 kDa. In certain embodiments, a polymer combination preparation comprises 8-12.5% (w/w) or 8-11% (w/w) Poloxamer 407 and 2-6% (w/w) hyaluronic acid having an average molecular weight of 100 kDa-200 kDa. In some embodiments, such a polymer combination preparation (e.g., upon transition to a polymer network state) may be characterized by a storage modulus, which may range from approximately 400 Pa to approximately 3,400 Pa. In some embodiments, such a polymer combination preparation (e.g., upon transition to a polymer network state) may be characterized by a phase angle of about 2-35° or 2-20°. In certain embodiments, a polymer combination preparation comprises 5-11% (w/w), 6-10.5% (w/w), or 6-10% (w/w) Poloxamer 407 and 1-10% (w/w) or 1.5-10% (w/w) hyaluronic acid having an average molecular weight of 100 kDa-200 kDa. In some embodiments, such a polymer combination preparation (e.g., upon transition to a polymer network state) may be characterized by a storage modulus, which may range from approximately 400 Pa to approximately 5,000 Pa or 300 Pa to approximately 6,500 Pa, (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-4,200 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,500 Pa). In some embodiments, such a polymer combination preparation (e.g., upon transition to a polymer network state) may be characterized by a phase angle of about 20-35°.

In certain embodiments, a polymer combination preparation including a low MW hyaluronic acid comprises a formulation described in Example 5, Table 9.

In certain embodiments, a polymer combination preparation comprises 8-12.5% (w/w) or 8-11% (w/w) Poloxamer 407 or 6-10% (w/w) Poloxamer 407, and 1-10% (w/w), or 1.5-9% (w/w) or 1-5% (w/w) or 5-10% (w/w) hyaluronic acid having an average molecular weight of 70 kDa-200 kDa or 80 kDa-150 kDa. In some embodiments, such a polymer combination preparation (e.g., upon transition to a polymer network state) may be characterized by a storage modulus, which may range from approximately 200 Pa to approximately 6,500 Pa, or approximately 200 Pa to approximately 5,900 Pa. In some embodiments, such a polymer combination preparation (e.g., upon transition to a polymer network state) may be characterized by a phase angle of about 2-35° or 2-20°. In some embodiments, such a polymer combination preparation (e.g., upon transition to a polymer network state) may be characterized by a storage modulus, which may range from approximately 400 Pa to approximately 6,500 Pa, or 400 Pa to approximately 4,600 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-4,200 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,500 Pa). In some embodiments, such a polymer combination preparation (e.g., upon transition to a polymer network state) may be characterized by a phase angle of about 2-32°, or about 15 to 35°.

In certain embodiments, a polymer combination preparation comprises 8-12.5% (w/w) or 8-11% (w/w) Poloxamer 338 and 1-3% (w/w) hyaluronic acid having an average molecular weight of 1-2 MDa. In some embodiments, such a polymer combination preparation (e.g., upon transition to a polymer network state) may be characterized by a storage modulus, which may range from approximately 980 Pa to approximately 1,300 Pa. In some embodiments, such a polymer combination preparation (e.g., upon transition to a polymer network state) may be characterized by a phase angle of about 2-35° or 2-20°.

In certain embodiments, a polymer combination preparation comprises 5-12.5% (w/w), or 8-11.5% (w/w), or 8-11% (w/w) Poloxamer 338, with 1-4% (w/w) hyaluronic acid having an average molecular weight of 500 kDa-900 kDa. In some embodiments, such a polymer combination preparation (e.g., upon transition to a polymer network state) may be characterized by a storage modulus, which may range from approximately 1,400 Pa to approximately 2,700 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 338 and 1-5% (w/w) hyaluronic acid having an average molecular weight of 100 kDa-350 kDa. In some embodiments, such a polymer combination preparation (e.g., upon transition to a polymer network state) may be characterized by a storage modulus, which may range from approximately 500 Pa to approximately 1,350 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407 and 2.5-5% (w/w) modified chitosan (e.g., carboxymethyl chitosan). In some embodiments, such a polymer combination preparation (e.g., upon transition to a polymer network state) may be characterized by a storage modulus, which may range from approximately 1,000 Pa to approximately 5,000 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 0.5-5% (w/w) hyaluronic acid having an average molecular weight of 500 kDa-900 kDa, and 0.1-1.5% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) or 6-11% (w/w) or 6-10.5% (w/w) or 6-10% (w/w) Poloxamer 407, 0.5%-10% (w/w) or 1-10% (w/w) or 1-5% (w/w) hyaluronic acid having an average molecular weight of 80 kDa-150 kDa, and 0.1-5% (w/w) or 0.2-5% (w/w) or 0.1-3% (w/w) modified chitosan (e.g., carboxymethyl chitosan and/or chitosan-phenyl succinic acid). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 1-5% (w/w) hyaluronic acid having an average molecular weight of 500 kDa-900 kDa, and 0.2-4% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 1-5% (w/w) hyaluronic acid having an average molecular weight of 100 kDa-500 kDa, and 0.2-4% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 3.5-5.5% (w/w) or 4-5% (w/w) Poloxamer 407, and 1.5-3.5% (w/w) high molecular weight hyaluronic acid (e.g., hyaluronic acid having an average molecular weight of greater than 500 kDa, such as, e.g., 600-1500 kDa, or 700-1500 kDa). In some embodiments, such a polymer combination preparation (e.g., upon transition to a polymer network state) may be characterized by a storage modulus, which may range from approximately 200 Pa to approximately 10,000 Pa, 500 Pa to approximately 9,000 Pa, or approximately 1,000 Pa to approximately 8,000 Pa, or 1,000 Pa to approximately 6,000 Pa.

In some embodiments, such polymer combination preparations further comprise resiquimod. In some embodiments, such polymer combination preparations further comprise resiquimod in a concentration of 0.02 mg/mL, 0.05 mg/mL, 0.10 mg/mL, 0.12 mg/mL, 0.14 mg/mL, 0.16 mg/mL, 0.18 mg/mL, 0.20 mg/mL, 0.22 mg/mL, 0.25 mg/mL, 0.30 mg/mL, 0.35 mg/mL, 0.40 mg/mL, 0.45 mg/mL, 0.50 mg/mL, 0.55 mg/mL, 0.60 mg/mL, 0.65 mg/mL, 0.70 mg/mL, 0.75 mg/mL, 0.80 mg/mL, 0.85 mg/mL, 0.90 mg/mL, 0.95 mg/mL, 1.00 mg/mL, 1.05 mg/mL, 1.10 mg/mL, 1.15 mg/mL, 1.20 mg/mL, 1.25 mg/mL, 1.50 mg/mL, or 1.80 mg/mL. In some embodiments, provided biomaterial compositions comprise resiquimod in a concentration of from 0.01 mg/mL to 1.80 mg/mL, from 0.01 mg/mL to 0.50 mg/mL, from 0.05 mg/mL to 1.00 mg/mL, from 0.05 mg/mL to 0.50 mg/mL, from 0.05 mg/mL to 0.30 mg/mL, from 0.05 mg/mL to 0.20 mg/mL, from 0.10 mg/mL to 0.25 mg/mL, from 0.10 mg/mL to 0.20 mg/mL, from 0.12 mg/mL to 0.18 mg/mL, or from 0.14 mg/mL to 0.20 mg/mL. In some embodiments, such polymer combination preparations comprise resiquimod as the sole immunomodulatory payload in a concentration of from 0.01 mg/mL to 0.50 mg/mL, from 0.05 mg/mL to 0.50 mg/mL, from 0.05 mg/mL to 0.30 mg/mL, from 0.05 mg/mL to 0.20 mg/mL, from 0.10 mg/mL to 0.25 mg/mL, from 0.10 mg/mL to 0.20 mg/mL, from 0.12 mg/mL to 0.18 mg/mL, or from 0.14 mg/mL to 0.20 mg/mL. In some embodiments, such polymer combination preparations further comprise resiquimod and an additional payload (e.g., an additional immunomodulatory payload).

### Methods of Preparing Provided Biomaterial Compositions

In some embodiments, the present disclosure provides methods of preparing provided biomaterial compositions (e.g., polymer combination preparations and compositions thereof). In some embodiments, provided methods of preparing provided biomaterial compositions utilize resiquimod solid forms and compositions thereof described herein. In some embodiments, resiquimod is provided and/or utilized in accordance with the present disclosure in a form such as a solid form. In some embodiments, resiquimod is provided and/or utilized in accordance with the present disclosure in an amorphous form, in a crystalline form, or in a mixture thereof. In some embodiments, resiquimod is provided and/or utilized in accordance with the present disclosure as a composition comprising one or more resiquimod solid forms described herein.

In some embodiments, the present disclosure provides a method comprising (i) providing at least one solid form of resiquimod, e.g., Resiquimod Form I, Resiquimod Form II, Resiquimod Form III, Resiquimod Form IV, Resiquimod Form V, Resiquimod Form VI, or Resiquimod Form VII; and (ii) combining the at least one solid form of resiquimod with poloxamer and a second polymer component (e.g., hyaluronic acid and/or chitosan) in an appropriate buffer.

In some embodiments, the present disclosure provides a method comprising (i) providing at least one solid form of resiquimod, e.g., Resiquimod Form I, Resiquimod Form II, Resiquimod Form III, Resiquimod Form IV, Resiquimod Form V, Resiquimod Form VI, or Resiquimod Form VII; (ii) combining the at least one solid form of resiquimod with poloxamer in an appropriate buffer; and (iii) adding a second polymer component (e.g., hyaluronic acid and/or chitosan).

In some embodiments, the present disclosure provides a method comprising (i) providing a polymer combination preparation, e.g., as described herein; and (ii) combining the polymer combination preparation with at least one solid form of resiquimod, e.g., Resiquimod Form I, Resiquimod Form II, Resiquimod Form III, Resiquimod Form IV, Resiquimod Form V, Resiquimod Form VI, or Resiquimod Form VII.

In some embodiments, the present disclosure provides a method comprising (i) mixing an appropriate amount of poloxamer and at least a second polymer component (e.g., hyaluronic acid and/or chitosan) in an appropriate buffer to provide a polymer combination preparation, e.g., as described herein; and (ii) combining the polymer combination preparation with at least one solid form of resiquimod, e.g., Resiquimod Form I, Resiquimod Form II, Resiquimod Form III, Resiquimod Form IV, Resiquimod Form V, Resiquimod Form VI, or Resiquimod Form VII.

In some embodiments, polymer combination preparations described herein may be prepared by mixing an appropriate amount of poloxamer and at least a second polymer component (e.g., hyaluronic acid and/or chitosan) in an appropriate buffer. Poloxamer and at least a second polymer component (e.g., hyaluronic acid and/or chitosan) may be independently a solid particle preparation or a liquid preparation. In some embodiments, a payload, e.g., resiquimod and, optionally an additional payload, may be added to such a polymer mixture solution. In some embodiments, a polymer mixture solution may be mixed at a low speed (e.g., a speed of less than 100 rpm) until a homogenous polymer solution is formed. To induce gel formation, such a homogenous polymer solution can be exposed to a critical gelation temperature or above for a period of time sufficient for gel formation (e.g., 10-15 mins).

In some embodiments, the present disclosure, among other things, appreciates that mixing a solid particle preparation of hyaluronic acid (HA) with at least a second polymer preparation (e.g., poloxamer), which may be a solid particle preparation or a liquid preparation, can promote formation of a homogenous polymer solution, as compared to mixing liquid preparations of HA and at least a second polymer.

Accordingly, one aspect provided herein relates to a method of producing a homogenous polymer combination of a hyaluronic acid (HA) polymer preparation and a second polymer preparation. The method comprises a step of combining a HA and a second polymer preparation when the HA polymer preparation is in solid particle form. In some embodiments, a solid particle preparation of HA polymer comprises HA polymer in powder form. One of skill in the art, reading the present disclosure, will understand that HA polymer tends to be hygroscopic; in some embodiments, HA polymer in a solid particle preparation may be or comprise hydrated HA polymer.

In some embodiments, a HA polymer preparation in solid particle form may be combined with at least a second polymer preparation (e.g., poloxamer) in solid particle form (e.g., powder in some embodiments) and then together dissolved concurrently in a liquid solution (e.g., a buffer). In some embodiments, a HA polymer preparation in solid particle form may be combined with at least a second polymer preparation (e.g., poloxamer) in liquid form, which in some embodiments may be a solution of the second polymer in a solvent system (e.g., as described herein).

In some embodiments, such HA and second polymer preparations, and optionally additional polymer preparation(s), are combined under conditions and for a time sufficient so that a homogenous polymer mixture is produced. In some embodiments, such a produced homogenous polymer mixture is characterized in that there is no detectable phase separation observed after maintaining the produced homogenous polymer mixture at a temperature that is below of the critical gelation temperature (e.g., in some embodiments 2-8° C. or in some embodiments at an ambient temperature) for at least 1 hour or longer, including, e.g., at least 2 hours, at least 3 hours, at least 4 hours, at least 5 hours, at least 6 hours, at least 12 hours, at least 18 hours, at least 24 hours, or longer. In some embodiments, such a produced homogenous polymer mixture is characterized in that there is no detectable phase separation observed after maintaining the produced homogenous polymer mixture at a temperature that is below the critical gelation temperature (e.g., in some embodiments 2-8° C. or in some embodiments at an ambient temperature) for at least 1 week or longer, including, e.g., at least 2 weeks, at least 3 weeks, at least 4 weeks, longer. In some embodiments, such a produced homogenous polymer mixture is characterized in that there is no detectable phase separation observed after maintaining the produced homogenous polymer mixture at a temperature that is below the critical gelation temperature (e.g., in some embodiments 2-8° C. or in some embodiments at an ambient temperature) for at least 1 month or longer, including, e.g., at least 2 months, at least 3 months, or longer.

In some embodiments, a HA and a second polymer preparations (e.g., poloxamer), and optionally additional polymer preparation(s), are combined by mixing them at an ambient temperature and/or at a low shear rate. In some embodiments, mixing may be performed by mechanical stirring. As will be understood by one skilled in the art, the shear rate is typically determined by the dimensions of a stirring unit (e.g., a stirred blade or stirrer bar such as a magnetic stirrer bar) and rpm, and the highest shear is typically at the tips of a stirring unit (e.g., stirrer blade or a stirrer bar). In some embodiments, a cylindrical stirrer bar, which induces radial flow, may be used. In some embodiments, an impeller of at least 2 blades (e.g., 2, 3, or 4 blades) may be used to induce axial or radial flow depending on the geometry of the blades. In axial flow, the motion is parallel to the shaft (down and up); in radial flow, the motion is perpendicular to the shaft. In some embodiments, a HA and a second polymer preparations are combined by mixing them at an ambient temperature and at a speed of less than 100 rpm.

In some embodiments, a HA and a second polymer preparations (e.g., poloxamer), and optionally additional polymer preparation(s), are mixed for a period of at least 5 hours, including, e.g., at least 10 hours, at least 15 hours, at least 20 hours, at least 25 hours, at least 30 hours or longer. In some embodiments, a HA and a second polymer preparations, and optionally additional polymer preparation(s) are mixed for a period of 5-30 hours or 10-24 hours.

In some embodiments, a HA and a second polymer preparations (e.g., poloxamer), and optionally additional polymer(s), may be mixed at a temperature of between 2-8° C., for example, in some embodiments for a period of at least 5 hours, including, e.g., at least 10 hours, at least 15 hours, at least 20 hours, at least 25 hours, at least 30 hours or longer.

In some embodiments, a HA and a second polymer preparations (e.g., poloxamer), and optionally additional polymer(s) (e.g., CMCH), are mixed at a temperature of between 2-8° C. and are then quickly brought to a temperature that is at or greater than the respective CGT (e.g., relevant CGTs as described herein) to reach a polymer network state, for example, to prevent phase separation. In some such embodiments, a resulting polymer network may be stored at a temperature that is at or greater than the respective CGT (e.g., relevant CGTs as described herein), for example in some embodiments at an ambient temperature, until it is ready for delivery. In some embodiments, a resulting polymer network may be delivered at a temperature that is lower than the respective CGT (e.g., relevant CGTs as described herein) to render it as a solution and/or liquid preparation.

In some embodiments, a payload (e.g., resiquimod and, optionally, an additional payload) may be incorporated into a homogenous mixture of HA and second polymer preparations. In some embodiments, a payload (e.g., resiquimod and, optionally, an additional payload) may be added by combining a HA and a second polymer preparations with a payload. In some embodiments, a payload (e.g., resiquimod and, optionally, an additional payload) to be combined may be a solid particle preparation. In some embodiments, a payload (e.g., resiquimod and, optionally, an additional payload) to be combined may be a liquid preparation, e.g., a liquid preparation of payload prepared from a resiquimod solid form described herein.

In some embodiments, a produced homogenous polymer mixture (with or without a payload) may be exposed to a gelation temperature that is or above the critical gelation temperature of the polymer mixture for a time sufficient so that a hydrogel is formed. In some embodiments, a produced homogenous polymer mixture (with or without a payload) may be exposed to a gelation temperature of about 35-39° C. In some embodiments, a produced homogenous polymer mixture (with or without a payload) may be exposed to a gelation temperature of about 37° C. In some embodiments, a produced homogenous polymer mixture (with or without a payload) is exposed to a gelation temperature for a period of 5 minutes to 30 minutes.

### Uses of Provided Biomaterial Compositions

Preparations and/or compositions described herein can be useful for various medical applications, including, e.g., but not limited to immunomodulation and/or drug delivery. Thus, in some embodiments, preparations and/or compositions described herein can be formulated into pharmaceutical compositions for administration to subjects in need thereof. Accordingly, in one aspect, provided herein is a method comprising administering to a subject in need thereof a preparation or composition as described and/or utilized herein or a pharmaceutical compositions comprising the same.

In some embodiments, a provided polymer combination preparation and/or composition can be formulated in accordance with routine procedures as a pharmaceutical composition for administration to a subject in need thereof (e.g., as described herein). In some embodiments, such a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, lactose, trehalose, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In some embodiments, a pharmaceutical composition can be sterile. A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a pharmaceutical composition for injection may typically comprise sterile isotonic aqueous buffer. Where necessary, a pharmaceutical composition may also include a local anesthetic to ease pain at a site of injection. In some embodiments, components of a pharmaceutical composition (e.g., as described herein) are supplied separately or mixed together in a single-use form, for example, as a dry lyophilized powder or water free concentrate in a hermetically sealed container such as an ampule or sachet or in a sterile syringe indicating the quantity of a composition comprising a polymer combination preparation (e.g., ones described herein). Where a pharmaceutical composition is to be administered by injection, in some embodiments, a dry lyophilized powder composition comprising a polymer combination preparation (e.g., ones described herein) can be reconstituted with an aqueous buffered solution and then injected to a target site in a subject in need thereof. In some embodiments, a liquid composition comprising a polymer combination preparation (e.g., ones described herein) can be provided in a syringe for administration by injection and/or by a robotic surgical system (e.g., a da Vinci System).

In some embodiments, a liquid composition comprising a polymer combination preparation (e.g., ones described herein) can be provided in a syringe for administration with or without a needle, cannula, or trocar.

In some embodiments, a liquid composition comprising a polymer combination preparation (e.g., ones described herein) can be administered by spraying.

In some embodiments, administration of a liquid composition comprising a polymer combination preparation (e.g., ones described herein) can be gas assisted for use in minimally invasive surgery.

In some embodiments, administration of a liquid composition comprising a polymer combination preparation (e.g., ones described herein) can be achieved by using a multi-barrel syringe, with each barrel containing a separate polymer component preparation, the multiple of which are combined upon depression of the shared plunger.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. For example, such preparatory methods include step of bringing components of a provided polymer combination preparation and resiquimod into association with a diluent or another excipient and/or one or more other accessory ingredients and then, if necessary and/or desirable, shaping and/or packaging the product into a desired single-use unit or multi-use units. Alternatively, such preparatory methods may also include a step of pre-forming a polymer network biomaterial from components of a polymer combination preparation described herein, prior to shaping and/or packaging the product into a desired single-use units or multi-use units.

A pharmaceutical composition in accordance with the present disclosure may be prepared, packaged, and/or sold in bulk, as a single-use unit, and/or as a plurality of single-use units. As used herein, a “single-use unit” is a discrete amount of a pharmaceutical composition described herein. For example, a single-use unit of a pharmaceutical composition comprises a predetermined amount of a composition and/or polymer combination preparation described herein, which in some embodiments can be or comprise a pre-formed polymer network of a polymer combination preparation (e.g., ones described herein), or in some embodiments can be or comprise a liquid or a colloidal mixture of individual components of a polymer combination preparation (e.g., ones described herein).

The relative amount of individual components of a provided polymer combination preparation (e.g., as a pre-formed polymer network biomaterial or as precursor component(s) of such a polymer network biomaterial) and resiquimod, and, optionally, any additional agents in pharmaceutical compositions described herein, e.g., a pharmaceutically acceptable excipient and/or any additional ingredients, can vary, depending upon, e.g., desired material properties of a polymer biomaterial, size of target site, injection volume, physical and medical condition of a subject to be treated, and/or types of cancer, and may also further depend upon the route by which such a pharmaceutical composition is to be administered. In some embodiments, a polymer combination preparation and resiquimod are provided in an effective amount in a pharmaceutical composition to provide a desired therapeutic effect (e.g., but not limited to, inducing anti-tumor immunity in at least one or more aspects, e.g., inducing innate immunity). In some embodiments, a polymer combination preparation and resiquimod are provided in an effective amount in a pharmaceutical composition for treatment of cancer. In some embodiments, a polymer combination preparation and resiquimod are provided in an effective amount in a pharmaceutical composition to inhibit or reduce risk or incidence of tumor recurrence and/or metastasis. In certain embodiments, the effective amount is a therapeutically effective amount of a polymer combination preparation and resiquimod. In certain embodiments, the effective amount is a prophylactically effective amount of a polymer combination preparation and resiquimod.

In certain embodiments, a pharmaceutical composition consists essentially of or consists of a polymer combination preparation (e.g., ones described herein) and resiquimod; to the extent that such a composition may include one or more material(s)/agents other than the polymer combination preparation and resiquimod, such other material(s)/agent(s) do not, individually, or together, materially alter relevant immunomodulatory characteristic(s), e.g., innate immunity modulatory characteristic(s) of the polymer combination preparation and resiquimod.

In certain embodiments, pharmaceutical compositions do not include cells. In certain embodiments, pharmaceutical compositions do not include adoptively transferred cells. In certain embodiments, pharmaceutical compositions do not include T cells. In certain embodiments, pharmaceutical compositions do not include tumor antigens. In certain embodiments, pharmaceutical compositions do not include tumor antigens loaded ex vivo.

In certain embodiments, a pharmaceutical composition is in liquid form (e.g., a solution or a colloid). In certain embodiments, a pharmaceutical composition is in a solid form (e.g., a gel form). In certain embodiments, the transition from a liquid form to a solid form may occur outside a subject's body upon sufficient crosslinking such that the resulting material has a storage modulus consistent with a solid form that allows it to be physically manipulated and implanted in a surgical procedure. Accordingly, in some embodiments, a pharmaceutical composition in a solid form may be amenable for carrying out an intended use of the present disclosure (e.g., surgical implantation). In certain embodiments, the transition from a liquid form to a solid form may occur upon thermal crosslinking in situ (e.g., inside a body of a subject) such that the resulting material has a storage modulus consistent with a solid form. In certain embodiments, a pharmaceutical composition is a suspension.

In some embodiments, a preparation or composition as described and/or utilized herein or a pharmaceutical composition comprising the same may be useful for treatment of cancer. In some such embodiments, a subject to be administered to is a subject suffering from cancer. In some embodiments, a subject to be administered to is a subject suffering from or susceptible to recurrent or disseminated cancer. In some embodiments, a subject to be administered to is a tumor resection subject.

In some embodiments, polymer combination preparations and compositions comprising the same described herein are biocompatible and are useful for various medical applications, e.g., in some embodiments as a drug delivery carrier or formulation (e.g., sustained-release drug delivery composition). For example, in some embodiments, polymer combination preparations and compositions comprising the same described herein are useful for treatment of a disease, disorder, or condition. In some embodiments, polymer compositions and compositions comprising the same described herein are useful for treatment of cancer. In some embodiments, polymer combination preparations and compositions comprising the same described herein are useful to delay the onset of, slow the progression of, or ameliorate one or more symptoms of cancer. In some embodiments, polymer combination preparations and compositions comprising the same described herein are useful to reduce or inhibit primary tumor regrowth. In some embodiments, polymer combination preparations and compositions comprising the same described herein reducing or inhibiting incidence of tumor recurrence and/or metastasis. In some embodiments, polymer combination preparations and compositions comprising the same described herein are useful for inducing anti-tumor immunity.

Accordingly, some aspects provided herein relate to methods of administering to a target site in a subject in need thereof a composition comprising a polymer combination preparation described herein. In some embodiments, a subject receiving such a composition may be carrying a tumor. In some such embodiments, a method comprises intratumoral or peritumoral administration of a composition comprising a polymer combination preparation described herein. In some embodiments, a subject receiving such a composition may be undergoing or may have undergone tumor removal (e.g., by surgical tumor resection). In some embodiments, a subject receiving such a composition may have tumor relapse and/or metastasis. In some such embodiments, a method comprises intraoperative administration of a composition comprising a polymer combination preparation described herein at a tumor resection site of a subject.

In some embodiments, a composition administered to a subject in need thereof comprises a polymer combination preparation and resiquimod. In some embodiments, such a provided composition utilized in methods of the present disclosure may be formulated as a pharmaceutical composition described herein.

In some embodiments, a method comprises administering a provided preparation or composition or a pharmaceutical composition comprising the same at a target site in a tumor resection subject. In some embodiments, such a preparation or composition or a pharmaceutical composition comprising the same is administered at a tumor resection site.

In some embodiments, administration may be performed by implantation. For example, in some embodiments, a preparation or composition comprising a polymer combination preparation in a polymer network state (e.g., a hydrogel) may be administered by implantation.

In some embodiments, administration may be performed by injection. In some embodiments, injection may be performed by a robotic arm. For example, in some embodiments, a preparation comprising a polymer combination preparation in a precursor state (e.g., a liquid state or an injectable state) is administered by injection, wherein the precursor state transitions to a polymer network state (e.g., a more viscous solution or colloid state or a hydrogel) upon the administration.

In some embodiments, administration may be performed concurrently with or subsequent to laparoscopy. In some embodiments, administration may be performed concurrently with or subsequent to a minimally invasive surgery (MIS), e.g., robot-assisted MIS, robotic surgery, and/or laparoscopic surgery, for tumor resection.

In certain embodiments, a method provided herein comprises administering a provided composition to a target site in a subject in need thereof after removal of tumor, for example, after removal of greater than or equal to 50% or higher, by weight, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by weight, of the subject's tumor. In certain embodiments, a method provided herein comprises administering a provided composition to a target site in a subject in need thereof after removal of greater than or equal to 50% or higher, by volume, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by volume, of the subject's tumor. In some embodiments, a method provided herein comprises performing a tumor resection to remove a subject's tumor, prior to administration of a provided composition.

In some embodiments, a composition described and/or utilized herein is administered to a target site in a tumor resection subject immediately after the subject's tumor has been removed by surgical tumor resection. In some embodiments, a composition described and/or utilized herein is intraoperatively administered to a target site in a tumor section subject. In some embodiments, a composition described and/or utilized herein is postoperatively administered to a target site in a tumor resection subject within 24 hours or less, including, e.g., within 18 hours, within 12 hours, within 6 hours, within 3 hours, within 2 hours, within 1 hour, within 30 mins, or less, after the subject's tumor has been removed by surgical tumor resection. In some embodiments, a composition described and/or utilized herein is postoperatively administered one or more times to one or more target sites at one or more time points within 12 months or less from a surgical intervention, including e.g., within 11 months, within 10 months, within 9 months, within 8 months, within 7 months, within 6 months, within 5 months, within 4 months, within 3 months, within 2 months, or within 1 months of a surgical intervention. In some embodiments, a composition described and/or utilized herein is postoperatively administered one or more times to one or more target sites at one or more time points within 31 days, including e.g., within 30 days, within 29 days, within 28 days, within 27 days, within 26 days, within 25 days, within 24 days, within 23 days, within 22 days, within 21 days, within 20 days, within 19 days, within 18 days, within 17 days, within 16 days, within 15 days, within 14 days, within 13 days, within 12 days, within 11 days, within 10 days, within 9 days, within 8 days, within 7 days, within 6 days, within 5 days, within 4 days, within 3 days, within 2 days, or within 1 day of a surgical intervention.

In some embodiments, a target site for administration is or comprises a tumor resection site. In some embodiments, such a tumor resection site may be characterized by absence of gross residual tumor antigen. In some embodiments, such a tumor resection site may be characterized by a negative resection margin (i.e., no cancer cells seen microscopically at the resection margin, e.g., based on histological assessment of tissues surrounding the tumor resection site). In some embodiments, such a tumor resection site may be characterized by a positive resection margin (i.e., cancer cells are seen microscopically at the resection margin, e.g., based on histological assessment of tissues surrounding the tumor resection site). In some embodiments, such a tumor resection site may be characterized by presence of gross residual tumor antigen. In some embodiments, a target site for administration is or comprises a site in close proximity (e.g., within 4 inches, within 3.5 inches, within 3 inches, within 2.5 inches, within 2 inches, within 1.5 inches, within 1 inches, within 0.5 inches, within 0.4 inches, within 0.3 inches, within 0.2 inches, within 0.1 inches or less; e.g., within 10 centimeters, within 9 centimeters, within 8 centimeters, within 7 centimeters, within 6 centimeters, within 5 centimeters, within 4 centimeters, within 3 centimeters, within 2 centimeters, within 1 centimeter, within 0.5 centimeters or less) to a tumor resection site. In some embodiments, a target site for administration is or comprises a sentinel lymph node. In some embodiments, a target site for administration is or comprises a draining lymph node.

As will be understood by one of ordinary skill in the art, compositions that are useful in accordance with the present disclosure can be administered to a target site in subjects in need thereof using appropriate delivery approaches known in the art. For example, in some embodiments, provided technologies can be amenable for administration by injection. In some embodiments, provided technologies can be amenable for administration by minimally invasive surgery (MIS), e.g., robot-assisted MIS, robotic surgery, and/or laparoscopic surgery, which, for example, typically involve one or more small incisions. In some embodiments, provided technologies can be amenable for administration in the context of accessible and/or cutaneous excisions. In some embodiments, provided technologies can be amenable for administration (e.g., by injection) intraoperatively as part of minimally invasive procedure, e.g., minimally invasive surgery (MIS), e.g., robot-assisted MIS, robotic surgery, and/or laparoscopic surgery, and/or procedure that involves one or more accessible and/or cutaneous excisions. In some embodiments, provided technologies can be amenable for administration (e.g., by injection) involving a robotic surgical system (e.g., a da Vinci System), e.g., in some embodiments for minimally invasive administration. For example, in some embodiments, a composition that may be useful for injection and/or in the context of minimally invasive procedure, e.g., minimally invasive surgery (MIS), e.g., robot-assisted MIS, robotic surgery, and/or laparoscopic surgery and/or procedure that involves one or more accessible and/or cutaneous excisions, is liquid, and a polymer combination preparation provided in such a composition is or comprises a polymer solution (e.g., a viscous polymer solution), which upon injection to a target site (e.g., a tumor resection site) in a subject, transitions from a liquid solution state to a polymer network state (e.g., a hydrogel), which in some embodiments, such a transition is triggered by exposure to the body temperature of the subject. In some embodiments, a polymer combination preparation in a pre-formed polymer network biomaterial that is compressible without adversely impacting its structural integrity can be injected, for example, by a minimally invasive procedure, e.g., minimally invasive surgery (MIS), e.g., robot-assisted MIS, robotic surgery, and/or laparoscopic surgery and/or procedure.

In some embodiments, technologies provided herein can be amenable for administration by implantation. For example, in some embodiments, a polymer combination preparation provided in a composition in accordance with the present disclosure is a pre-formed polymer network biomaterial. An exemplary polymer network biomaterial is or comprises a hydrogel. For example, in some embodiments, a provided composition may be administered by surgical implantation to a tumor resection site (e.g., void volume resulting from tumor resection). In some embodiments, a provided composition may be administered by surgical implantation to a tumor resection site and affixed with a bioadhesive. In some embodiments, administration may be performed intraoperatively (i.e., immediately after tumor resection).

In some embodiments, the amount of a polymer combination preparation and/or a therapeutic agent incorporated therein to achieve desirable therapeutic effect(s) such as, e.g., anti-tumor immunity, may vary from subject to subject, depending, for example, on gender, age, and general condition of a subject, type and/or severity of cancer, efficacy of a polymeric biomaterial agonist of innate immunity, and the like.

In some embodiments, the present disclosure provides technologies such that administration of a composition comprising a polymer combination preparation (e.g., ones described herein) is sufficient to provide antitumor immunity and thus does not necessarily require administration of, e.g., a tumor antigen, and/or adoptive transfer of immune cells (e.g., T cells) to a subject in need thereof (e.g., as described herein). Accordingly, in some embodiments, technologies provided herein do not include administering a tumor antigen to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein. In certain embodiments, technologies provided herein do not include adoptive transfer of immune cells (e.g., T cells) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In certain embodiments, the present disclosure provides technologies such that administration of a polymer combination preparation is particularly effective when administered as a co-therapy with e.g., a tumor antigen, and/or adoptive transfer of immune cells (e.g., T cells, NK cells, etc.). In certain embodiments, technologies provided herein include adoptive transfer of immune cells (e.g., T cells, NK cells, etc.) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In some embodiments, technologies provided herein are useful for treatment of cancer in a subject. In some embodiments, technologies provided herein are for use in treatment of a resectable tumor. In some embodiments, technologies provided herein are for use in treatment of a solid tumor (e.g., but not limited to, a blastoma, a carcinoma, a germ cell tumor, and/or a sarcoma). In some embodiments, technologies provided herein are for use in treatment of lymphoma present in a spleen or a tissue outside of a lymphatic system, e.g., a thyroid or stomach.

In some embodiments, technologies provided herein are useful for treating a cancer including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer. In certain embodiments, the cancer is brain cancer. In certain embodiments, the cancer is stomach cancer. In certain embodiments, the cancer is esophageal cancer.

In some embodiments, technologies provided herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing's sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathyroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, a method provided herein may comprise administering to a target site (e.g., as described herein) in a tumor resection subject a provided composition and, optionally, monitoring the tumor resection site or distal sites for risk or incidence of tumor regrowth or tumor outgrowth in the subject after the administration, e.g., every 3 months or longer after the administration, including, e.g., every 6 months, every 9 months, every year, or longer. When the subject is determined to have risk or incidence of tumor recurrence based on the monitoring report, in some embodiments, a subject can be administered a second composition (e.g., as described herein) and/or a different treatment regimen (e.g., chemotherapy).

In some embodiments, technologies provided herein may be useful for treating subjects who are suffering from metastatic cancer. For example, in some embodiments, a method provided herein may comprise administering to a target site (e.g., as described herein) in a subject suffering from one or more metastases who has undergone a tumor resection (e.g., surgical resection of a primary tumor) and, optionally, monitoring at least one metastatic site in the subject after the administration, e.g., every 3 months or longer after the administration, including, e.g., every 6 months, every 9 months, every year, or longer. Based on results of the monitoring report, in some embodiments, a subject can be administered with a second composition (e.g., as described herein) and/or a different treatment regimen (e.g., chemotherapy).

In certain embodiments, the methods described herein do not comprise administering a provided composition prior to tumor resection. In certain embodiments, the methods described herein do comprise administering a provided composition prior to tumor resection. In certain embodiments, technologies provided herein comprise administering a provided composition to a tumor resection site concurrently to tumor resection. In certain embodiments, technologies provided herein comprise administering a provided composition to a tumor resection site following tumor resection.

It will be also appreciated that compositions described herein can be administered in combination with one or more additional pharmaceutical agents and/or therapeutic regimens. For example, in some embodiments, compositions described herein can be administered as part of a combination therapy. For example, compositions can be administered in combination with additional pharmaceutical agents that reduce and/or modify their metabolism, inhibit their excretion, and/or modify their distribution within the body. In some embodiments, a composition as described herein is administered in conjunction with systemic therapies, such as chemotherapy, radiation therapy, and/or immune modulation therapy. In some embodiments, an immune modulation therapy may include systemic and/or localized administration of agents such as small molecules, peptides, proteins, saccharides, steroids, antibodies, fusion proteins, nucleic acid agents (e.g., but not limited to antisense polynucleotides, ribozymes, and small interfering RNAs), peptidomimetics, and the like. For example, in some embodiments, a combination therapy may comprise a composition as described herein and an immune checkpoint inhibition therapy (e.g., via inhibition of PD-1/PD-L1 pathway). In some embodiments, a combination therapy may comprise a composition as described herein and a chemotherapeutic agent. Suitable chemotherapeutic agents can be found among any of a variety of classes of anti-cancer agents including, but not limited to, alkylating agents, anti-metabolites, topoisomerase inhibitors, and/or mitotic inhibitors. In some embodiments, compositions as described herein are administered as part of a combination therapy prior to, during, and/or after, at least one or more additional therapies. It will also be appreciated that the additional therapy employed may achieve a desired effect for the same disorder, and/or it may achieve different effects. In certain embodiments, an additional pharmaceutical agent is not adoptively transferred cells. In certain embodiments, an additional pharmaceutical agent is not T cells. In certain embodiments, an additional pharmaceutical agent is administered multiple days or weeks after administration of a composition described herein.

In some embodiments, polymer preparations provided herein may be useful to provide sustained release of a payload (e.g., resiquimod) incorporated therein.

In certain embodiments, technologies provided herein may be useful for treating subjects who are suffering from a wide array of maladies for which localized agent release may be advantageous. In certain embodiments, technologies provided herein may be used in regenerative medicine. In certain embodiments, technologies provided herein may be used in tissue engineering. In certain embodiments, technologies provided herein may be used to aid in medical imaging (e.g., X-ray, CT scanning, and/or radioisotope imaging). In certain embodiments, technologies provided herein may be used in dentistry (e.g., tooth repair). In certain embodiments, technologies provided herein may be used in dermatological applications (e.g., injections to treat facial wrinkles and or folds). In certain embodiments, technologies provided herein may be used in cosmetic and/or plastic surgery. In certain embodiments, technologies provided herein may be used in orthopedic applications (e.g., bone healing, osteoarthritis, spinal fusion, and/or discs). In certain embodiments, technologies provided herein may be used in the treatment of incontinence and other urological indications (e.g., urinary and/or anal). In certain embodiments, technologies provided herein may be used in the treatment of heart failure. In certain embodiments, technologies provided herein may be used in the treatment of hearing loss. In certain embodiments, technologies provided herein may be used in epidermal and/or internal wound dressing. In certain embodiments, technologies provided herein may be used in the prevention of post-operative adhesion. In certain embodiments, technologies provided herein may be used in cancer immunotherapy, including local extended delivery of immunomodulatory molecules. In certain embodiments, technologies provided herein may be used in treatment of autoimmune and/or rheumatic diseases (e.g., through localized and/or extended delivery of immunomodulatory molecules). In certain embodiments, technologies provided herein may be used in treatment of fibrosis and/or scarring (e.g., through localized and/or extended delivery of anti-fibrotic molecules for the prevention or healing of fibrosis and/or scarring). In certain embodiments, technologies provided herein may be used in treatment of infection (e.g., through localized and/or extended delivery of anti-infective molecules for the prevention and/or treatment of infection, e.g., azithromycin, remdesivir and/or any suitable antibiotics and/or antivirals as known in the art). In certain embodiments, technologies provided herein may be used in pain alleviation (e.g., through localized and/or extended delivery of analgesic molecules for the alleviation of pain, e.g., ketorolac, bupivacaine, and/or any suitable analgesic as known in the art).

In certain embodiments, technologies provided herein may be particularly useful for the extended release of molecules for treatment of ocular pathologies. In certain embodiments, provided technologies may be particularly amenable for intravitreal injection. In certain embodiments, provided technologies may be particularly amenable for topical administration. In certain embodiments, provided technologies may be used for treating glaucoma and/or ocular hypertension (e.g., through localized and/or extended release of beta (adrenergic) blockers, prostaglandin analogs, carbonic anhydrase inhibitors, parasympathetic analogs, alpha 2 adrenergic agonists, Rho kinase inhibitors, and/or docosanoids). In certain embodiments, provided technologies may be used for treating age-related macular degeneration (e.g., through localized and/or extended release of any anti-VEGF agent, VEGF inhibitor, anti-VEGFR agent, and/or VEGFR inhibitor as are known in the art). In certain embodiments, provided technologies may be used for treating symptomatic vitreomacular adhesion (e.g., through localized and/or extended release of ocriplasmin and/or any alpha-2 antiplasmin reducer as known in the art). In certain embodiments, provided technologies may be used for treating post-operative inflammation following any ocular surgery (e.g., through localized and/or extended release of ketorolac, loteprednol, dexamethasone, corticosteroids, and/or any suitable anti-inflammatory agent as known in the art). In certain embodiments, provided technologies may be used to deliver anesthetic agents for ophthalmologic procedures (e.g., localized and/or extended delivery of lidocaine and/or any appropriate anesthetic known in the art). In certain embodiments, provided technologies may be used for treating allergic conjunctivitis via either topical or intracanalicular administration (e.g., through local and/or extended delivery of histamine H1 receptor antagonists and/or dexamethasone). In certain embodiments, provided technologies may be used for treating bacterial conjunctivitis and/or corneal ulcers (e.g., localized and/or extended delivery of fluoroquinolone and/or other suitable antibacterial agents as known in the art). In certain embodiments, provided technologies may be used for treating cystinosis (e.g., localized and/or extended delivery of cystamine hydrochloride and/or other suitable cysteine depleting and/or somatostatin inhibiting agents as known in the art). In certain embodiments, provided technologies may be used for treating neurotrophic keratitis (e.g., localized and/or extended delivery of nerve growth factor and/or other suitable anti-neurotrophic keratitis agents as known in the art). In certain embodiments, provided technologies may be used for treating macular edema following branch or central retinal vein occlusion (e.g., localized and/or extended delivery of dexamethasone and/or other suitable corticosteroid agents as known in the art). In certain embodiments, provided technologies may be used for treating dry eye (e.g., localized and/or extended delivery of cyclosporine and/or other suitable immunomodulatory agents). In certain embodiments, provided technologies may be used for treating HSV-mediated corneal inflammation (e.g., localized and/or extended delivery of trifluridine and/or other suitable antiviral agents as known in the art).

In certain embodiments, a subject being treated is a mammal. In certain embodiments, a subject is a human. In certain embodiments, a subject is a tumor resection human subject. In certain embodiments, a subject is a human subject that is not amenable to tumor resection surgery. In certain embodiments, a subject is a human patient who has received neoadjuvant (pre-operative) therapy. In certain embodiments, a subject is a human patient who has not received neoadjuvant therapy. In certain embodiments, a subject is a human patient who has received neoadjuvant (pre-operative) chemotherapy. In certain embodiments, a subject is a human patient who has not received neoadjuvant (pre-operative) chemotherapy. In certain embodiments, a subject is a human patient who has received neoadjuvant radiation therapy. In certain embodiments, a subject is a human patient who has not received neoadjuvant radiation therapy. In certain embodiments, a subject is a human patient who has received neoadjuvant chemotherapy and radiation therapy. In certain embodiments, a subject is a human patient who has received neoadjuvant molecular targeted therapy. In certain embodiments, a subject is a human patient who has not received neoadjuvant molecular targeted therapy. In certain embodiments, a subject is a human patient who has not received neoadjuvant chemotherapy. In some embodiments, a subject is receiving, has received, or will receive immune checkpoint blockade therapy. In certain embodiments, a subject is receiving immune checkpoint blockade therapy. In certain embodiments, a subject is a human patient who has received and/or is receiving a molecular targeted therapy (e.g., therapies such as those described as neoadjuvants and/or adjuvants) as the sole therapeutic intervention (e.g., a subject for whom surgical resection is not a viable option). In some embodiments, a subject is receiving, has received, or will receive certain other cancer therapeutics (e.g., including but not limited to costimulation, oncolytic virus, CAR T cells, transgenic TCRs, TILs, vaccines, BiTE, ADC, cytokines, modulators of innate immunity, or any combination of these). In certain embodiments, a subject is a human patient who has received neoadjuvant immunotherapy, including immune checkpoint blockade (e.g., anti-CTLA-4, anti-PD-1, and/or anti-PD-L1). In certain embodiments, a subject is a human patient who has not received and/or will not receive neoadjuvant immunotherapy, including immune checkpoint blockade (e.g., anti-CTLA-4, anti-PD-1, and/or anti-PD-L1). In certain embodiments, a subject is a human patient whose tumor has not objectively responded and/or will not objectively respond to neoadjuvant therapy (as defined by Response Evaluation Criteria in Solid Tumors (RECIST) or immune-related Response Criteria (irRC)) (e.g., stable disease, progressive disease). In certain embodiments, a subject is a human patient whose target lesion has objectively responded and/or is objectively responding to neoadjuvant therapy (e.g., partial response, complete response). Non-target lesions may exhibit an incomplete response, stable disease, or progressive disease. In certain embodiments, a subject is a human patient who would be eligible to receive immunotherapy in an adjuvant (post-operative) setting. In certain embodiments, a subject is a domesticated animal, such as a dog, cat, cow, pig, horse, sheep, or goat. In certain embodiments, a subject is a companion animal such as a dog or cat. In certain embodiments, a subject is a livestock animal such as a cow, pig, horse, sheep, or goat. In certain embodiments, a subject is a zoo animal. In another embodiment, a subject is a research animal, such as a rodent, pig, dog, or non-human primate. In certain embodiments, a subject is a non-human transgenic animal such as a transgenic mouse or transgenic pig.

### Kits

The present disclosure also provides kits that find use in practicing technologies as provided herein. In some embodiments, a kit comprises a composition or a pharmaceutical composition described herein and a container (e.g., a vial, ampule, bottle, syringe, and/or dispenser package, or other suitable container). In some embodiments, a kit comprises delivery technologies such as syringes, bags, etc., or components thereof, which may be provided as a single and/or multiple use item. In some embodiments, one or more component(s) of a composition or a pharmaceutical composition described herein are separately provided in one or more containers. For example, individual components of a polymer combination preparation (e.g., ones described herein, for example, but not limited to poloxamer and a second polymer such as hyaluronic acid and/or a chitosan or variants thereof) may be, in some embodiments, provided in separate containers. In some such embodiments, individual components of a biomaterial (e.g., ones described herein, for example, but not limited to poloxamer and a second polymer such as hyaluronic acid and/or a chitosan or variants thereof) may be each provided independently as dry lyophilized powder, dry particles, or a liquid. In some embodiments, individual components of a polymer combination preparation (e.g., ones described herein, for example, but not limited to poloxamer and a second polymer such as hyaluronic acid and/or a chitosan or variants thereof) may be provided as a single mixture in a container. In some such embodiments, a single mixture may be provided as dry lyophilized powder, dry particles, or a liquid (e.g., a homogenous liquid).

In some embodiments, a polymer combination preparation (e.g., ones described herein) may be provided as a pre-formed polymer network biomaterial in a container. In some embodiments, such a pre-formed polymer network biomaterial (e.g., a hydrogel) may be provided in a dried state. In some embodiments, a pre-formed polymer network biomaterial (in a form of a viscous polymer solution) may be provided in a container.

In some embodiments, provided kits may optionally include a container comprising a pharmaceutical excipient for dilution or suspension of a composition or pharmaceutical composition described herein. In some embodiments, provided kits may include a container comprising an aqueous solution. In some embodiments, provided kits may include a container comprising a buffered solution.

In some embodiments, provided kits may comprise resiquimod (e.g., in one or more solid forms described herein). For example, in some embodiments, resiquimod may be provided in a separate container such that it can be added to a polymer combination preparation liquid mixture (e.g., as described herein) prior to administration to a subject. In some embodiments, resiquimod may be incorporated in a polymer combination preparation described herein.

In certain embodiments, a kit described herein further includes instructions for practicing methods described herein. A kit described herein may also include information as required by a regulatory agency such as the U.S. Food and Drug Administration (FDA). In certain embodiments, information included in kits provided herein is prescribing information, e.g., for treatment for cancer. Instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

Other features of the invention will become apparent in the course of the following description of exemplary embodiments, which are given for illustration of the invention and are not intended to be limiting thereof.

### Examples

The following Examples are not intended to limit the scope of any claim. The following non-liming Examples are provided to further illustrate the present teachings. Those of skill in the art, in light of the present application, will appreciate that many changes can be made to the specific embodiments that are provided herein and still obtain a like or similar result without departing from the spirit and scope of the present teachings.

### Example 1. Preparation and Characterization of Resiquimod Solid Forms

### Materials and Methods

X-Ray Powder Diffraction (XRPD): XRPD patterns were collected on a PANalytical DYY884-AERIS-300 diffractometer using the following parameters:

| | |
|---|---|
| Sample stage mode | Not moving |
| Spinner revolution time | None |
| Tube | Cu Anode |
| Incident Beam Path | |
| Soller slit | Soller 0.04 rad |
| Mask | Fixed Mask fixed 13 mm |
| Divergence slit | Fixed slit ⅛° |
| Anti-scatter slit | None |
| Beam knife | Beam knife low |
| Diffracted beam path | |
| Filter | Large beta filter nickel |
| Soller slit | Soller 0.04 rad |
| Anti-Scatter slit | A.S Slit 9 mm |
| Detector | PIXcel 1D-Medipix3 detector |
| Active length (2 Theta) | 5.542 |
| Measurement Parameters | |
| Scan Axis | Gonio |
| Scan mode | Continuous |
| Start angle (°) | 2.99 |
| End angle (°) | 45 |
| Step size (°) | 0.0217329 |
| Time per step(s) | 34.425 |
| Total time (min) | 00:04:55 |
| Generator Settings | 7.5 mA, 40 kV |
| X-ray wavelength (Å) | 1.54056 Å |
| Instrument K value | 0.5 (Kα-2/Kα-1 ratio) |

Differential Scanning Calorimetry (DSC): DSC thermograms were collected on a TA Instruments DSC250 instrument using the following parameters:

| Method | Ramp | |
|---|---|---|
| Sample pan | Aluminium | |
| Heating rate | 10° | C./minute |
| Purge gas | N₂ (50 mL/minute) | |
| Sample size | 2~5 | mg |

Thermogravimetric Analysis (TGA): TGA thermograms were collected on a TA Instruments TGA550 instrument using the following parameters:

| Method | Ramp | |
|---|---|---|
| Sample pan | Platinum | |
| Temperature | RT-350° | C. |
| Heating rate | 10° | C./minute |
| Purge gas at sample | N₂ (60 mL/minute) | |
| Purge gas at balance | N₂ (40 mL/minute) | |
| Sample size | 2~5 | mg |

### Resiquimod Form I

A representative procedure for preparing Resiquimod Form I follows. Resiquimod (10 mg) obtained from anti-solvent recrystallization with DCM and heptane (described below to give Form I) was added to a clear glass vial equipped with a screw thermoset PTFE liner cap and diethyl ether (5 mL) was added. The mixture was vortexed for 1 minute and kept in a ThermoMixer (Eppendorf) for heating and cooling temperature cycles (50-5° C.). The heating was fixed at 50° C. for 6 hours and cooling for 6 hours at 5° C. in ThermoMixer. The heating rate was kept at 3° C./minute and the cooling rate at 1° C./minute. After completion of 5 heat-cool cycles at 1000 RPM, the suspension was centrifuged, and residue was dried under vacuum (−700 mm of Hg) at RT.

The XRPD pattern of Resiquimod Form I is shown in FIG. 1, and the corresponding data are summarized below:

| Pos. [°2θ] | d-spacing [Å] | Rel. Int. [%] |
|---|---|---|
| 8.72 | 10.13 | 100.00 |
| 9.93 | 8.91 | 1.37 |
| 12.24 | 7.23 | 34.99 |
| 13.79 | 6.42 | 1.84 |
| 16.29 | 5.44 | 3.41 |
| 16.96 | 5.23 | 1.00 |
| 17.56 | 5.05 | 22.98 |
| 19.51 | 4.55 | 7.02 |
| 19.97 | 4.45 | 2.15 |
| 21.31 | 4.17 | 6.63 |
| 21.62 | 4.11 | 2.20 |
| 22.00 | 4.04 | 1.14 |
| 23.22 | 3.83 | 1.92 |
| 23.85 | 3.73 | 1.04 |
| 29.15 | 3.06 | 2.66 |

As shown by the DSC curve in FIG. 2, the sample displayed an endotherm with an onset of 193.6° C. and a peak of 195.1° C. FIG. 3 shows the TGA curve of the sample, which displayed a weight loss of 1.985% up to 200° C.

Resiquimod Form I was also prepared by adding resiquimod (10 mg) obtained from anti-solvent recrystallization with DCM and heptane (described below to give Form I) into a clear glass vial, followed by acetone (0.5 mL). The mixture was vortexed for 1 minute and kept in a ThermoMixer (Eppendorf) for heating and cooling temperature cycles (50-5° C.). The heating was fixed at 50° C. for 6 hours and cooling for 6 hours at 5° C. in ThermoMixer. The heating rate was kept at 3° C./minute and the cooling rate at 1° C./minute. After completion of 5 heat-cool cycles at 1000 RPM, the suspension was centrifuged, and residue was dried under vacuum (−700 mm of Hg) at RT.

Resiquimod Form I was also prepared according to the following procedure. A mixture of 2-(ethoxymethyl)-1-(2-hydroxy-2-methylpropyl)-1H-imidazo[4,5-c]quinoline 5-oxide (34.0 kg) in DCM was charged into a reactor, and the mixture cooled to 0-10° C. Trichloroacetyl isocyanate (1.00 kg) was slowly added into the reaction under argon atmosphere, while maintaining a temperature of 0-10° C. The reaction mixture was stirred for 20-30 min at 0-10° C., then allowed to warm to 25-35° C., then to 40-45° C. The reaction mixture was stirred for 1-2 h at 40-45° C. Approx. 3-4 volumes of solvent was then removed under vacuum. Methanol (2.4 L) was added, and then approx. 3-4 volumes of solvent was distilled under vacuum. Methanol (2.4 L) was added again, and then approx. 3-4 volumes of solvent was distilled under vacuum. Next, methanol (16.0 L) was added, followed by a solution of sodium methoxide (5.2 kg) in methanol. The reaction mixture was then heated to 50-55° C. for 1-2 h. Approx. 3-4 volumes of solvent was then distilled off under vacuum and the mixture was cooled to 35° C. DCM (4.0 L) was added and then 3-4 volumes of solvent distilled off under vacuum; the process was repeated twice. DCM (16 L) and water (16 L) were added, the mixture stirred, the organic layer collected. The aqueous layer was extracted two more times with DCM (16 L). The combined organic layers were dried with sodium sulfate and filtered. Approx. 3-4 volume of the filtrate were removed under vacuum and ethyl acetate (2.4 L) was added; this was repeated twice. The mixture was then warmed to 50-55° C., then allowed to cool to 25-35° C. and stirred for 1-2 h. Solids formed and were collected by filtration and dried under vacuum to give crude resiquimod. The crude resiquimod (375 g) was added to dichloromethane (37.5 L) at 25-35° C. The mixture was refluxed for 1-2 h, then cooled to 25-35° C., and filtered. The filtrate was concentrated to a volume of approx. 15 L and then heated to 35-40° C. Heptane (15 L) was then slowly added to the mixture at 35-40° C. The mixture was stirred for 1-2 h at 35-40° C., then cooled to 25-35° C. and stirred for 1-2 h. The resulting solids were collected via filtration and dried under reduced pressure at 50-55° C. to give Resiquimod Form I.

### Resiquimod Form II

A representative procedure for preparing Resiquimod Form II follows. Resiquimod (10 mg) was added to a clear glass vial and methyl isopropyl ketone (0.5 mL) was added. The mixture was vortexed for 1 minute and kept in a ThermoMixer (Eppendorf) for heating and cooling temperature cycles (50-5° C.). The heating was fixed at 50° C. for 6 hours and cooling for 6 hours at 5° C. in ThermoMixer. The heating rate was kept at 3° C./minute and the cooling rate at 1° C./minute. After completion of 5 heat-cool cycles at 1000 RPM, the suspension was centrifuged, and residue was dried under vacuum (−700 mm of Hg) at RT.

The XRPD pattern of Resiquimod Form II is shown in FIG. 4, and the corresponding data are summarized below:

| Pos. [°2θ] | d-spacing [Å] | Rel. Int. [%] |
|---|---|---|
| 7.39 | 11.95 | 5.12 |
| 7.75 | 11.39 | 100.00 |
| 9.65 | 9.16 | 13.77 |
| 9.89 | 8.95 | 2.57 |
| 11.23 | 7.87 | 23.37 |
| 12.25 | 7.22 | 1.80 |
| 14.38 | 6.16 | 6.66 |
| 15.00 | 5.91 | 1.54 |
| 15.50 | 5.72 | 1.93 |
| 16.23 | 5.46 | 3.91 |
| 16.38 | 5.41 | 3.49 |
| 16.80 | 5.28 | 1.72 |
| 18.55 | 4.78 | 1.60 |
| 19.90 | 4.46 | 7.61 |
| 20.80 | 4.27 | 10.89 |
| 22.11 | 4.02 | 2.05 |
| 22.65 | 3.93 | 10.46 |
| 22.90 | 3.88 | 1.20 |
| 23.54 | 3.78 | 5.31 |
| 23.99 | 3.71 | 1.69 |
| 24.29 | 3.66 | 3.70 |
| 25.38 | 3.51 | 1.07 |
| 26.10 | 3.41 | 5.75 |
| 26.61 | 3.35 | 5.95 |
| 27.78 | 3.21 | 2.00 |
| 29.63 | 3.01 | 1.23 |
| 31.04 | 2.88 | 2.62 |
| 32.21 | 2.78 | 2.65 |

As shown by the DSC curve in FIG. 5, the sample displayed a first endotherm with an onset of 140.7° C. and a peak of 145.4° C. and a second endotherm with an onset of 156.7° C. and a peak of 159.7° C. FIG. 6 shows the TGA curve of the sample, which displayed a weight loss of 15.941% up to 170° C.

### Resiquimod Form III

A representative procedure for preparing Resiquimod Form III follows. Resiquimod (10 mg) was added to a clear glass vial and 1,4-dioxane (0.5 mL) was added. The mixture was vortexed for 1 minute and stirred at 50° C. for 7 days. After 7 days, the suspensions were centrifuged, and residues were dried under vacuum (−700 mm of Hg) at RT.

The XRPD pattern of Resiquimod Form III is shown in FIG. 7 (top spectrum), and the corresponding data are summarized below:

| Pos. [°2θ] | d-spacing [Å] | Rel. Int. [%] |
|---|---|---|
| 8.69 | 10.18 | 17.19 |
| 9.18 | 9.64 | 51.47 |
| 9.48 | 9.33 | 23.69 |
| 10.61 | 8.34 | 4.54 |
| 11.97 | 7.40 | 13.68 |
| 12.26 | 7.22 | 3.69 |
| 14.41 | 6.14 | 16.47 |
| 15.47 | 5.73 | 1.50 |
| 16.04 | 5.52 | 3.57 |
| 17.54 | 5.06 | 5.57 |
| 17.95 | 4.94 | 5.04 |
| 18.53 | 4.79 | 100.00 |
| 19.26 | 4.61 | 8.22 |
| 19.70 | 4.51 | 17.63 |
| 20.57 | 4.32 | 5.03 |
| 21.09 | 4.21 | 8.07 |
| 22.10 | 4.02 | 2.32 |
| 22.43 | 3.96 | 1.82 |
| 23.13 | 3.85 | 2.55 |
| 23.76 | 3.74 | 2.40 |
| 24.13 | 3.69 | 5.32 |
| 27.20 | 3.28 | 1.41 |
| 28.00 | 3.19 | 6.78 |
| 28.42 | 3.14 | 3.41 |
| 29.14 | 3.06 | 2.97 |
| 33.06 | 2.71 | 1.15 |
| 34.06 | 2.63 | 1.29 |

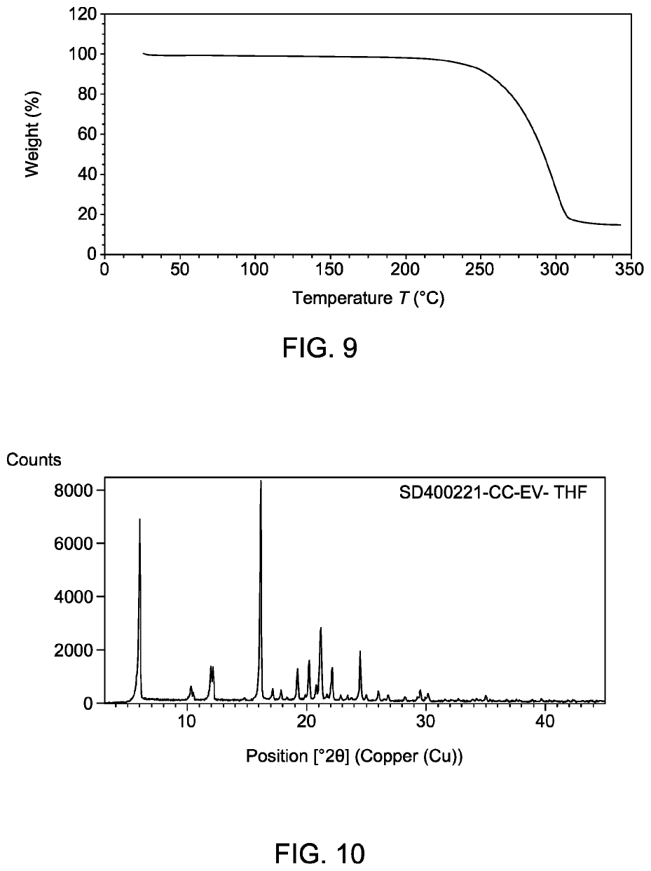

Upon drying, Resiquimod Form III converted to Resiquimod Form I. The dried sample displayed the DSC curve shown in FIG. 8, with an endotherm with an onset of 193.6° C. and a peak of 195.3° C., consistent with conversion into Form I. FIG. 9 shows the TGA curve of the dried sample, which displayed essentially no weight loss up to 200° C., also consistent with conversion into Form I.

### Resiquimod Form IV

A representative procedure for preparing Resiquimod Form IV follows. Resiquimod (15 mg) was added to a clear glass vial and tetrahydrofuran (0.5-1 mL) was added. The mixture was stirred at 40° C. for 4 hours. The mixture was then filtered through a 0.45 μm PVDF filter and the filtrates were kept at 2-8° C. overnight followed by 1 day at −20° C. The solution was allowed to evaporate for 1 day at ambient condition, followed by 2 days under vacuum at RT, and then the solids were collected.

The XRPD pattern of Resiquimod Form IV is shown in FIG. 10, and the corresponding data are summarized below:

| Pos. [°2θ] | d-spacing [Å] | Rel. Int. [%] |
|---|---|---|
| 6.01 | 14.68 | 81.88 |
| 10.30 | 8.59 | 6.20 |
| 10.51 | 8.41 | 3.07 |
| 12.00 | 7.37 | 15.11 |
| 12.15 | 7.28 | 14.51 |
| 14.79 | 5.99 | 1.06 |
| 16.14 | 5.49 | 100.00 |
| 17.16 | 5.17 | 4.48 |
| 17.86 | 4.97 | 4.36 |
| 19.24 | 4.61 | 14.07 |
| 20.21 | 4.39 | 17.07 |
| 20.80 | 4.27 | 5.30 |
| 21.19 | 4.19 | 36.20 |
| 21.70 | 4.10 | 1.94 |
| 22.12 | 4.02 | 14.92 |
| 22.87 | 3.89 | 2.07 |
| 23.42 | 3.80 | 2.10 |
| 23.75 | 3.75 | 1.00 |
| 24.50 | 3.63 | 22.36 |
| 25.01 | 3.56 | 2.38 |
| 26.02 | 3.43 | 3.95 |
| 26.83 | 3.32 | 2.73 |
| 28.23 | 3.16 | 2.00 |
| 29.53 | 3.03 | 5.35 |
| 30.19 | 2.96 | 3.40 |
| 35.02 | 2.56 | 2.51 |

As shown by the DSC curve in FIG. 11, the sample displayed an endotherm with an onset of 193.1° C. and a peak of 194.7° C. FIG. 12 shows the TGA curve of the sample, which displayed a weight loss of 6.965% up to 200° C.

### Resiquimod Form V

A representative procedure for preparing Resiquimod Form V follows. Resiquimod (15 mg) was added to a clear glass vial and methyl ethyl ketone (0.5-1 mL) was added. The mixture was stirred at 40° C. for 4 hours. The mixture was then filtered through a 0.45 μm PVDF filter and the filtrates were kept at 2-8° C. overnight followed by 1 day at −20° C. Crystallized sample was isolated and dried prior to XRPD.

The XRPD pattern of Resiquimod Form V is shown in FIG. 13 (top spectrum), and the corresponding data are summarized below:

| Pos. [°2θ] | d-spacing [Å] | Rel. Int. [%] |
|---|---|---|
| 8.13 | 10.87 | 100.00 |
| 10.20 | 8.68 | 17.80 |
| 10.44 | 8.48 | 11.79 |
| 16.29 | 5.44 | 88.15 |
| 24.56 | 3.62 | 17.67 |
| 32.97 | 2.72 | 2.55 |
| 41.53 | 2.17 | 1.40 |

The sample of Resiquimod Form V was observed to convert into Form I upon drying and/or grinding. See FIG. 13 (bottom three spectra).

As shown by the DSC curve in FIG. 14, the sample displayed a first endotherm with an onset of 55.2° C. and a peak of 62.2° C. and a second endotherm with an onset of 194.5° C. and a peak of 195.4° C. FIG. 15 shows the TGA curve of the sample, which displayed a weight loss of 3.836% up to 110° C.

### Resiquimod Form VI

A representative procedure for preparing Resiquimod Form VI follows. Resiquimod (15 mg) was added to a clear glass vial and anisole (0.5-1 mL) was added. The mixture was stirred at 40° C. for 4 hours. The mixture was then filtered through a 0.45 μm PVDF filter and the filtrates were kept at 2-8° C. overnight followed by 1 day at −20° C. Crystallized samples were isolated and dried prior to obtaining the XRPD.

The XRPD pattern of Resiquimod Form VI is shown in FIG. 16, and the corresponding data are summarized below:

| Pos. [°2θ] | d-spacing [Å] | Rel. Int. [%] |
|---|---|---|
| 9.40 | 9.40 | 100.00 |
| 10.66 | 8.30 | 2.49 |
| 13.02 | 6.80 | 23.99 |
| 13.60 | 6.51 | 11.40 |
| 15.80 | 5.61 | 6.29 |
| 16.35 | 5.42 | 6.31 |
| 16.90 | 5.25 | 10.76 |
| 17.36 | 5.11 | 1.65 |
| 18.13 | 4.89 | 17.15 |
| 18.93 | 4.68 | 47.20 |
| 19.32 | 4.60 | 7.25 |
| 20.38 | 4.36 | 18.79 |
| 21.85 | 4.07 | 3.00 |
| 23.12 | 3.84 | 17.78 |
| 24.14 | 3.69 | 6.05 |
| 24.68 | 3.61 | 3.39 |
| 25.71 | 3.47 | 4.10 |
| 27.78 | 3.21 | 24.88 |
| 28.45 | 3.14 | 7.25 |
| 29.62 | 3.02 | 1.02 |
| 33.68 | 2.66 | 1.86 |
| 36.43 | 2.47 | 5.12 |

As shown by the DSC curve in FIG. 17, the sample displayed a first endotherm with an onset of 72.7° C. and a peak of 78.2° C. and a second endotherm with an onset of 193.7° C. and a peak of 195.1° C. FIG. 18 shows the TGA curve of the sample, which displayed a weight loss of 7.508% up to 110° C.

### Resiquimod Form VII

A representative procedure for preparing Resiquimod Form VII follows. Resiquimod (10 mg) was added to a clear glass vial and dissolved in a minimal volume of 2-methyltetrahydrofuran (0.25-1 mL) with stirring and occasional sonication. The solution was filtered through a 0.45 μm filter and methyl t-butyl ether (3-5 mL) was added slowly with stirring. The mixture was stirred at 2-8° C. for 16 hours. Suspensions were centrifuged, and residues were dried under vacuum (−700 mmHg) at RT.

The XRPD pattern of Resiquimod Form VI is shown in FIG. 19, and the corresponding data are summarized below:

| Pos. [°2θ] | d-spacing [Å] | Rel. Int. [%] |
|---|---|---|
| 6.25 | 14.14 | 100.00 |
| 8.26 | 10.71 | 2.54 |
| 9.92 | 8.91 | 26.51 |
| 10.96 | 8.07 | 10.58 |
| 12.01 | 7.37 | 5.45 |
| 12.35 | 7.17 | 6.94 |
| 12.89 | 6.87 | 8.14 |
| 14.09 | 6.29 | 3.91 |
| 14.64 | 6.05 | 1.80 |
| 15.53 | 5.71 | 2.00 |
| 16.09 | 5.51 | 2.38 |
| 16.51 | 5.37 | 8.69 |
| 17.58 | 5.04 | 3.10 |
| 18.06 | 4.91 | 3.12 |
| 18.99 | 4.67 | 16.59 |
| 20.01 | 4.44 | 6.05 |
| 20.34 | 4.37 | 7.95 |
| 20.78 | 4.28 | 5.49 |
| 21.07 | 4.22 | 6.98 |
| 21.63 | 4.11 | 1.34 |
| 22.10 | 4.02 | 1.72 |
| 23.07 | 3.85 | 6.30 |
| 23.75 | 3.75 | 18.19 |
| 24.24 | 3.67 | 22.37 |
| 24.60 | 3.62 | 4.13 |
| 24.87 | 3.58 | 4.87 |
| 25.59 | 3.48 | 1.12 |
| 26.33 | 3.39 | 4.81 |
| 26.61 | 3.35 | 4.78 |
| 26.92 | 3.31 | 1.46 |
| 27.64 | 3.22 | 5.99 |
| 29.72 | 3.01 | 6.41 |
| 30.53 | 2.93 | 1.94 |
| 31.96 | 2.80 | 1.60 |
| 36.13 | 2.49 | 1.06 |
| 36.63 | 2.45 | 1.75 |
| 37.98 | 2.37 | 1.10 |

As shown by the DSC curve in FIG. 20, the sample displayed a first endotherm with an onset of 95.0° C. and a peak of 108.9° C. and a second endotherm with an onset of 160.9° C. and a peak of 171.8° C. FIG. 21 shows the TGA curve of the sample.

### Example 2. Polymorph Screening of Resiquimod

### Polymorph Screening by Heat-Cool Temperature Cycling

Resiquimod Form I (10 mg) was weighed in a clear glass vial and 0.5 mL of solvent was added. The mixture was vortexed for 1 minute and kept in a ThermoMixer (Eppendorf) for heating and cooling temperature cycles (50-5° C.). The heating was fixed at 50° C. for 6 hours and cooling for 6 hours at 5° C. in ThermoMixer. The heating rate was kept at 3° C./minute and the cooling rate at 1° C./minute. After completion of 5 heat-cool cycles at 1000 RPM, the suspensions were centrifuged, and residues were dried under vacuum (−700 mmHg) at RT. Solutions were evaporated under vacuum (−700 mmHg) at RT. All the solid samples were analyzed by XRPD for evaluation of a new polymorphic form. The results are summarized in Table 1.

TABLE 1

| Solvent | XRPD | Solvent | XRPD |
|---|---|---|---|
| 2-Propanol* | Form I | Anisole | Form I |
| Acetone | Form I | Toluene | Form I |
| Methyl ethyl ketone | Form I | Xylene | Form I |
| Methyl isopropyl ketone | Form II | Pentane | Form I |
| Methyl isobutyl ketone | Form I | Hexane | Form I |
| Methyl acetate | Form I | Heptane | Form I |
| Ethyl acetate | Form I | Cyclohexane | Form I |
| Isopropyl acetate | Form I | 2,2-Dimethoxy propane | Form I |
| Isobutyl acetate | Form I | Octane | Form I |
| Diethyl ether | Form I | Nitromethane | Form I |
| Methyl t-butyl ether | Form I | Acetonitrile | Form I |
| Cyclopentyl methyl ether | Form I | Water | Form I |
| Petroleum Ether | Form I | | |

*Insufficient sample/solution evaporated under vacuum.

### Polymorph Screening by Slurry

Resiquimod Form (10 mg) was weighed in a clear glass vial and 0.5 mL of solvent was added. The mixture was vortexed for 1 minute and left for stirring at 50° C. After 7 days, the suspensions were centrifuged, and residues were dried under vacuum (−700 mmHg) at RT. Solutions were evaporated under vacuum (−700 mmHg) at RT. The results are summarized in Table 2.

TABLE 2

| Solvent | XRPD | Solvent | XRPD |
|---|---|---|---|
| 2-Propanol* | Form I | Anisole | Form I |
| Acetone | Form I | Toluene | Form I |
| Methyl ethyl ketone* | Form I | m-Xylene | Form I |
| Methyl isopropyl ketone* | Form I | Pentane | Form I |
| Methyl isobutyl ketone | Form I | Hexane | Form I |
| Methyl acetate* | Form I | Heptane | Form I |
| Ethyl acetate | Form I | Cyclohexane | Form I |
| Isopropyl acetate | Form I | 2,2-Dimethoxypropane | Form I |
| Isobutyl acetate | Form I | Octane | Form I |
| Diethyl ether | Form I | Nitromethane | Form I |
| Methyl t-butyl ether | Form I | Acetonitrile* | Form I |
| Cyclopentyl methyl ether | Form I | Water | Form I |
| Petroleum Ether | Form I | Dioxane | Form III |

*Insufficient sample/solution evaporated under vacuum.

### Polymorph Screening by Cooling Crystallization

Resiquimod Form I (15 mg) was weighed in a clear glass vial and 0.5-1 mL of solvent was added. The mixture was stirred at 40° C. for 4 hours. The mixtures were then filtered through 0.45 μm PVDF filter and the filtrates were kept at 2-8° C. overnight followed by 1 day at −20° C. Crystallized samples were isolated and dried prior to XRPD. Solutions were left for evaporation 1 day at ambient condition followed by 2 days vacuum evaporation at RT. The results are summarized in Table 3.

TABLE 3

| Solvent | XRPD | Solvent | XRPD |
|---|---|---|---|
| Methanol* | Form I | 2-Methoxethanol* | Form IV |
| Ethanol* | Form I | THF** | Form IV |
| 1-Propanol* | Form I | 2-Methyl THF | No powder |
| | | | obtained |
| 2-Butanol* | Form IV | 1,4-Dioxane* | Form I |
| 1-Butanol* | Form IV | Chloroform** | Form IV |
| Isobutanol* | Form I + Form IV | Dichloromethane** | Form I |
| Amyl alcohol* | Form IV | MEK | Form V |
| Propylene | No powder obtained | Anisole | Form VI |
| glycol | | | |
| 2- | No powder obtained | | |
| Ethoxyethanol | | | |

*Solid obtained upon evaporation under vacuum at RT;
**Solid obtained upon evaporation at RT;
*** Solid obtained upon cooling

### Polymorph Screening by Antisolvent Crystallization

Resiquimod Form I (10 mg) was weighed in a clear glass vial and dissolved in minimum volume (0.25-1 mL) of solvent with stirring and occasional sonication. These solutions were filtered through 0.45 μm filter and 3-5 mL of antisolvent was added slowly with stirring. The mixture was left for stirring at 2-8° C. for 16 hours. Suspensions were centrifuged, and residues were dried under vacuum (−700 mmHg) at RT. Solutions were evaporated under vacuum (−700 mmHg) at RT for 2 days. The results are summarized in Table 4.

TABLE 4

| Solvent | Antisolvent | XRPD |
|---|---|---|
| Methanol | Water | Form I* |
| Ethanol | Water | Form I* |
| 1-Propanol | Water | Form I* |
| 2-Butanol | n-Hexane | Form I* |
| 1-Butanol | n-Hexane | Form I* |
| Isobutanol | n-Hexane | Form I > Form IV* |
| Amyl alcohol | n-Hexane | Form I* |
| Propylene glycol | Water | No powder obtained |
| 2-Ethoxyethanol | Water | No powder obtained |
| 2-Methoxyethanol | Water | Form I* |
| THF | Water | Form I* |
| 2-Methyl THF | MTBE | Form VII |
| 1,4-Dioxane | Water | Form I* |
| Chloroform | n-Hexane | Form IV |
| Dichloromethane | n-Hexane | Form I* |

*Solid obtained upon evaporation under vacuum at RT

### Summary of Resiquimod Solid Forms

Seven solid forms of resiquimod were identified from the polymorph screening described herein. A summary of the forms is provided in Table 5 and Table 6.

TABLE 5

| Form | Process | Solvent |
|---|---|---|
| II | Heat-cool temperature cycle | MIPK |
| III | 50° C. slurry | 1,4-Dioxane |
| IV | Cooling, followed by | 2-Butanol, 1-Butanol, 2- |
| | evaporation under vacuum | Methoxyethanol, Amyl alcohol |
| | Cooling, followed by | THF, Chloroform |
| | evaporation at ambient | |
| | condition | |
| | Antisolvent crystallization | Chloroform/n-Hexane |
| V | Cooling crystallization | MEK |
| VI | Cooling crystallization | Anisole |
| VII | Antisolvent crystallization | 2-Methyl THF/MTBE |

TABLE 6

| Sample | Form | DSC | TGA |
|---|---|---|---|
| Solid obtained from | I | Single melting endotherm onset | No significant weight |
| diethyl ether upon heat- | | 193.59° C. | loss before melting |
| cool crystallization | | | |
| Solid obtained from MIPK | II | Two endotherms; 1^{st} endotherm | 15.91% weight loss |
| upon heat-cool | | onset at 140.67° C.; melting onset | during these thermal |
| crystallization | | at 156.67° C. | events |
| Slurry in dioxane at 50° C. | III | Melting endotherm onset | No significant weight |
| | | 193.62° C. (converted to Form I) | loss before melting |
| | | | (converted to Form I) |
| Solid obtained from THF | IV | Melting endotherm onset | 7.0% weight loss |
| upon cooling followed by | | 193.10° C. | before melting |
| evaporation | | | |
| Crystals obtained from | V | Small endotherm onset 55.21° C. | 3.84% weight loss due |
| MEK cooling | | due to surface coated MEK; | to surface coated MEK |
| crystallization | | melting endotherm onset | |
| | | 194.46° C. | |
| Cooling crystallization | VI | Melting endotherm onset | 7.51% weight loss up |
| from anisole | | 193.72° C. | to 105° C. |
| Solid obtained from 2- | VII | Two broad endotherms onset at | Continuous weight |
| Methyl THF/MTBE upon | | 95.03° C. and 160.92° C. | loss from beginning |
| antisolvent crystallization | | | |

### Example 3. Visual Solubility Assessment of Resiquimod Form I

Solubility was assessed by adding solvent in two steps (0.25 mL per step) to a sample of Resiquimod Form I (10 mg), followed by stirring at room temperature. The results are summarized in Table 7.

TABLE 7

| | Solvent | |
|---|---|---|
| Solvent | added (mL) | Observation |
| Methanol | 0.25-0.5 | Light hazy solution throughout |
| Ethanol | 0.25-0.5 | Light hazy solution throughout |
| 2-Propanol | 0.5 | Suspension |
| 1-Propanol | 0.5 | Light hazy solution |
| 2-Butanol | 0.5 | Light hazy solution |
| 1-Butanol | 0.5 | Light hazy solution |
| Isobutanol | 0.5 | Light hazy solution |
| Amyl alcohol | 0.5 | Light hazy solution |
| Propylene glycol | 0.25-0.5 | Light hazy solution throughout |
| 2-Ethoxyethanol | 0.25-0.5 | Light hazy solution throughout |
| 2-Methoxyethanol | 0.25-0.5 | Light hazy solution throughout |
| Acetone | 0.5 | Suspension |
| MEK | 0.5 | Suspension |
| MIPK | 0.5 | Suspension |
| MIBK | 0.5 | Suspension |
| Methyl acetate | 0.5 | Suspension |
| Ethyl acetate | 0.5 | Suspension |
| Isopropyl acetate | 0.5 | Suspension |
| Isobutyl acetate | 0.5 | Suspension |
| Diethyl ether | 0.5 | Suspension |
| MTBE | 0.5 | Suspension |
| CPME | 0.5 | Suspension |
| Petroleum Ether | 0.5 | Suspension |
| Anisole | 0.5 | Suspension |
| Toluene | 0.5 | Suspension |
| Xylene | 0.5 | Suspension |
| Pentane | 0.5 | Suspension |
| Hexane | 0.5 | Suspension |
| Heptane | 0.5 | Suspension |
| Cyclohexane | 0.5 | Suspension |
| DMP | 0.5 | Suspension |
| Octane | 0.5 | Suspension |
| THF | 0.25-0.5 | Light hazy solution |
| 2-Methyl THF | 0.25 | Clear solution |
| p-Dioxane | 0.5 | Light hazy solution |
| Chloroform | 0.5 | Light hazy solution |
| Dichloromethane | 0.5 | Light hazy solution |
| Nitromethane | 0.5 | Suspension |
| Acetonitrile | 0.5 | Suspension |
| Water | 0.5 | Suspension |

### Example 4. Exemplary Materials and Methods for Preparation and Characterization of Exemplary Polymer Combination Preparations Described Herein and Reference Polymer Biomaterials

The present example relates to the preparation and characterization of exemplary polymer combinations as described herein. In some embodiments, a generality may be observed wherein as the concentration of one biomaterial increases (e.g., poloxamer), the concentration of the at least one additional biomaterial (e.g., hyaluronic acid and/or chitosan/modified chitosan) required to make a suitable polymer network trends towards a decreasing value. In some embodiments, this generality applies in the opposite direction (e.g., suitable polymer networks formed using lower poloxamer concentrations may use higher concentrations of the at least one additional biomaterial).

Exemplary polymer combination preparations comprising poloxamer and hyaluronic acid are shown below:

Preparation comprising 13.5% (w/w) Poloxamer 407 and 0.65% (w/w) 1.5 MDa hyaluronic acid in 0.1 M NaHCO₃, 0.9% saline pH 8.1 or 25 mM phosphate buffer pH 7.4 or pH 8.

Preparation comprising 10-12.5% (w/w) Poloxamer 407 and 0.65-1% (w/w) 1.5 MDa hyaluronic acid in 0.1 M NaHCO₃, 0.9% saline pH 8.1 or 25 mM phosphate buffer pH 7.4 or pH 8.

Preparation comprising 9-10% (w/w) Poloxamer 407 and 1-1.2% (e.g., 1.1%) (w/w) 1.5 MDa hyaluronic acid in 25 mM phosphate buffer pH 7.4 or pH 8.

Preparation comprising 8-9% (w/w) Poloxamer 407 and 1.65-1.75% (w/w) 1.32 MDa hyaluronic acid in 25 mM phosphate buffer pH 7.4 or pH 8.

Preparation comprising 10% (w/w) Poloxamer 407 and 1-1.5% (e.g., 1.3%) (w/w) 773 kDa hyaluronic acid in 10 mM PBS pH 7.4 or 25 mM phosphate buffer pH 7.4 or pH 8.

Preparation comprising 9-10% (w/w) Poloxamer 407 and 1.2-2.5% (w/w) 730 kDa hyaluronic acid in 10 mM PBS pH 7.4 or 25 mM phosphate buffer pH 7.4 or pH 8.

Preparation comprising 9-10% (w/w) Poloxamer 407 and 1.2-2.5% (w/w) 730 kDa hyaluronic acid in 10 mM PBS pH 8 or 25 mM phosphate buffer pH 8.

Preparation comprising 9-11.5% (w/w) Poloxamer 407 and 2-2.75% (w/w) 730 kDa hyaluronic acid in 10 mM PBS pH 7.4 or 25 mM phosphate buffer pH 7.4 or pH 8.

Preparation comprising 12.3% (w/w) Poloxamer 407 and 1.625% (w/w) 730 kDa hyaluronic acid in 25 mM phosphate buffer pH 7.4 or pH 8.

Preparation comprising 8% (w/w) Poloxamer 407 and 1.75%-2.25% (w/w) 337 kDa hyaluronic acid in 25 mM phosphate buffer pH 7.4 or pH 8.

Preparation comprising 10% (w/w) Poloxamer 407 and 2-6% (w/w) 309 kDa hyaluronic acid in 25 mM phosphate buffer pH 7.4 or pH 8.

Preparation comprising 10% (w/w) Poloxamer 407 and 2-6% (w/w) 264-310 kDa hyaluronic acid in 22.5 mM phosphate buffer pH 7.4 or pH 8.

Preparation comprising 8-12.5% (w/w) Poloxamer 407 and 1-4% (w/w) 264-310 kDa hyaluronic acid in 22.5 mM phosphate buffer pH 7.4 or pH 8.

Preparation comprising 8-12.5% (w/w) Poloxamer 407 and 1-4% (w/w) 119 or 120 kDa hyaluronic acid in 25 mM phosphate buffer pH 7.4 or pH 8.

Preparation comprising 10% (w/w) Poloxamer 407 and 2-6% (w/w) 119 or 120 kDa hyaluronic acid in 25 mM phosphate buffer pH 7.4 or pH 8.

Preparation comprising 8-12.5% (w/w) Poloxamer 407 and 1-4% (w/w) 187 kDa hyaluronic acid in 25 mM phosphate buffer pH 7.4 or pH 8.

Preparation comprising 10% (w/w) Poloxamer 407 and 2-6% (w/w) 187 kDa hyaluronic acid in 25 mM phosphate buffer pH 7.4 or pH 8.

Preparation comprising 8-10% (w/w) Poloxamer 338 and 1-1.5% (w/w) 1.32 MDa hyaluronic acid in 25 mM phosphate buffer pH 7.4 or pH 8.

Preparation comprising 8-10% (w/w) Poloxamer 338 and 1.4-2% (w/w) 730 kDa hyaluronic acid in 25 mM phosphate buffer pH 7.4 or pH 8.

Preparation comprising 8-10% (w/w) Poloxamer 338 and 1.75-2.5% (w/w) 119 kDa hyaluronic acid in 25 mM phosphate buffer pH 7.4 or pH 8.

Exemplary polymer combination preparations comprising poloxamer and chitosan or modified chitosan are shown below:

Preparation comprising 13.5% (w/w) Poloxamer 407 and 0.65-1.3% (w/w) carboxymethyl chitosan in 10 mM PBS, 33 mM NaHCO₃, 0.45% saline pH 8.1, or 25 mM phosphate buffer pH7.4.

Preparation comprising 8-12.5% (w/w) Poloxamer 407 and 2.5-5% (w/w) carboxymethyl chitosan in 10 mM PBS, 33 mM NaHCO₃, 0.45% saline pH 8.1, or 25 mM phosphate buffer pH7.4.

Exemplary polymer combination preparations comprising poloxamer, hyaluronic acid, and chitosan or modified chitosan are shown below:

Preparation comprising 8-12.5% (w/w) Poloxamer 407, 2-6% (w/w) 119 kDa hyaluronic acid, and 0.2-5% (w/w) carboxymethyl chitosan in 25 mM phosphate buffer pH7.4.

Preparation comprising 8-12.5% (w/w) Poloxamer 407, 2-6% (w/w) 187 kDa hyaluronic acid, and 0.2-5% (w/w) carboxymethyl chitosan in 25 mM phosphate buffer pH7.4.

Preparation comprising 8-12.5% (w/w) Poloxamer 407, 1-3% (w/w) 773 kDa hyaluronic acid, and 0.1-1% (w/w) carboxymethyl chitosan in 25 mM phosphate buffer pH7.4.

Preparation comprising 8-12.5% (w/w) Poloxamer 407, 1.0-3% (w/w) 730 kDa hyaluronic acid, and 0.1-1% (w/w) carboxymethyl chitosan in 25 mM phosphate buffer pH7.4.

Preparation comprising 6-10% (w/w) Poloxamer 407, 1.25-5% (w/w) 309 kDa hyaluronic acid, and 0.2-1.5% (w/w) carboxymethyl chitosan in 25 mM phosphate buffer pH7.4.

Preparation comprising 6-10% (w/w) Poloxamer 407, 1.25-5% (w/w) 119 kDa hyaluronic acid, and 0.5-2.5% (w/w) carboxymethyl chitosan in 25 mM phosphate buffer pH7.4.

Preparation comprising 8-12.5% (w/w) Poloxamer 407, 1.25-5% (w/w) 119 kDa hyaluronic acid, and 0.2-2% (w/w) carboxymethyl chitosan in 25 mM phosphate buffer pH7.4.

### Rheological Analysis of Exemplary Polymer Combination Preparations:

Rheological analysis of hydrogels formed from polymer combination preparations was performed either using a Rheometric Scientific model SR5 equipped with Peltier system and 25 mm parallel plates or a TA instruments Discovery HR2 rheometer using a 20 mm parallel plate, a 1,500 μm gap, and a frequency sweep of 0.1 Hz to 10 Hz, 0.4% strain at 37° C., soak time of 120 s and run time of 60 s. Maximum storage modulus (Pa) and minimum phase angle 6° were measured.

### Cell Line and Cell Culture:

4T1-Luc2 breast cancer cells were cultured in RPMI-1640 medium, with 10% fetal bovine serum (FBS) and 1% penicillin/streptomycin. All cells were cultured in a 37° C. in a humidified incubator, with 5% Co₂.

### Mouse Tumor Models:

All animal experiments were performed using 6-8 weeks old female BALB/c mice (Jackson Laboratories, #000651). For animal survival studies, 10⁵ 4T1-Luc2 cells were inoculated orthotopically into the fourth mammary fat pad of a mouse. Mice were size-matched and randomly assigned to treatment groups, and surgery was performed on day 10 or day 12 after tumor inoculation. Tumor sizes were measured with calipers. For primary tumor resection, mice were anesthetized with 2% isoflurane, the tumor was resected, and a hydrogel was placed in the surgical site at the time of surgery.

### Exemplary Methods for Hydrogel Preparation:

### (i) Chemically Crosslinked Hyaluronic Acid (HA) Hydrogels:

HyStem HA hydrogels were prepared using the HyStem hydrogel kit (ESI Bio, GS1004). First, 120 μL of Glycosil was added to a Teflon mold (9 mm diameter). Next, 10 μL of an immunomodulatory payload was optionally added and stirred to create a homogeneous mixture. Finally, 30 μL of Extralink was added, and the hydrogel was left to crosslink for one hour.

### (ii) Poloxamer-HA Hydrogels:

Poloxamer-HA hydrogels were prepared by combining appropriate amount of poloxamer (e.g., a solid particle preparation or a liquid preparation) and a solid particle (e.g., powder) preparation of HA in a 4 mL vial (optionally along with 5 μL of 40 mg/mL solution of resiquimod (i.e., R848 (Sigma #SML0196)) in DMSO) to prepare a solution mixture and mixing the solution mixture at 300 rpm for 15 min and then at 100 rpm for overnight. To induce gel formation, the solution mixture was placed in a water bath at 37° C. After 10-15 minutes at 37° C., the sample was observed for gel formation or phase separation (no gel formation). The resulting gels were then subjected to rheological analysis, e.g., as described herein.

In some embodiments, the solution mixture after overnight mixing was then cooled in ice for at least 10 min before transferring 200 μL to a 1 mL syringe (BD-309602) for in vivo administration experiment.

(iii) Poloxamer-CMCH Hydrogels:

Poloxamer-CMCH hydrogels were prepared by weighing appropriate amount of poloxamer and CMCH in an appropriate buffer in a 20 mL vial to prepare a solution mixture and mixing the solution mixture at 300 rpm for 15 min and then at 100 rpm overnight. To induce gel formation, the solution mixture was placed in a water bath at 37° C. After 10-15 minutes at 37° C., the sample was observed for gel formation or phase separation (no gel formation). The resulting gels were then subjected to rheological analysis, e.g., as described herein.

In some embodiments, the solution mixture after overnight mixing was then cooled in ice for at least 10 min before transferring 200 μL to a 1 mL syringe (BD-309602) for in vivo administration experiment.

### Example 5. Gelation Properties of Exemplary Polymer Combination Preparations

The present Example 5 describes gelation properties of certain test polymer combination preparations comprising Poloxamer 407 and a second polymer component, which may be or comprise a carbohydrate polymer (e.g., hyaluronic acid and/or chitosan or a variant thereof).

In many embodiments, polymer combination preparations as described and/or utilized herein are temperature-responsive such that they transition from a precursor state (e.g., a polymer solution or colloid) to a polymer network state in response to a temperature change. In some embodiments, a polymer network state is a more viscous liquid or colloid than the precursor state. In some embodiments, a polymer network state is a hydrogel.

In some embodiments, temperature-responsive polymer combination preparations as described and/or utilized herein transition from a precursor state to a polymer network state at a gelation temperature (e.g., a temperature that is or above the critical gelation temperature of the polymer combination preparation) in the absence of any chemical crosslinkers. In some embodiments, a gelation temperature may be a temperature of 35-39° C. (e.g., at a temperature of 37° C.). In some embodiments, temperature-responsive polymer combination preparations as described and/or utilized herein transition from a precursor state to a polymer network state at the body temperature of a subject (e.g., a human subject) in the absence of any chemical crosslinkers. In some embodiments, temperature-responsive polymer combination preparations as described and/or utilized herein exhibit a sol-gel transition temperature of approximately 28-35° C. or of approximately 20-28° C.

In some embodiments, polymer combination preparations, and/or individual components thereof were prepared in a suitable buffer. In certain embodiments, polymer combination preparations, and/or individual components thereof were prepared in an aqueous buffer system. Examples of suitable aqueous buffer systems at an appropriate pH include, e.g., but are not limited to phosphate buffer and/or bicarbonate buffer at an appropriate pH. In some embodiments, polymer combination preparations, and/or individual components thereof were prepared in phosphate-buffered saline (PBS), sodium phosphate saline (SPS), potassium dihydrogen phosphate buffer, dipotassium hydrogen phosphate buffer, sodium bicarbonate buffer, sodium citrate buffer, sodium acetate buffer, TRIS buffer, and/or HEPES buffer, each at an appropriate pH. In some embodiments, polymer combination preparations, and/or individual components thereof were prepared in an aqueous buffer system at a concentration range of from 1 mM to 500 mM, or from 5 mM to 250 mM, or from 10 mM to 150 mM. In certain embodiments, a suitable aqueous buffer (e.g., a phosphate buffer) was prepared at a concentration of 10 mM-50 mM. In certain embodiments, a suitable aqueous buffer (e.g., a bicarbonate buffer) was prepared at a concentration of 100-200 mM.

In some embodiments, polymer combination preparations, and/or individual components thereof were prepared in a suitable buffer (e.g., ones described herein) with pH around neutral pH. For example, in certain embodiments, polymer combination preparations, and/or individual components thereof can be prepared in a suitable buffer with pH 6-9. In some embodiments, polymer combination preparations, and/or individual components thereof can be prepared in a suitable buffer with pH 7-8. In some embodiments, polymer combination preparations, and/or individual components thereof can be prepared in a suitable buffer with pH 7.2-7.6. In some embodiments, polymer combination preparations, and/or individual components thereof can be prepared in a suitable buffer with pH 7.4. In some embodiments, polymer combination preparations, and/or individual components thereof can be prepared in a suitable buffer with pH 8.0.

To assess gelation properties of various polymer combination preparations, a polymer preparation comprising a poloxamer at a concentration of 12% (w/w) or lower and a second polymer component that is not a poloxamer was exposed to a target temperature for inducing gelation process (e.g., the body temperature of a subject such as a temperature of 37° C.) for a period of time (e.g., about 15-20 minutes) and then the physical state (e.g., solution vs. gel) of the polymer preparation was observed. Qualitative observations were made to determine initial gel formation characteristics. Polymer combination preparations were considered to have formed a “good gel” when the sample becomes translucent or opaque and does not flow when angled or inverted. The sample maintains the shape of the vessel/vial until temperature drops below the CGT. Relatively “weak gels” were qualitatively determined to have more flow when angled or inverted when compared to “good gels” and less flow when compared to solutions below the respective CGT. For polymer preparations that form hydrogels after exposure to the target gelation temperature, rheological analysis was performed, e.g., to determine storage modulus and/or phase angle of the resulting hydrogels.

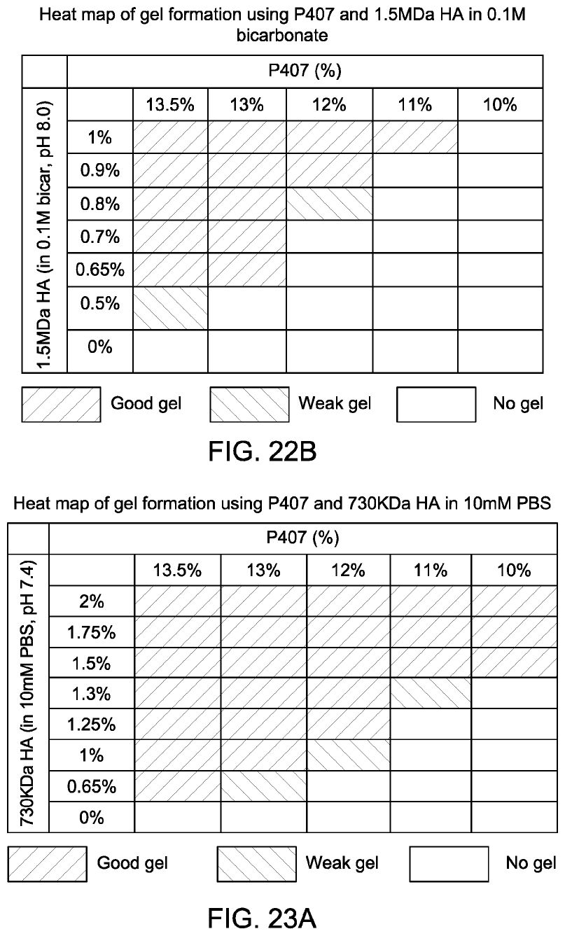

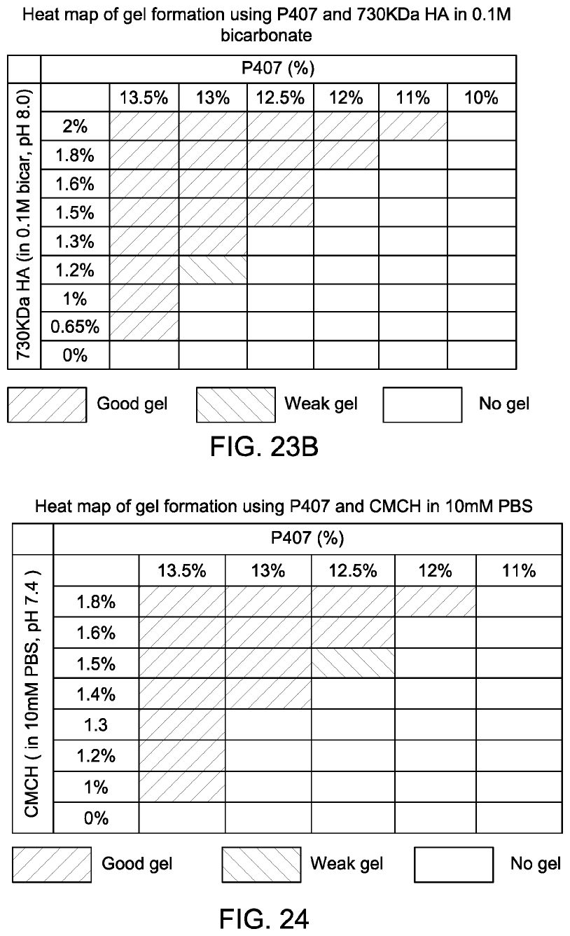

As shown in FIG. 22A, FIG. 22B, FIG. 23A, FIG. 23B, and FIG. 24, various polymer combination preparations comprising Poloxamer 407 (P407) at 9%-13.5% (w/w) and a carbohydrate polymer (e.g., hyaluronic acid or chitosan or modified chitosan) at 0%-2% (w/w) in a phosphate buffer or a bicarbonate buffer (e.g., pH 7-8) were assessed for their gelation properties.

In some embodiments, a carbohydrate polymer included in certain polymer combination preparations is or comprises hyaluronic acid, e.g., having an average molecular weight of 500 kDa-1.5 MDa. In some embodiments, a carbohydrate polymer included in certain polymer combination preparations is or comprises hyaluronic acid having an average molecular weight of 750 kDa. In some embodiments, a carbohydrate polymer included in certain polymer combination preparations is or comprises hyaluronic acid having an average molecular weight of 1.5 MDa. FIG. 22A shows gel formation from certain polymer combination preparations comprising P407 at a concentration of 9.5%-13.5% (w/w) and 1.5 MDa hyaluronic acid at a concentration of 0.65%-1.1% (w/w) in 10 mM PBS, pH 7.4. FIG. 22B shows gel formation from certain polymer combination preparations comprising P407 at a concentration of 11%-13.5% (w/w) and 1.5 MDa hyaluronic acid at a concentration of 0.5%-1% (w/w) in 0.1 M bicarbonate buffer, pH 8. FIG. 23A shows gel formation from certain polymer combination preparations comprising P407 at a concentration of 10%-13.5% (w/w) and 750 kDa hyaluronic acid at a concentration of 0.65%-2% (w/w) in 10 mM PBS, pH 7.4. FIG. 23B shows gel formation from certain polymer combination preparations comprising P407 at a concentration of 11%-13.5% (w/w) and 730 kDa hyaluronic acid at a concentration of 0.65%-2% (w/w) in 0.1 M bicarbonate buffer, pH 8.

In some embodiments, a carbohydrate polymer included in certain polymer combination preparations is or comprises a modified chitosan (e.g., carboxymethyl chitosan; CMCH). FIG. 24 shows gel formation from certain polymer combination preparations comprising P407 at a concentration of 11%-13.5% (w/w) and CMCH at a concentration of 1%-1.8% (w/w) in 10 mM PBS, pH 7.4.

In some embodiments, a carbohydrate polymer included in certain polymer combination preparations is or comprises hyaluronic acid, e.g., having an average molecular weight of 100-900 kDa. In some embodiments, a carbohydrate polymer included in certain polymer combination preparations is or comprises hyaluronic acid having an average molecular weight of about 119 kDa, 187 kDa, 309 kDa, 730 kDa, 773 kDa, 886 kDa or any combination thereof. In some embodiments, such polymer combination preparations as described herein may optionally include modified chitosan.

In some embodiments, a biomaterial polymer combination (e.g., that is a gel at 37° C.) described herein comprises 10% (w/w) poloxamer 407, and 1-2.5% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-4% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 9% (w/w) poloxamer 407, and 3-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 10% (w/w) poloxamer 407, and 2% (w/w) Hyaluronic Acid with a molecular weight of approximately 309 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-4% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 10% (w/w) poloxamer 407, and 3-7% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 9% (w/w) poloxamer 407, and 3-7% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 8% (w/w) poloxamer 407, and 2.5-5% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 8% (w/w) poloxamer 407, and 1.5-2.5% (w/w) Hyaluronic Acid with a molecular weight of approximately 309 kDa, and 1-1.5% (w/w) modified chitosan.

In some embodiments, a biomaterial polymer combination (e.g., that is a gel at 37° C.) described herein comprises 8% (w/w) poloxamer 407, and 1.5% (w/w) Hyaluronic Acid with a molecular weight of approximately 773 kDa, and 0.5-1.0% (w/w) modified chitosan.

In some embodiments, a biomaterial polymer combination (e.g., that is a gel at 37° C.) described herein comprises 11-12% (w/w) poloxamer 407, and 3-5% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 1.5-3% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 5-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 6-8% (w/w) poloxamer 407, and 2-3% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 1-2% (w/w) Hyaluronic Acid with a molecular weight of approximately 700-800 kDa, and optionally 0.1-1% (w/w) modified chitosan.

In some embodiments, a biomaterial polymer included in certain polymer combination preparations is or comprises combinations as represented in Table 8, Table 9, Table 10, or Table 11.

TABLE 8
Gelation Properties of Certain Biomaterial Combinations

| MW HA kDa | wt % HA | wt % P407 | wt % CMCH | Gel at 37° C. |
|---|---|---|---|---|
| — | — | 8 | 0.4-2.5 | No |
| — | — | 9 | 0.4-2.5 | No |
| 119 | 2.5-5 | 8 | — | Yes |
| 119 | 3 | 8 | 0.4-0.5 | No |
| 119 | 4-5 | 8 | 0.4-0.5 | Yes |
| 119 | 3-7 | 9 | — | Yes |
| 119 | 3 | 9 | 0.4-0.5 | No |
| 119 | 3-4 | 10 | — | Yes |
| 119 | 3-4 | 10 | 0.5-2.5 | Yes |
| 119 | 3-6 | 11 | — | Yes |
| 119 | 3-4 | 11.5 | — | Yes |
| 187 | 3-4 | 8 | — | Yes |
| 187 | 3-4.1 | 10 | — | Yes |
| 309 | 1.5-2.5 | 8 | — | Yes |
| 309 | 1.5-2.5 | 8 | 1-1.5 | Yes |
| 309 | 2-3 | 10 | — | Yes |
| 730 | 2.7 | 6 | — | Yes |
| 730 | 2.5 | 7 | — | Yes |
| 730 | 2.3 | 8 | — | Yes |
| 730 | 2.2-2.5 | 9 | — | Yes |
| 730 | 1.2-3 | 10 | — | Yes |
| 730 | 1.8-2 | 11 | — | Yes |
| 773 | 2.7 | 6 | — | Yes |
| 773 | 2.5 | 7 | — | Yes |
| 773 | 1.5-2.3 | 8 | — | Yes |
| 773 | 1.2-2 | 10 | — | Yes |
| 773 | 1.3 | 10 | 0.5 | Yes |
| 886 | 1.8 | 9 | — | Yes |
| 886 | 1.5 | 11 | — | Yes |

Formulations comprising polymer combinations as described in Table 9, Table 10, and Table 11 were tested for gelation characteristics and were found to form gels at 37° C. In some embodiments, a polymer combination preparation described herein was considered as forming a gel when the polymer combination preparation changed from a transparent solution to an opaque composition, when the composition was observed to have no flow, and/or when a magnetic stir bar present in the polymer combination preparation did not move in the presence of a magnetic field.

TABLE 9
Compositions comprising low MW HA,
demonstrated to form a gel at 37° C.

| wt % | wt % | Mw HA | wt % | wt % | wt % | Mw HA | wt % |
|---|---|---|---|---|---|---|---|
| P407 | HA | kDa | CMCH | P407 | HA | kDa | CMCH |
| 10 | 5 | 73 | — | 10 | 3 | 187 | 0.2 |
| 10 | 7.5 | 73 | — | 10 | 3 | 187 | 0.3 |
| 6 | 9 | 119 | — | 10 | 3 | 187 | 2.5 |
| 8 | 2.5 | 119 | — | 10 | 4 | 187 | 0.2 |
| 8 | 3 | 119 | — | 10 | 4 | 187 | 0.3 |
| 8 | 3.7 | 119 | — | 6 | 2.8 | 309 | — |
| 9 | 6 | 119 | — | 8 | 2.25 | 309 | — |
| 9 | 6.5 | 119 | — | 8 | 2.6 | 309 | — |
| 9 | 7 | 119 | — | 8 | 2.7 | 309 | — |
| 10 | 1.95 | 119 | — | 8 | 2.8 | 309 | — |
| 10 | 2 | 119 | — | 9 | 2.5 | 309 | — |
| 10 | 3 | 119 | — | 9 | 2.6 | 309 | — |
| 10 | 4 | 119 | — | 9 | 2.7 | 309 | — |
| 10 | 4.1 | 119 | — | 9 | 2.8 | 309 | — |
| 11 | 1.75 | 119 | — | 10 | 1.5 | 309 | — |
| 11 | 5 | 119 | — | 10 | 2 | 309 | — |
| 11 | 5.5 | 119 | — | 10 | 2.05 | 309 | — |
| 11 | 6 | 119 | — | 10 | 2.2 | 309 | — |
| 11.5 | 3 | 119 | — | 10 | 2.3 | 309 | — |
| 11.5 | 4 | 119 | — | 10 | 2.4 | 309 | — |
| 12 | 1.15 | 119 | — | 10 | 2.5 | 309 | — |
| 8 | 2.5 | 119 | 1.5 | 10 | 2.56 | 309 | — |
| 8 | 4 | 119 | 0.3 | 10 | 2.6 | 309 | — |
| 8 | 4 | 119 | 0.4 | 10 | 2.7 | 309 | — |
| 8 | 5 | 119 | 0.3 | 10 | 2.8 | 309 | — |
| 8 | 5 | 119 | 0.4 | 10 | 3 | 309 | — |
| 10 | 3 | 119 | 0.15 | 10.4 | 2.4 | 309 | — |
| 10 | 3 | 119 | 0.2 | 11 | 1 | 309 | — |
| 10 | 3 | 119 | 0.3 | 11 | 2.4 | 309 | — |
| 10 | 3 | 119 | 0.4 | 11 | 2.5 | 309 | — |
| 10 | 3 | 119 | 0.5 | 11 | 2.6 | 309 | — |
| 10 | 3 | 119 | 1 | 11 | 2.8 | 309 | — |
| 10 | 3 | 119 | 1.5 | 11 | 3.3 | 309 | — |
| 10 | 3 | 119 | 2 | 11 | 3.5 | 309 | — |
| 10 | 3 | 119 | 2.5 | 11.05 | 2.4 | 309 | — |
| 10 | 4 | 119 | 0.3 | 11.2 | 2.2 | 309 | — |
| 10 | 4 | 119 | 0.4 | 11.25 | 2.2 | 309 | — |
| 10 | 4 | 119 | 0.5 | 11.25 | 2.3 | 309 | — |
| 10 | 4 | 119 | 1 | 11.25 | 2.4 | 309 | — |
| 10 | 4 | 119 | 1.5 | 11.25 | 2.5 | 309 | — |
| 10 | 4 | 119 | 5 | 11.5 | 2 | 309 | — |
| 9 | 6.5 | 119/187 | — | 12 | 0.7 | 309 | — |
| 11 | 5.5 | 119/187 | — | 8 | 2 | 337 | — |
| 11.25 | 2.8 | 144 | — | 8 | 2.5 | 337 | — |
| 11.5 | 2.4 | 144 | — | 10 | 1.75 | 337 | — |
| 11.5 | 2.5 | 114 | — | 11 | 2.4 | 309 | — |
| 11.5 | 2.6 | 144 | — | 11 | 2.5 | 309 | — |
| 11.5 | 2.7 | 144 | — | 11 | 2.6 | 309 | — |
| 11.5 | 2.8 | 144 | — | 11 | 2.8 | 309 | — |
| 8 | 3 | 187 | — | 11 | 3.3 | 309 | — |
| 8 | 4 | 187 | — | 11 | 3.5 | 309 | — |
| 9 | 5.5 | 187 | — | 11.05 | 2.4 | 309 | — |
| 10 | 3 | 187 | — | 11.2 | 2.2 | 309 | — |
| 10 | 3.08 | 187 | — | 11.25 | 2.2 | 309 | — |
| 10 | 4 | 187 | — | 11.25 | 2.3 | 309 | — |
| 10 | 4.1 | 187 | — | 11.25 | 2.4 | 309 | — |
| 11 | 4.5 | 187 | — | 11.25 | 2.5 | 309 | — |
| 11.25 | 2.8 | 187 | — | 11.5 | 2 | 309 | — |
| 11.25 | 3 | 187 | — | 12 | 0.7 | 309 | — |

TABLE 10
Compositions comprising high MW HA -
demonstrated to form a gel at 37° C.

| wt % P407 | wt % HA | Mw HA kDa |
|---|---|---|
| 8 | 1.6 | 730 |
| 8 | 2 | 730 |
| 8 | 2.25 | 730 |
| 8.5 | 1.6 | 730 |
| 9 | 1.6 | 730 |
| 9 | 2.2 | 730 |
| 9.5 | 1.6 | 730 |
| 10 | 1.2 | 730 |
| 10 | 1.3 | 730 |
| 10 | 1.4 | 730 |
| 10 | 1.5 | 730 |
| 10 | 1.6 | 730 |
| 10 | 1.8 | 730 |
| 10 | 2 | 730 |
| 10 | 2.25 | 730 |
| 10 | 2.5 | 730 |
| 11.5 | 1.6 | 730 |
| 12.3 | 1.625 | 730 |
| 12.5 | 1.26 | 730 |
| 12.5 | 1.6 | 730 |
| 6 | 2.7 | 731 |
| 7 | 2.5 | 731 |
| 8 | 2.3 | 731 |
| 9 | 2.2 | 731 |
| 9 | 2.5 | 731 |
| 10 | 2 | 731 |
| 10 | 2.25 | 731 |
| 11 | 1.8 | 731 |
| 11 | 2 | 731 |
| 6 | 1.95 | 766 |
| 8 | 1.65 | 766 |
| 9 | 2.2 | 766 |
| 10 | 1.3 | 766 |
| 10 | 2 | 766 |
| 11 | 1.2 | 766 |
| 11 | 1.5 | 766 |
| 11 | 1.6 | 766 |
| 11 | 1.7 | 766 |
| 11 | 1.8 | 766 |
| 11.25 | 1.8 | 766 |
| 12 | 0.7 | 766 |
| 6 | 2.4 | 773 |
| 6 | 2.7 | 773 |
| 7 | 2.5 | 773 |
| 8 | 2.3 | 773 |
| 9 | 2.2 | 773 |
| 10 | 1.2 | 773 |
| 10 | 1.3 | 773 |
| 10 | 1.4 | 773 |
| 10 | 2 | 773 |
| 10 | 2.4 | 773 |
| 11 | 1.8 | 773 |
| 9 | 1.8 | 866 |
| 11 | 1.5 | 866 |
| 6 | 1.85 | 1320 |
| 8 | 1.5 | 1320 |
| 10 | 1.25 | 1320 |
| 11 | 0.9 | 1320 |
| 12 | 0.7 | 1320 |
| 8 | 1.1 | 1500 |
| 8 | 1.5 | 1500 |
| 9.9 | 1.0 | 1500 |
| 9.9 | 1.5 | 1500 |
| 9.9 | 1.1 | 1500 |
| 10 | 1.0 | 1500 |
| 10 | 1.1 | 1500 |
| 10 | 1.25 | 1500 |
| 10 | 1.5 | 1500 |

TABLE 11
Compositions comprising poloxamer 338 -
demonstrated to form a gel at 37° C.

| wt % P338 | wt % HA | Mw HA kDa |
|---|---|---|
| 8 | 3 | 119 |
| 8 | 2 | 337 |
| 8 | 1.6 | 730 |
| 8 | 1.5 | 1500 |
| 10 | 2.5 | 119 |
| 10 | 1.75 | 337 |
| 10 | 1.4 | 730 |
| 10 | 1.1 | 1500 |

### Example 6. Rheological Properties of Exemplary Polymer Combination Preparations

The present Example 6 describes rheological properties of certain test polymer combination preparations as described in Example 4 and/or Example 5 above, as compared to those of reference chemically-crosslinked polymer biomaterials. Specifically, Example 6 describes storage modulus of certain test polymer combination preparations as described in Example 4 and/or Example 5 above, as compared to that of chemically-crosslinked hyaluronic acid biomaterials. As will be understood by a skilled artisan, methods for measuring storage modulus of biomaterials are known in the art. For example, in some embodiments, storage modulus of test and control biomaterials were measured using a rheometer with a parallel plate (e.g., a TA instruments Discovery HR2 rheometer using a 20 mm parallel plate, a 1,500 μm gap) at a frequency sweep of 0.1 Hz to 10 Hz, 0.4% strain at 37° C., soak time of 120 s and run time of 60 s. Results of storage modulus of certain test biomaterials are shown in Table 12 below:

TABLE 12
Certain Storage Modulus of Certain Biomaterial Combinations

| Polymer biomaterial | Storage modulus (G′, Pa) |
|---|---|
| 18% P407 | 15,750 |
| 13.5% P407 + 0.65% HA (10 mM PBS) | 8,200 |
| 13.5% P407 + 0.65% HA (0.1M bicarbonate) | 7,800 |
| 13.5% P407 + 1.3% CMCH (10 mM PBS) | 900 |
| 10% P407 + 1% HA (10 mM PBS) | 200 |
| HyStem 12.5% Extralink | 1,000 |

In some embodiments, the storage modulus of hydrogels formed from exemplary polymer combination preparations (e.g., ones described herein) was not significantly different from the storage modulus of control 18% (w/w) P407 hydrogels at 37° C. In some embodiments, the storage modulus of hydrogels formed from exemplary polymer combination preparations (e.g., ones described herein) was about half of that of control 18% (w/w) P407 hydrogels. In some embodiments, the storage modulus of hydrogels formed from exemplary polymer combination preparations (e.g., ones described herein) was less than about 1/10th of control 18% (w/w) P407 hydrogels at 37° C. In some embodiments, the storage modulus of hydrogels formed from exemplary polymer combination preparations (e.g., ones described herein) was about less than 1/100th that of control 18% (w/w) P407 hydrogels at 37° C. In specific embodiments, the storage modulus of hydrogels formed from exemplary polymer combination preparations (e.g. ones described herein) was about 8-10 kPa, about 7-9 kPa, about 6-8 kPa, about 5-7 kPa, about 4-6 kPa, about 3-5 kPa, about 2-4 kPa, about 1-3 kPa, about 500 Pa-2 kPa, about 1 kPa, or less than 1 kPa.

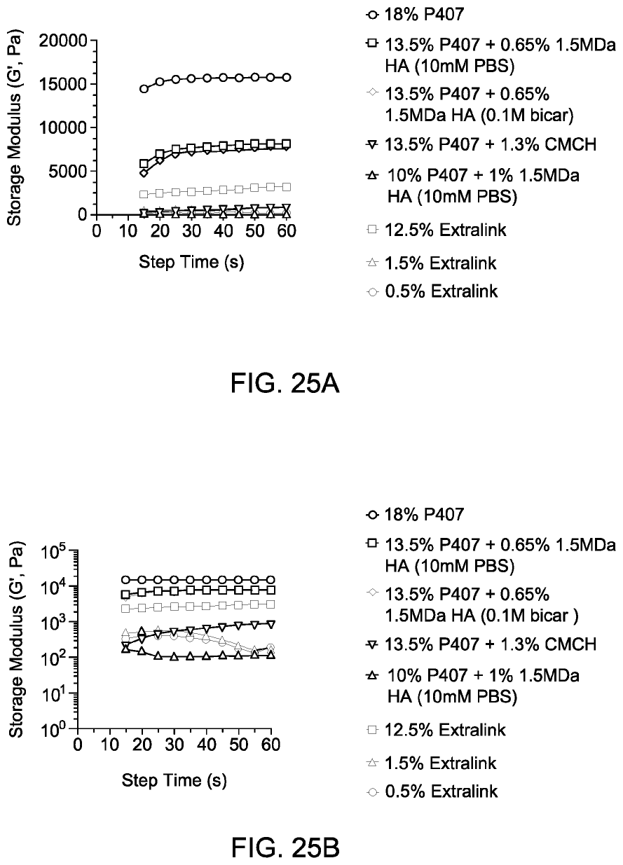

As shown in FIG. 25A and FIG. 25B, hydrogels formed from polymer combination preparations of P407 at a concentration of 13.5% or lower with hyaluronic acid or carboxymethyl chitosan have a lower storage modulus, e.g., by at least 30% or more, than that of a hydrogel formed from P407 at a concentration of 18% (w/w). FIG. 25A and FIG. 25B show hydrogels formed from polymer combination preparations of 10% P407 and 1% HA (1.5 MDa) or of 13.5% P407 and 1.3% carboxymethyl chitosan have a lower storage modulus, e.g., by at least 10% or more, than that of a chemically-crosslinked hyaluronic acid hydrogel with 12.5% Extralink thiol crosslinker (“HyStem”).

The storage stability of certain polymer combination preparations (e.g., ones described herein) were also assessed. For example, to assess the storage stability of polymer biomaterials, their storage moduli were measured over a period of time.

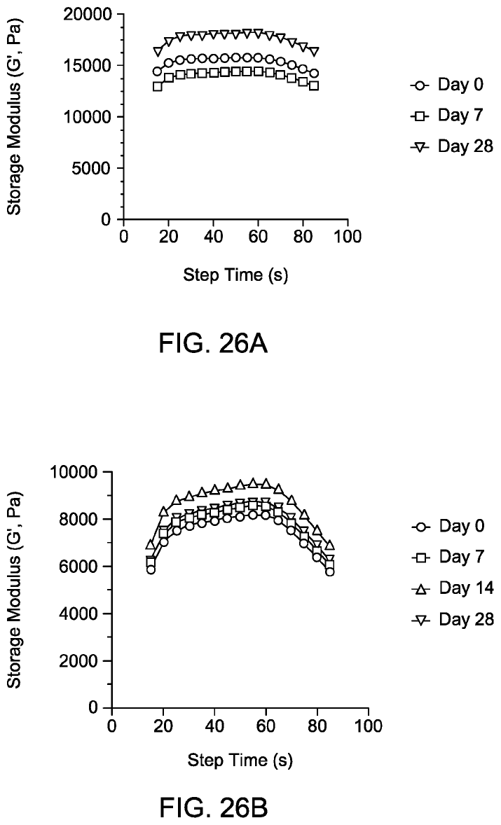

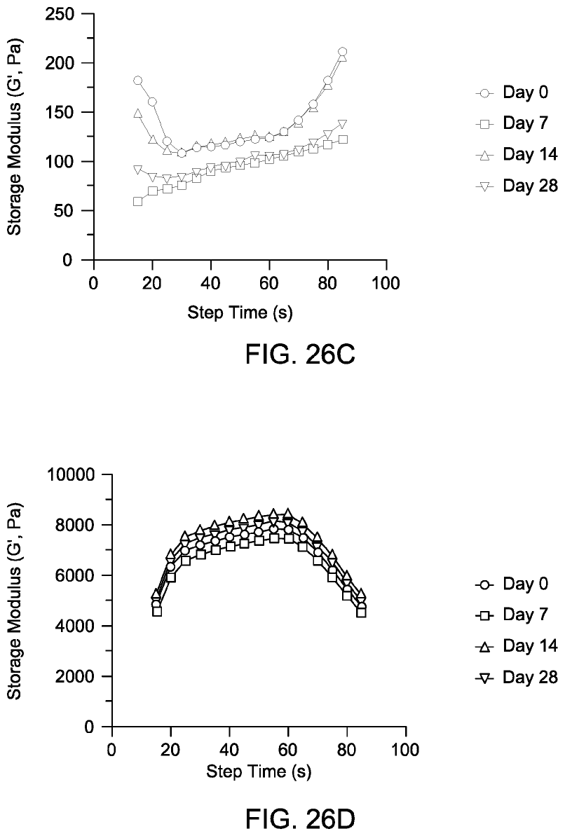

As shown in FIG. 26A, FIG. 26B, FIG. 26C, and FIG. 26D, the storage stability of hydrogels formed from certain polymer combination preparations (e.g., ones described herein) are comparable to that of a reference biomaterial, e.g., a hydrogel formed from 18% (w/w) P407, as demonstrated by no significant change in their storage modulus over a period of about 1 month. In some embodiments, the storage modulus of hydrogels formed from exemplary polymer combination preparations (e.g. ones described herein) was largely stable over time. In some embodiments, the storage modulus of hydrogels formed from exemplary polymer combination preparations (e.g. ones described herein) was stable for one week, two weeks, three weeks, four weeks, or greater than four weeks.

### Example 7. In Vivo Assessment of Exemplary Polymer Composition Preparations for Treatment of Tumor Resection Subjects

The present Example 7 demonstrates in vivo efficacy of certain polymer combination preparations comprising Poloxamer 407 and a second polymer component, which may be or comprise a carbohydrate polymer (e.g., hyaluronic acid and/or chitosan or a variant thereof) administered in tumor resection subjects (e.g., at a tumor resection site). In some embodiments, such polymer combination preparations may be incorporated with an immunomodulatory payload (e.g., resiquimod).

In some embodiments, a provided composition comprising a polymer combination preparation and resiquimod is considered and/or determined to be useful for treatment of cancer (including, e.g., prevention or reduction in the likelihood of tumor relapse or metastasis) in accordance with the present disclosure when such a composition, after administration at a tumor resection site, reduces incidence of tumor recurrence and/or metastasis after the tumor resection (e.g., at least 1 month after tumor resection when the test subject is a mouse subject, or at least 3 months after tumor resection when the test subject is a human subject), for example, by at least 10% or more (comprising, e.g., at least 20%, at least 30%, at least 40%, at least 50%, at least 60%, at least 70%, at least 80%, at least 90%, or more), as compared to that which is observed when such a composition is not administered, or is administered without incorporation of resiquimod.

In some embodiments, female BALB/cJs mice were inoculated orthotopically with 100,000 breast cancer cells (e.g., 4T1-Luc2 cells). Ten days later, tumors were surgically resected, and either (i) a composition described herein (e.g., comprising polymer combination preparation and resiquimod, or (ii) a control composition (e.g., comprising polymer combination preparation without resiquimod) was placed at a tumor resection site.

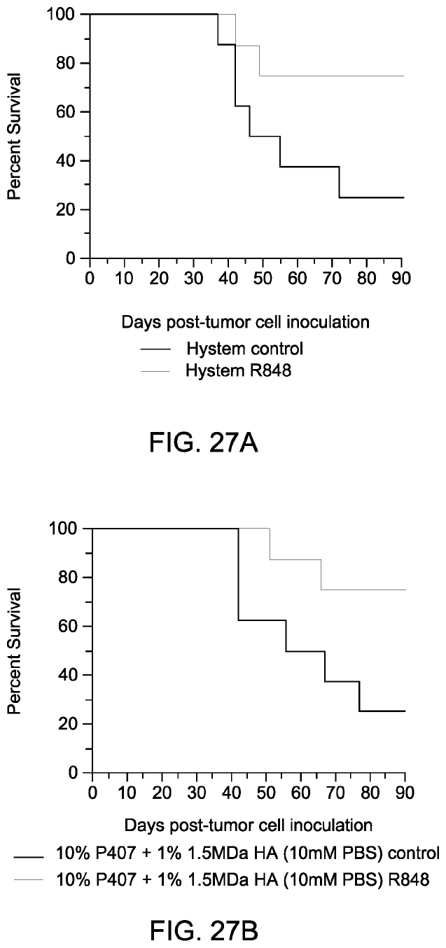

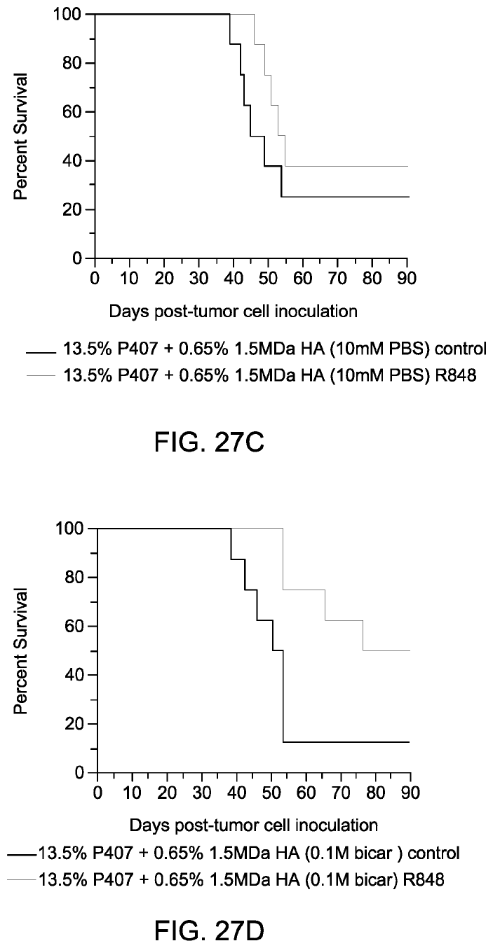

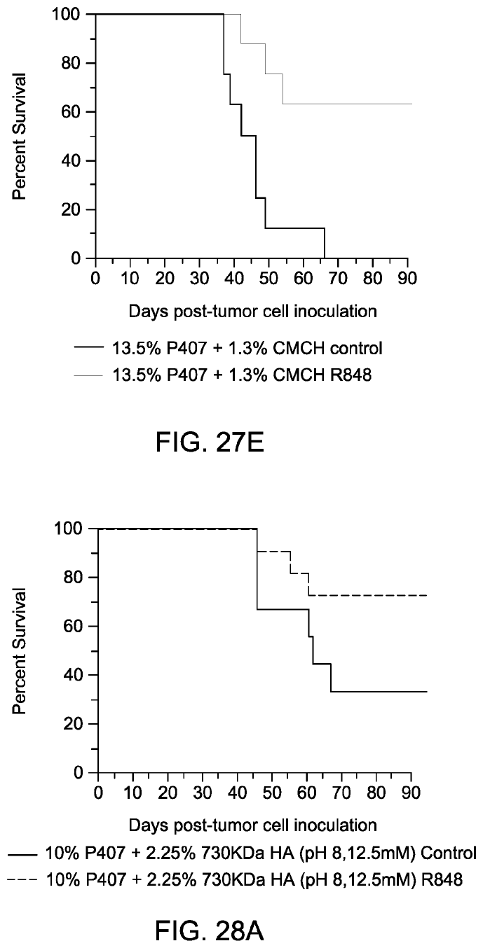

As shown in FIG. 27A, FIG. 27B, FIG. 27C, FIG. 27D, and FIG. 27E, the group of tumor resection mice receiving a crosslinked hydrogel combination of 8-13.5% (w/w) or 6-11% (w/w) Poloxamer (e.g., 10% poloxamer e.g., poloxamer P407) and 0.6-1.5% (w/w) HA (e.g., 1% HA 1.5 MDa), incorporated with resiquimod (R848), at a tumor resection site survived over a longer period of time, e.g., by at least 50% or more, as compared to the control group receiving the crosslinked hydrogel combination without resiquimod. In addition, the group of tumor resection mice receiving such a crosslinked hydrogel combination incorporated with resiquimod exhibited a much higher survival rate than the control group receiving the crosslinked hydrogel combination without resiquimod. Further, the efficacy of such a crosslinked hydrogel combination of 8-13.5% (w/w) Poloxamer (e.g., 10% poloxamer e.g., poloxamer P407) and 0.6-1.5% (w/w) HA (e.g., 1% HA 1.5 MDa), incorporated with resiquimod was comparable to or better than that was observed with a chemically-crosslinked hyaluronic acid hydrogel (“HyStem”) incorporated with resiquimod.

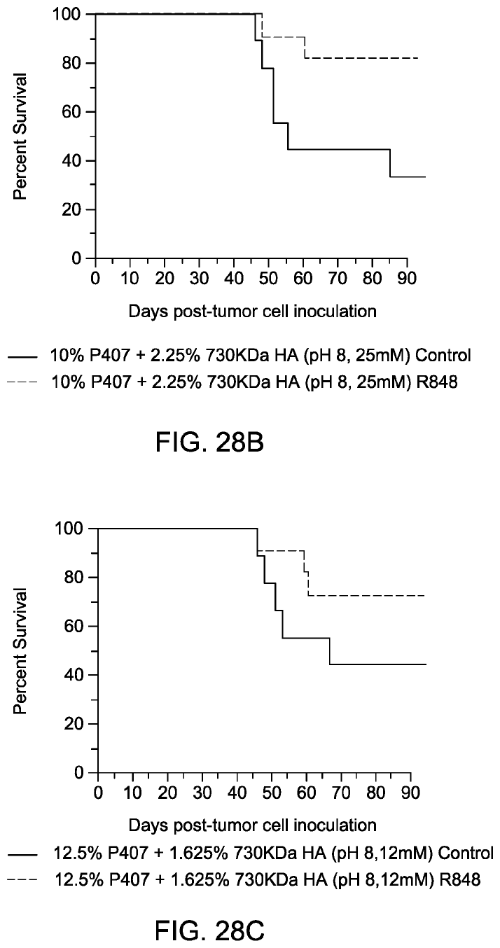

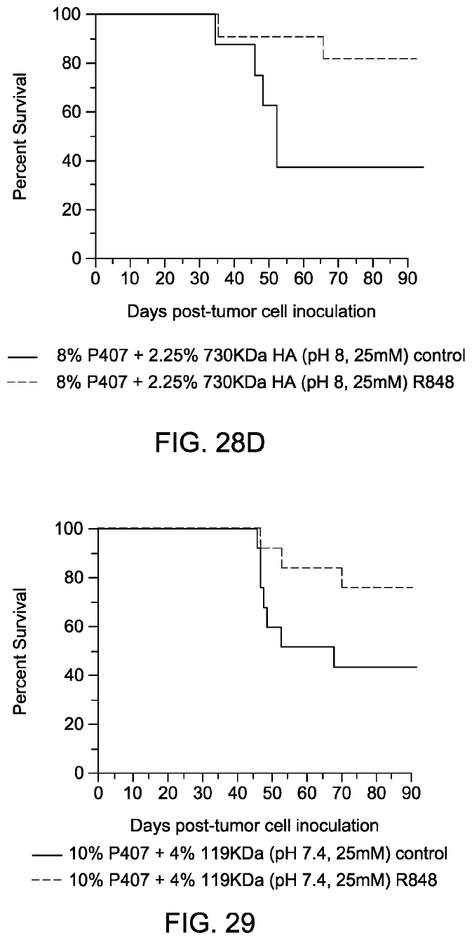

As shown in FIG. 28A, FIG. 28B, FIG. 28C, and FIG. 28D the group of tumor resection mice receiving an immunomodulatory polymer combination of 8-12.5% (w/w) or 6-11% (w/w) poloxamer (e.g., 8%, 10% or 12.5% poloxamer, e.g., poloxamer P407) and 1.2-2.75% (w/w) HA (e.g., 1.625% or 2.25% HA 730 KDa), incorporated with resiquimod, at a tumor resection site survived over a longer period of time, as compared to the control group receiving an immunomodulatory polymer combination without resiquimod. In addition, the group of tumor resection mice receiving such an immunomodulatory polymer combination incorporated with resiquimod exhibited a higher survival rate than the control group receiving an immunomodulatory polymer combination without resiquimod.

As shown in FIG. 29, the group of tumor resection mice receiving an immunomodulatory polymer combination of 8-12.5% (w/w) or 6-11% (w/w) poloxamer (e.g., 10% poloxamer, e.g., P407) and 1-5% (w/w) HA (e.g., 4% HA 119 KDa), incorporated with resiquimod, at a tumor resection site survived over a longer period of time as compared to the control group receiving an immunomodulatory polymer combination without resiquimod. In addition, the group of tumor resection mice receiving such an immunomodulatory polymer combination incorporated with resiquimod exhibited a higher survival rate than the control group receiving an immunomodulatory polymer combination without resiquimod.

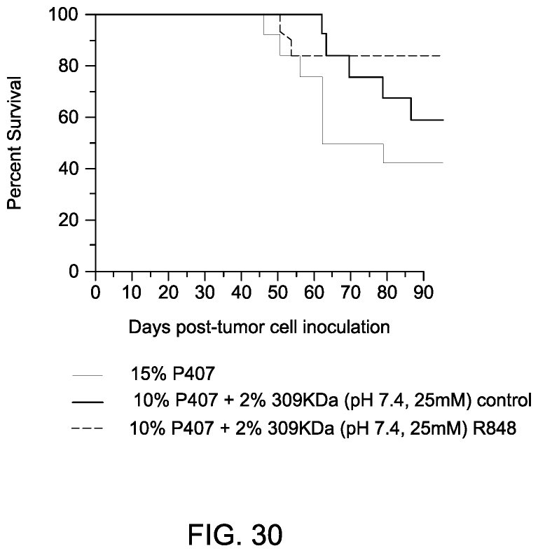

As shown in FIG. 30, the group of tumor resection mice receiving an immunomodulatory polymer combination of 8-12.5% (w/w) or 6-11% (w/w) poloxamer (e.g., 10% poloxamer e.g., P407) and 1-5% (w/w) HA (e.g., 2% HA 309 KDa), incorporated with or without resiquimod, at a tumor resection site survived over a longer period of time as compared to the control group receiving a 15% poloxamer only treatment without resiquimod.

The results demonstrated that polymer combination preparations described herein can be used in combination with resiquimod to treat subjects in need thereof, e.g., tumor resection subjects.

1. A method of preparing a composition comprising:
resiquimod; and
a polymer combination preparation comprising at least first and second polymer components, the first polymer component is or comprises a poloxamer and the second polymer component is not a poloxamer, the polymer combination preparation being characterized in that it transitions from a precursor state to a polymer network state in response to a gelation trigger,
wherein the polymer network state has a viscosity materially above that of the precursor state,
wherein the gelation trigger is or comprises temperature at or above critical gelation temperature (CGT) for the polymer combination preparation, ratio of polymer components at or above critical gelation weight ratio for the at least first and second polymer components, molecular weights of the at least first and/or second polymer components, or combinations thereof,
wherein the polymer network state comprises crosslinks not present in the precursor state;
wherein the crosslinks are or comprise intra-molecular crosslinks, inter-molecular crosslinks, or both; and
wherein the first polymer component is present in the polymer combination preparation at a concentration of 12.5% (w/w) or below,
said method comprising:
providing at least one solid form of resiquimod selected from the group consisting of Form I, Form II, Form III, Form IV, Form V, Form VI, and Form VII; and
combining the solid form of resiquimod with the first polymer component and the second polymer component,
to give the composition.

2. The method of claim 1, wherein the at least one solid form of resiquimod comprises Form I, wherein Form I is characterized by peaks in its XRPD pattern at about 8.72, about 12.24, about 16.29, about 17.56, about 19.51, about 21.31, and about 29.15 degrees 2-theta.

3. The method of claim 1, wherein the at least one solid form of resiquimod comprises Form II, wherein Form II is characterized by peaks in its XRPD pattern at about 7.75, about 9.65, about 11.23, about 14.38, about 19.90, about 20.80, and about 22.65 degrees 2-theta.

4. The method of claim 1, wherein the at least one solid form of resiquimod comprises Form III, wherein Form III is characterized by peaks in its XRPD pattern at about 8.69, about 9.18, about 9.48, about 11.97, about 14.41, about 18.53, and about 19.70 degrees 2-theta.

5. The method of claim 1, wherein the at least one solid form of resiquimod comprises Form IV, wherein Form IV is characterized by peaks in its XRPD pattern at about 6.01, about 12.00, about 12.15, about 16.14, about 19.24, about 20.21, about 21.19, about 22.12, and about 24.50 degrees 2-theta.

6. The method of claim 1, wherein the at least one solid form of resiquimod comprises Form V, wherein Form V is characterized by peaks in its XRPD pattern at about 8.13, about 10.20, about 10.44, about 16.29, and about 24.56 degrees 2-theta.

7. The method of claim 1, wherein the at least one solid form of resiquimod comprises Form VI, wherein Form VI is characterized by peaks in its XRPD pattern at about 9.40, about 13.02, about 18.13, about 18.93, about 20.38, about 23.16, and about 27.78 degrees 2-theta.

8. The method of claim 1, wherein the at least one solid form of resiquimod comprises Form VII, wherein Form VII is characterized by peaks in its XRPD pattern at about 6.25, about 9.92, about 10.96, about 16.51, about 18.99, about 23.75, and about 24.24 degrees 2-theta.

9. The method of any one of the preceding claims, wherein the method comprises:
mixing the solid form of resiquimod and the first polymer component in an appropriate buffer to provide a first mixture; and
combining the first mixture with the second polymer component,
to give the composition.

10. The method of any one of the preceding claims, wherein the polymer combination preparation does not comprise covalent crosslinks.

11. The method of any one of the preceding claims, wherein the CGT for the polymer combination preparation is 30-39° C. or 20-30° C.

12. The method of any one of the preceding claims, wherein the polymer combination preparation comprises a total polymer content of at least 5% (w/w), or at least 10% (w/w).

13. The method of any one of the preceding claims, wherein the polymer combination preparation comprises a total polymer content of at least 6% (w/w).

14. The method of any one of the preceding claims, wherein the critical gelation weight ratio of the first polymer component to the second polymer component is 1:1 to 14:1 or 1:1 to 10:1.

15. The method of any one of the preceding claims, wherein the critical gelation weight ratio of the first polymer component to the second polymer component is 1:1 to 22:1 or 1:1 to 18:1.

16. The method of any one of the preceding claims, wherein the polymer network state is a viscous solution or colloid.

17. The method of any one of the preceding claims, wherein the polymer network state is a hydrogel.

18. The method of any one of the preceding claims, wherein the second polymer component is or comprises a carbohydrate polymer.

19. The method of claim 18, wherein the carbohydrate polymer in the polymer combination preparation is present at concentration of below about 10% (w/w) or below about 5% (w/w).

20. The method of claim 19, wherein the carbohydrate polymer in the polymer combination preparation is present at concentration of below about 3% (w/w).

21. The method of any one of claims 18-20, wherein the carbohydrate polymer is or comprises hyaluronic acid.

22. The method of claim 21, wherein the hyaluronic acid has a molecular weight of about 50 kDa to about 2 MDa.

23. The method of claim 22, wherein the hyaluronic acid has a low molecular weight of about 100-500 kDa or about 100-400 kDa, or about 125-375 kDa, or about 100-200 kDa.

24. The method of any one of claims 18-20, wherein the carbohydrate polymer is or comprises chitosan or a modified chitosan.

25. The method of claim 24, wherein the modified chitosan is or comprises carboxymethyl chitosan.

26. The method of any one of the preceding claims, wherein the polymer network state is characterized by one or more material properties selected from:
a. storage modulus within a range of 100 Pa to about 10,000 Pa as measured at 37° C. and pH 5-8;
b. storage modulus that is at least 40% lower than that of a hydrogel formed from a solution having a poloxamer at a solution concentration of 18% (w/w); and
c. storage modulus, as measured at 37° C., that maintains substantially the same after its precursor state has been stored at 2-8° C. for a period of 1 month (or no more than 20% of the polymer combination preparation is degraded over a period of 1 month, when measured at 37° C.).

27. The method of any one of the preceding claims, wherein the polymer combination preparation has pH 4.5-8.5.

28. The method of any one of the preceding claims, wherein the polymer combination preparation has pH 7-8 (e.g., pH 7.4).

29. The method of any one of the preceding claims, wherein the polymer combination preparation has a higher buffering capacity than a 10 mM phosphate buffer.

30. The method of any one of the preceding claims, wherein the poloxamer is or comprises Poloxamer 407.

31. The method of any one of the preceding claims, wherein the composition is characterized in that a test animal group with spontaneous metastases having, at a tumor resection site, the composition comprising the polymer combination preparation in the polymer network state has a higher percent survival than a comparable test animal group having, at a tumor resection site, a composition without resiquimod, as assessed at 2 months after the administration.

32. The method of any one of the preceding claims, wherein the first polymer component is present in the polymer combination preparation at a concentration of 11% (w/w) or below.

33. The method of any one of the preceding claims, wherein the first polymer component is present in the polymer combination preparation at a concentration of 10.5% (w/w) or below.

34. The method of any one of the preceding claims, wherein the first polymer component is present in the polymer combination preparation at a concentration of 10% (w/w) or below.

35. The method of any one of the preceding claims, wherein the first polymer component is present in the polymer combination preparation at a concentration of at least 4% (w/w), at least 5% (w/w), or at least 6% (w/w).

36. The method of any one of the preceding claims, wherein the first polymer component is present in the polymer combination preparation at a concentration of at least 6% (w/w).

37. A composition prepared according to the method of any one of the preceding claims.

38. A composition comprising:
resiquimod;
8-12.5% (w/w) Poloxamer 407; and
1-4% (w/w) hyaluronic acid having a molecular weight of about 100 kDa to about 500 kDa.

39. The composition of claim 38, wherein the hyaluronic acid has a molecular weight of about 264 kDa to about 310 kDa.

40. The composition of claim 38 or claim 39, wherein the composition comprises 10% (w/w) Poloxamer 407.

41. The composition of any one of claims 38-40, wherein the composition comprises from 0.005 mg/mL to 1.00 mg/mL resiquimod.

42. The composition of any one of claims 38-41, wherein the composition comprises from 0.05 mg/mL to 0.20 mg/mL resiquimod.

43. The composition of any one of claims 38-42, wherein the composition has pH 7-8 (e.g., pH 7.4 or pH 8).

44. A method comprising administering the composition of any one of claims 37-43 to a subject in need thereof.

45. The method of claim 44, wherein the subject in need thereof is a subject suffering from cancer.

46. The method of claim 45, wherein the subject in need thereof is a subject suffering from or susceptible to recurrent or disseminated cancer.

47. The method of any one of claims 44-46, wherein the subject in need thereof is a tumor resection subject.

48. The method of claim 47, wherein the composition is administered at or within 2 cm of the tumor resection site.

49. The method of any one of claims 44-48, wherein the administration is by implantation.

50. The method of claim 49, wherein a composition comprising the polymer combination preparation in the polymer network state is administered by implantation.

51. The method of any one of claims 44-48, wherein the administration is by injection.

52. The method of claim 51, wherein a composition comprising the polymer combination preparation in the precursor state is administered by injection, wherein the precursor state transitions to the polymer network state upon the administration.

53. The method of any one of claims 44-52, wherein the administration is performed concurrently with or subsequent to laparoscopy.

54. The method of any one of claims 44-52, wherein the administration is performed concurrently with or subsequent to minimally invasive surgery.

55. The method of any one of claims 44-52, wherein the administration is performed concurrently with or subsequent to robotic surgery.

56. A kit comprising:
(i) at least one solid form of resiquimod selected from the group consisting of Form I, Form II, Form III, Form IV, Form V, Form VI, and Form VII;
(ii) a first polymer component; and
(iii) a second polymer component,
such that, when combined, a polymer combination preparation is provided, wherein the first polymer component is or comprises a poloxamer and the second polymer component is not a poloxamer, the polymer combination preparation being characterized in that it transitions from a precursor state to a polymer network state in response to a gelation trigger,
wherein the polymer network state has a viscosity materially above that of the precursor state,
wherein the gelation trigger is or comprises temperature at or above critical gelation temperature (CGT) for the polymer combination preparation, ratio of polymer components at or above critical gelation weight ratio for the at least first and second polymer components, molecular weights of the at least first and/or second polymer components, or combinations thereof,
wherein the polymer network state comprises crosslinks not present in the precursor state;
wherein the crosslinks are or comprise intra-molecular crosslinks, inter-molecular crosslinks, or both; and
wherein the first polymer component is present in the polymer combination preparation at a concentration of 12.5% (w/w) or below.

57. A composition comprising Resiquimod Form I and at least one solid form selected from the group consisting of Form II, Form III, Form IV, Form V, Form VI, and Form VII.

58. A method of preparing Resiquimod Form I, comprising:
(i) providing resiquimod;
(ii) dissolving resiquimod in a suitable solvent (e.g., dichloromethane); and
(iii) adding a suitable anti-solvent (e.g., heptane),
to give Resiquimod Form I.

59. A solid form of resiquimod, wherein the solid form is Form II and is characterized by peaks in its XRPD pattern at about 7.75, about 9.65, about 11.23, about 14.38, about 19.90, about 20.80, and about 22.65 degrees 2-theta.

60. A solid form of resiquimod, wherein the solid form is Form V and is characterized by peaks in its XRPD pattern at about 8.13, about 10.20, about 10.44, about 16.29, and about 24.56 degrees 2-theta.

61. A solid form of resiquimod, wherein the solid form is Form VII and is characterized by peaks in its XRPD pattern at about 6.25, about 9.92, about 10.96, about 16.51, about 18.99, about 23.75, and about 24.24 degrees 2-theta.
